(19) United States
(12) Patent Application Publication
(10) Pub. No.: US 2003/0190714 A1
(43) Pub. Date: Oct. 9, 2003

(54) Human ion channels

(76) Inventors: Steven L. Roberds, Mattawan, MI (US); Christopher W. Benjamin, Kalamazoo, MI (US); Alla M. Karnovsky, Kalamazoo, MI (US); Cara L. Ruble, Paw Paw, MI (US); Lisa I. Linske-O'Connell, Rockville, MD (US); Jun Wang, Gaithersburg, MD (US); Derong Liu, Germantown, MD (US)

Correspondence Address: COZEN O ' CONNOR, P.C., 1900 MARKET STREET, PHILADELPHIA, PA, 19103-3508, US

(21) Appl. No.: 09/802,668

(22) Filed: Mar. 9, 2001

Related U.S. Application Data
(60) Provisional application No. 60/188,517, filed on Mar. 10, 2000.
(60) Provisional application No. 60/188,519, filed on Mar. 10, 2000.
(60) Provisional application No. 60/188,484, filed on Mar. 10, 2000.
(60) Provisional application No. 60/188,518, filed on Mar. 10, 2000.
(60) Provisional application No. 60/188,400, filed on Mar. 10, 2000.
(60) Provisional application No. 60/215,815, filed on Jul. 5, 2000.
(60) Provisional application No. 60/216,481, filed on Jul. 6, 2000.

(51) Int. Cl. C12P 21/02; C07K 14/435; C07H 21/04; C12N 9/00

(52) U.S. Cl. 435/183; 530/350; 536/23.2; 435/69.1; 435/325; 435/320.1

(57) ABSTRACT

The present invention provides novel ion channel polypeptides and polynucleotides which identify and encode them. In addition, the invention provides expression vectors, host cells and methods for their production. The invention also provides methods for the identification of ion channel agonists/antagonists, useful for the treatment of human diseases and conditions.

### CROSS-REFERENCE TO RELATED APPLICATIONS

The present application claims priority of: Application Serial No. 60/188,517, filed Mar. 10, 2000; Application Serial No. 60/188,519, filed Mar. 10, 2000; Application Serial No. 60/188,484, filed Mar. 10, 2000; Application Serial No. 60/188,518, filed Mar. 10, 2000; Application Serial No. 60/188,400, filed Mar. 10, 2000; Application Serial No. 60/215,815, filed Jul. 5, 2000; and Application Serial No. 60/216,481, filed Jul. 6, 2000; each of which is hereby incorporated by reference in its entirety.

### FIELD OF THE INVENTION

The present invention is directed, in part, to nucleic acid molecules encoding ion channels, the novel polypeptides of these human ion channels, and assays for screening compounds that bind to these polypeptides and/or modulate their activities.

### BACKGROUND OF THE INVENTION

Ion channels are “molecular gates” that regulate the flow of ions into and out of cells. Ion flow plays an important role in all brain cell communication necessary for learning and memory. Additionally, ion flow is important in many physiological processes including, but not limited to, heart rate and body movement. Aberrations in ion channels have been implicated in, amongst other disorders, epilepsy, schizophrenia, Alzheimer's disease, migraine, arrhythmia, diabetes, and stroke damage. Ions flow down their electrochemical gradient through the ion channels (passive transport). The core of the channel is hydrophilic, and contains a part of the protein, the selectivity filter, which recognizes only certain ions and allows them to pass through. Channels are named by the ion(s) they allow to pass. Examples of ion channels include, but are not limited to, calcium channels, potassium channels, sodium channels, chloride channels, etc. An additional component of the channel is the gate. Only when the gate is open can the ions recognized by the selectivity filter pass through the channel. Gates open in response to a variety of stimuli, including, but not limited to, changes in membrane potential or the presence of certain chemicals outside or inside the cell. Channel names often also include an indication of what controls the gate: e.g., “voltage-gated calcium channel.” Presently, more than 50 different types of ion channels have been identified.

Communication between neurons is achieved by the release of neurotransmitters into the synapse. These neurotransmitters then activate receptors on the post-synaptic neuron. Many such receptors contain pores to rapidly conduct ions, such as sodium, calcium, potassium, and chloride, into the neuron. These pores, or channels, are made of protein subunits that are members of the family of proteins generally referred to as neurotransmitter-gated ion channel proteins. Included in this family are the serotonin 5-HT3 receptor, the gamma-aminobutyric-acid (GABA) receptor subunits, including gamma-1, rho-3, and beta-like, and the acetylcholine receptor protein subunits, including alpha-9 chain, epsilon chain, and beta-2 chain.

The neurotransmitter-gated ion channel superfamily includes 5-HT3, GABA_{A}, glutamate, glycine, and nicotinic acetylcholine receptor families. Within this superfamily, functional receptors are formed by homo- or heteropentamers of subunits having four transmembrane domains and an extracellular ligand-binding domain. The transmembrane domains of these receptors contribute to the formation of an ion pore.

Serotonin, also known as 5-hydroxytryptamine or 5-HT, is a biogenic amine that functions as a neurotransmitter, a mitogen and a hormone (Conley, E. C. (1995) The Ion Channels FactsBook Vol. I. Extracellular Ligand-Gated Channels, Academic Press, London and San Diego. pp. 426). Serotonin activates a large number of receptors, most of which are coupled to activation of G-proteins. However, 5-HT3 receptors are structurally distinct and belong to the neurotransmitter-gated ion channel superfamily. 5-HT3 receptors are expressed both pre- and post-synaptically on central and peripheral neurons. Post-synaptic 5-HT3 receptors achieve their effects by inducing excitatory potentials in the post-synaptic neuron, whereas pre-synaptic 5-HT3 receptors modulate the release of other neurotransmitters from the pre-synaptic neuron (Conley, 1995). 5-HT3 receptors have important roles in pain reception, cognition, cranial motor neuron activity, sensory processing and modulation of affect (Conley, 1995). Thus, ligands or drugs that modulate 5-HT3 receptors may be useful in treating pain, neuropathies, migraine, cognitive disorders, learning and memory deficits, Alzheimer's disease, Parkinson's disease, amyotrophic lateral sclerosis, emesis, cranial neuropathies, sensory deficits, anxiety, depression, schizophrenia, and other affective disorders.

Nicotinic acetylcholine receptors (AChR) are distinguished from other acetylcholine receptors by their affinity for nicotine and their structure-homo- or hetero-pentamers like all members of the neurotransmitter-gated ion channel superfamily. Nicotinic AChRs are found at the neuromuscular junction on skeletal muscle and on peripheral and central neurons. These receptors form nonselective cation channels and therefore induce excitatory currents when activated. Nicotinic AChRs are receptors for anesthetics, sedatives, and hallucinogens (Conley, 1995), and certain ligands have shown improvements in learning and memory in animals (Levin et al., Behavioral Pharmacology, 1999, 10:675-780). Thus, ligands or drugs that modulate nicotinic AChRs could be useful for anesthesia, sedation, improving learning and memory, improving cognition, schizophrenia, anxiety, depression, attention deficit hyperactivity disorder, and addiction or smoking cessation. Expression of AChR subunits is regulated during development enabling the design of ligands or drugs specifically targeted for particular developmental stages or diseases.

The neurotransmitter γ-aminobutyric acid (GABA) activates a family of neurotransmitter-gated ion channels (GABA_{A}) and a family of G protein-coupled receptors (GABA_{B}) (Conley, 1995). GABA_{A} receptors form chloride channels that induce inhibitory or hyperpolarizing currents when stimulated by GABA or GABA_{A} receptor agonists (Conley, 1995). GABA_{A} receptors are modulated by benzodiazepines, barbiturates, picrotoxin, and bicucuilline (Conley, 1995). Thus, ligands or drugs that modulate GABA_{A} receptors could be useful in sedation, anxiety, epilepsy, seizures, alcohol addiction or withdrawal, panic disorders, pre-menstrual syndrome, migraine, and other diseases characterized by hyper-excitability of central or peripheral neurons. The pharmacology of GABA_{A} receptors is affected by changing the subunit composition of the receptor. GABA receptor rho subunits are relatively specifically expressed in the retina (Cutting et al., 1991, Proc. Natl. Acad. Sci. USA, 88:2673-7), and the pharmacology of rho receptor homomultimers resembles that of so-called GABA_{C} receptors (Shimada et al., 1992, Mol. Pharmacol. 41:683-7). Therefore, GABA receptors consisting of rho subunits may be useful targets for discovering ligands or drugs to treat visual defects, macular degeneration, glaucoma, and other retinal disorders.

Compounds that modify the activity of these channels may also be useful for the control of neuromotor diseases including epilepsy and neurodegenerative diseases including Parkinson's and Alzheimer's. Also compounds that modulate the activity of these channels may treat diseases including but not limited to cardiovascular arrhythmias, stroke, and endocrine and muscular disorders.

Therefore, ion channels may be useful targets for discovering ligands or drugs to treat many diverse disorders and defects, including schizophrenia, depression, anxiety, attention deficit hyperactivity disorder, migraine, stroke, ischemia, and neurodegenerative disease such as Alzheimer's disease, Parkinson's disease, glaucoma and macular degeneration. In addition compounds which modulate ion channels can be used for the treatment of cardiovascular diseases including ischemia, congestive heart failure, arrhythmia, high blood pressure and restenosis.

### SUMMARY OF THE INVENTION

The present invention relates to an isolated nucleic acid molecule that comprises a nucleotide sequence that encodes a polypeptide comprising an amino acid sequence homologous to a sequence selected from the group consisting of SEQ ID NO:52 to SEQ ID NO:102, and SEQ ID NOS: 105, 106, 109, and 110, or a fragment thereof. The nucleic acid molecule encodes at least a portion of ion-x (where x is 31, 32, 33, 34, 35, 36, 37, 38, 39, 40, 41, 52, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, and 111). In some embodiments, the nucleic acid molecule comprises a sequence that encodes a polypeptide comprising a sequence selected from the group consisting of SEQ ID NO:52 to SEQ ID NO:102,, and SEQ ID NOS: 105, 106, 109, and 110, or a fragment thereof. In some embodiments, the nucleic acid molecule comprises a sequence homologous to a sequence selected from the group consisting of SEQ ID NO:1 to SEQ ID NO:5 1, and SEQ ID NOS:103, 104, 107, and 108, or a fragment thereof. In some embodiments, the nucleic acid molecule comprises a sequence selected from the group consisting of SEQ ID NO:1 to SEQ ID NO:51, and SEQ ID NOS:103, 104, 107, and 108, and fragments thereof.

According to some embodiments, the present invention provides vectors which comprise the nucleic acid molecule of the invention. In some embodiments, the vector is an expression vector.

According to some embodiments, the present invention provides host cells which comprise the vectors of the invention. In some embodiments, the host cells comprise expression vectors.

The present invention provides an isolated nucleic acid molecule comprising a nucleotide sequence complementary to at least a portion of a sequence selected from the group consisting of SEQ ID NO:1 to SEQ ID NO:51, and SEQ ID NOS:103, 104, 107, and 108, said portion comprising at least 10 nucleotides.

The present invention provides a method of producing a polypeptide comprising a sequence selected from the group consisting of SEQ ID NO:52 to SEQ ID NO:102, and SEQ ID NOS: 105, 106, 109, and 110, or a homolog or fragment thereof. The method comprising the steps of introducing a recombinant expression vector that includes a nucleotide sequence that encodes the polypeptide into a compatible host cell, growing the host cell under conditions for expression of the polypeptide and recovering the polypeptide.

The present invention provides an isolated antibody which binds to an epitope on a polypeptide comprising a sequence selected from the group consisting of SEQ ID NO:52 to SEQ ID NO:102, and SEQ ID NOS: 105, 106, 109, and 110, or a homolog or fragment thereof.

The present invention provides an method of inducing an immune response in a mammal against a polypeptide comprising a sequence selected from the group consisting of SEQ ID NO:52 to SEQ ID NO:102, and SEQ ID NOS: 105, 106, 109, and 110, or a homolog or fragment thereof. The method comprises administering to a mammal an amount of the polypeptide sufficient to induce said immune response.

The present invention provides a method for identifying a compound which binds ion-x. The method comprises the steps of: contacting ion-x with a compound and determining whether the compound binds ion-x. Compounds identified as binding ion-x may be further tested in other assays including, but not limited to, in vivo models, in order to confirm or quantitate their activity.

The present invention provides a method for identifying a compound which binds a nucleic acid molecule encoding ion-x. The method comprises the steps of contacting said nucleic acid molecule encoding ion-x with a compound and determining whether said compound binds said nucleic acid molecule.

The present invention provides a method for identifying a compound that modulates the activity of ion-x. The method comprises the steps of contacting ion-x with a compound and determining whether ion-x activity has been modulated. Compounds identified as modulating ion-x activity may be further tested in other assays including, but not limited to, in vivo models, in order to confirm or quantitate their activity.

The present invention provides a method of identifying an animal homolog of ion-x. The method comprises the steps screening a nucleic acid database of the animal with a sequence selected from the group consisting of SEQ ID NO:1 to SEQ ID NO:51, and SEQ ID NOS:103, 104, 107, and 108, or a portion thereof and determining whether a portion of said library or database is homologous to said sequence selected from the group consisting of SEQ ID NO:1 to SEQ ID NO:51, and SEQ ID NOS:103, 104, 107, and 108, or portion thereof.

The present invention provides a method of identifying an animal homolog of ion-x. The methods comprises the steps screening a nucleic acid library of the animal with a nucleic acid molecule having a sequence selected from the group consisting of SEQ ID NO:1 to SEQ ID NO:51, and SEQ ID NOS:103, 104, 107, and 108, or a portion thereof; and determining whether a portion of said library or database is homologous to said sequence selected from the group consisting of SEQ ID NO:1 to SEQ ID NO:51, and SEQ ID NOS:103, 104, 107, and 108, or a portion thereof.

Another aspect of the present invention relates to methods of screening a human subject to diagnose a disorder affecting the brain or genetic predisposition therefor. The methods comprise the steps of assaying nucleic acid of a human subject to determine a presence or an absence of a mutation altering an amino acid sequence, expression, or biological activity of at least one ion channel that is expressed in the brain. The ion channels comprise an amino acid sequence selected from the group consisting of: SEQ ID NO:52 to SEQ ID NO:102, and SEQ ID NOS: 105, 106, 109, and 110, and allelic variants thereof. A diagnosis of the disorder or predisposition is made from the presence or absence of the mutation. The presence of a mutation altering the amino acid sequence, expression, or biological activity of the ion channel in the nucleic acid correlates with an increased risk of developing the disorder.

The present invention further relates to methods of screening for an ion-x mental disorder genotype in a human patient. The methods comprise the steps of providing a biological sample comprising nucleic acid from the patient, in which the nucleic acid includes sequences corresponding to alleles of ion-x. The presence of one or more mutations in the ion-x allele is detected indicative of a mental disorder genotype. In some embodiments, the mental disorder includes, but is not limited to, schizophrenia, affective disorders, ADHD/ADD (i.e., Attention Deficit-Hyperactivity Disorder/Attention Deficit Disorder), and neural disorders such as Alzheimer's disease, Parkinson's disease, migraine, and senile dementia as well as depression, anxiety, bipolar disease, epilepsy, neuritis, neurasthenia, neuropathy, neuroses, and the like.

The present invention provides kits for screening a human subject to diagnose a mental disorder or a genetic predisposition therefor. The kits include an oligonucleotide useful as a probe for identifying polymorphisms in a human ion-x gene. The oligonucleotide comprises 6-50 nucleotides in a sequence that is identical or complementary to a sequence of a wild type human ion-x gene sequence or coding sequence, except for one sequence difference selected from the group consisting of a nucleotide addition, a nucleotide deletion, or nucleotide substitution. The kit also includes a media packaged with the oligonucleotide. The media contains information for identifying polymorphisms that correlate with a mental disorder or a genetic predisposition therefor, the polymorphisms being identifiable using the oligonucleotide as a probe.

The present invention further relates to methods of identifying ion channel allelic variants that correlates with mental disorders. The methods comprise the steps of providing biological samples that comprise nucleic acid from a human patient diagnosed with a mental disorder, or from the patient's genetic progenitors or progeny, and detecting in the nucleic acid the presence of one or more mutations in an ion channel that is expressed in the brain. The ion channel comprises an amino acid sequence selected from the group consisting of SEQ ID NO:52 to SEQ ID NO:102, and SEQ ID NOS: 105, 106, 109, and 110, and allelic variants thereof. The nucleic acid includes sequences corresponding to the gene or genes encoding ion-x. The one or more mutations detected indicate an allelic variant that correlates with a mental disorder.

The present invention further relates to purified polynucleotides comprising nucleotide sequences encoding alleles of ion-x from a human with a mental disorder. The polynucleotide hybridizes to the complement of SEQ ID NO:1 to SEQ ID NO:51 and SEQ ID NOS:103, 104, 107, and 108, under the following hybridization conditions: (a) hybridization for 16 hours at 42° C. in a hybridization solution comprising 50% formamide, 1% SDS, 1 M NaCl, 10% dextran sulfate and (b) washing 2 times for 30 minutes at 60° C. in a wash solution comprising 0.1×SSC and 1% SDS. The polynucleotide that encodes ion-x amino acid sequence of the human differs from SEQ ID NO052 to SEQ ID NO:102, and SEQ ID NOS: 105, 106, 109, and 110, by at least one residue.

The present invention also provides methods for identifying a modulator of biological activity of ion-x comprising the steps of contacting a cell that expresses ion-x in the presence and in the absence of a putative modulator compound and measuring ion-x biological activity in the cell. The decreased or increased ion-x biological activity in the presence versus absence of the putative modulator is indicative of a modulator of biological activity. Compounds identified as modulating ion-x activity may be further tested in other assays including, but not limited to, in vivo models, in order to confirm or quantitate their activity.

As used herein, the term “biological activity” of an ion channel refers to the native activity of the ion channel. Activities of ion channels include, but are not limited to, the ability to bind or be affected by certain compounds, and the ability to transport ions from one side of the membrane to the other side.

The present invention further provides methods to identify compounds useful for the treatment of mental disorders. The methods comprise the steps of contacting a composition comprising ion-x with a compound suspected of binding ion-x. The binding between ion-x and the compound suspected of binding ion-x is detected. Compounds identified as binding ion-x are candidate compounds useful for the treatment of mental disorders.

The present invention further provides methods for identifying a compound useful as a modulator of binding between ion-x and a binding partner of ion-x. The methods comprise the steps of contacting the binding partner and a composition comprising ion-x in the presence and in the absence of a putative modulator compound and detecting binding between the binding partner and ion-x. Decreased or increased binding between the binding partner and ion-x in the presence of the putative modulator, as compared to binding in the absence of the putative modulator is indicative a modulator compound useful for the treatment of mental disorders.

The present invention further provides chimeric receptors comprising at least a portion of a sequence selected from the group consisting of SEQ ID NO:1 to SEQ ID NO:51, and SEQ ID NOS:103, 104, 107, and 108, said portion comprising at least 10 nucleotides.

These and other aspects of the invention are described in greater detail below.

### DETAILED DESCRIPTION OF PREFERRED EMBODIMENTS

The present invention provides, inter alia, isolated and purified polynucleotides that encode human ion channels or a portion thereof, vectors containing these polynucleotides, host cells transformed with these vectors, processes of making ion channels and subunits, methods of using the above polynucleotides and vectors, isolated and purified ion channels and subunits, methods of screening compounds which modulate ion channel activity, and compounds that modulate ion channel activity.

Definitions

Various definitions are made throughout this document. Most words have the meaning that would be attributed to those words by one skilled in the art. Words specifically defined either below or elsewhere in this document have the meaning provided in the context of the present invention as a whole and as typically understood by those skilled in the art.

As used herein, the phrase “ion channel” refers to an entire channel that allows the movement of ions across a membrane, as well as to subunit polypeptide chains that comprise such a channel. As the ion channels of the present inventions are ligand-gated, the ion channels are also referred to as “receptors.” Those of skill in the art will recognize that ion channels are made of subunits. As used herein, the term “subunit” refers to any component portion of an ion channel, including but not limited to the beta subunit and other associated subunits.

“Synthesized” as used herein and understood in the art, refers to polynucleotides produced by purely chemical, as opposed to enzymatic, methods. “Wholly” synthesized DNA sequences are therefore produced entirely by chemical means, and “partially” synthesized DNAs embrace those wherein only portions of the resulting DNA were produced by chemical means.

By the term “region” is meant a physically contiguous portion of the primary structure of a biomolecule. In the case of proteins, a region is defined by a contiguous portion of the amino acid sequence of that protein.

The term “domain” is herein defined as referring to a structural part of a biomolecule that contributes to a known or suspected function of the biomolecule. Domains may be co-extensive with regions or portions thereof; domains may also incorporate a portion of a biomolecule that is distinct from a particular region, in addition to all or part of that region. Examples of ion channel domains include, but are not limited to, the extracellular (i.e., N-terminal), transmembrane and cytoplasmic (i.e., C-terminal) domains, which are co-extensive with like-named regions of ion channels; and each of the loop segments (both extracellular and intracellular loops) connecting adjacent transmembrane segments.

As used herein, the term “activity” refers to a variety of measurable indicia suggesting or revealing binding, either direct or indirect; affecting a response, i.e., having a measurable affect in response to some exposure or stimulus, including, for example, the affinity of a compound for directly binding a polypeptide or polynucleotide of the invention. Activity can also be determined by measurement of downstream enzyme activities, and downstream messengers such as K⁺ ions, Ca²⁺ ions, Na⁺ ions, Cl⁻ ions, cyclic AMP, and phospholipids after some stimulus or event. For example, activity can be determined by measuring ion flux. As used herein, the term “ion flux” includes ion current. Activity can also be measured by measuring changes in membrane potential using electrodes or voltage-sensitive dyes, or by measuring neuronal or cellular activity such as action potential duration or frequency, the threshold for stimulating action potentials, long-term potentiation, or long-term inhibition.

As used herein, the term “protein” is intended to include full length and partial fragments of proteins. The term “protein” may be used, herein, interchangeably with “polypeptide.” Thus, as used herein, the term “protein” includes polypeptide, peptide, oligopeptide, or amino acid sequence.

As used herein, the term “chimeric receptor” is intended to refer to a receptor comprising portions of more than one type of receptor. As a non-limiting example, a chimeric receptor may comprise the pore-forming transmembrane domain of an alpha9 nicotinic acetylcholine receptor and the extracellular domain of the alphalo nicotinic acetylcholine receptor. Chimeric receptors of the present invention are not limited to hybrids of related receptors; chimeric receptors may also include, for example, the pore-forming transmembrane domain of an alpha7 nicotinic acetylcholine receptor and the extracellular domain of the GABA receptor. Chimeric receptors may also include portions of known wild-type receptors and portions of artificial receptors.

As used herein, the term “antibody” is meant to refer to complete, intact antibodies, Fab fragments, and F(ab)₂ fragments thereof. Complete, intact antibodies include monoclonal antibodies such as murine monoclonal antibodies, polyclonal antibodies, chimeric antibodies, humanized antibodies, and recombinant antibodies identified using phage display.

As used herein, the term “binding” means the physical or chemical interaction between two proteins, compounds or molecules (including nucleic acids, such as DNA or RNA), or combinations thereof. Binding includes ionic, non-ionic, hydrogen bonds, Van der Waals, hydrophobic interactions, etc. The physical interaction, the binding, can be either direct or indirect, indirect being through or due to the effects of another protein, compound or molecule. Direct binding refers to interactions that do not take place through or due to the effect of another protein, compound or molecule, but instead are without other substantial chemical intermediates. Binding may be detected in many different manners. As a non-limiting example, the physical binding interaction between an ion channel of the invention and a compound can be detected using a labeled compound. Alternatively, functional evidence of binding can be detected using, for example, a cell transfected with and expressing an ion channel of the invention. Binding of the transfected cell to a ligand of the ion channel that was transfected into the cell provides functional evidence of binding. Other methods of detecting binding are well known to those of skill in the art.

As used herein, the term “compound” means any identifiable chemical or molecule, including, but not limited to a small molecule, peptide, protein, sugar, nucleotide, or nucleic acid. Such compound can be natural or synthetic.

As used herein, the term “complementary” refers to Watson-Crick base-pairing between nucleotide units of a nucleic acid molecule.

As used herein, the term “contacting” means bringing together, either directly or indirectly, a compound into physical proximity to a polypeptide or polynucleotide of the invention. The polypeptide or polynucleotide can be present in any number of buffers, salts, solutions, etc. Contacting includes, for example, placing the compound into a beaker, microtiter plate, cell culture flask, or a microarray, such as a gene chip, or the like, which contains either the ion channel polypeptide or fragment thereof, or nucleic acid molecule encoding an ion channel or fragment thereof.

As used herein, the phrase “homologous nucleotide sequence,” or “homologous amino acid sequence,” or variations thereof, refers to sequences characterized by a homology, at the nucleotide level or amino acid level, of at least about 60%, more preferably at least about 70%, more preferably at least about 80%, more preferably at least about 90%, and most preferably at least about 95% to the entirety of SEQ ID NO:1 to SEQ ID NO:51, and SEQ ID NOS:103, 104, 107, and 108, or to at least a portion of SEQ ID NO:1 to SEQ ID NO:51, and SEQ ID NOS:103, 104, 107, and 108, which portion encodes a functional domain of the encoded polypeptide, or to SEQ ID NO:52 to SEQ ID NO:102, and SEQ ID NOS: 105, 106, 109, and 110. Homologous nucleotide sequences include those sequences coding for isoforms of ion channel proteins. Such isoforms can be expressed in different tissues of the same organism as a result of, for example, alternative splicing of RNA. Alternatively, isoforms can be encoded by different genes. Homologous nucleotide sequences include nucleotide sequences encoding for an ion channel protein of a species other than human, including, but not limited to, mammals. Homologous nucleotide sequences also include, but are not limited to, naturally occurring allelic variations and mutations of the nucleotide sequences set forth herein. Although the present invention provides particular sequences, it is understood that the invention is intended to include within its scope other human allelic variants and non-human forms of the ion channels described herein.

Homologous amino acid sequences include those amino acid sequences which contain conservative amino acid substitutions in SEQ ID NO:52 to SEQ ID NO:102, and SEQ ID NOS: 105, 106, 109, and 110, as well as polypeptides having ion channel activity. A homologous amino acid sequence does not, however, include the sequence of known polypeptides having ion channel activity. Percent homology can be determined by, for example, the Gap program (Wisconsin Sequence Analysis Package, Version 8 for Unix, Genetics Computer Group, University Research Park, Madison Wis.), which uses the algorithm of Smith and Waterman (Adv. Appl. Math., 1981, 2, 482-489, which is incorporated herein by reference in its entirety) using the default settings. As used herein, the term “percent homology” and its variants are used interchangeably with “percent identity” and “percent similarity.”

As used herein, the term “isolated” nucleic acid molecule refers to a nucleic acid molecule (DNA or RNA) that has been removed from its native environment. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules.

As used herein, the terms “modulates” or “modifies” means an increase or decrease in the amount, quality, or effect of a particular activity or protein.

The term “preventing” refers to decreasing the probability that an organism contracts or develops an abnormal condition.

The term “treating” refers to having a therapeutic effect and at least partially alleviating or abrogating an abnormal condition in the organism.

The term “therapeutic effect” refers to the inhibition or activation factors causing or contributing to the abnormal condition. A therapeutic effect relieves to some extent one or more of the symptoms of the abnormal condition. In reference to the treatment of abnormal conditions, a therapeutic effect can refer to one or more of the following: (a) an increase in the proliferation, growth, and/or differentiation of cells; (b) inhibition (i.e., slowing or stopping) of cell death; (c) inhibition of degeneration; (d) relieving to some extent one or more of the symptoms associated with the abnormal condition; and (e) enhancing the function of the affected population of cells. Compounds demonstrating efficacy against abnormal conditions can be identified as described herein.

The term “abnormal condition” refers to a function in the cells or tissues of an organism that deviates from their normal functions in that organism. An abnormal condition can relate to cell proliferation, cell differentiation, cell signaling, or cell survival. An abnormal condition may also include obesity, diabetic complications such as retinal degeneration, and irregularities in glucose uptake and metabolism, and fatty acid uptake and metabolism.

Abnormal cell proliferative conditions include cancers such as fibrotic and mesangial disorders, abnormal angiogenesis and vasculogenesis, wound healing, psoriasis, diabetes mellitus, and inflammation.

Abnormal differentiation conditions include, but are not limited to, neurodegenerative disorders, slow wound healing rates, and slow tissue grafting healing rates. Abnormal cell signaling conditions include, but are not limited to, psychiatric disorders involving excess neurotransmitter activity.

Abnormal cell survival conditions may also relate to conditions in which programmed cell death (apoptosis) pathways are activated or abrogated. A number of protein kinases are associated with the apoptosis pathways. Aberrations in the function of any one of the protein kinases could lead to cell immortality or premature cell death.

The term “administering” relates to a method of incorporating a compound into cells or tissues of an organism. The abnormal condition can be prevented or treated when the cells or tissues of the organism exist within the organism or outside of the organism. Cells existing outside the organism can be maintained or grown in cell culture dishes. For cells harbored within the organism, many techniques exist in the art to administer compounds, including (but not limited to) oral, parenteral, dermal, injection, and aerosol applications. For cells outside of the organism, multiple techniques exist in the art to administer the compounds, including (but not limited to) cell microinjection techniques, transformation techniques and carrier techniques.

The abnormal condition can also be prevented or treated by administering a compound to a group of cells having an aberration in ion channel in an organism. The effect of administering a compound on organism function can then be monitored. The organism is preferably a mouse, rat, rabbit, guinea pig or goat, more preferably a monkey or ape, and most preferably a human.

By “amplification” it is meant increased numbers of DNA or RNA in a cell compared with normal cells. “Amplification” as it refers to RNA can be the detectable presence of RNA in cells, since in some normal cells there is no basal expression of RNA. In other normal cells, a basal level of expression exists, therefore, in these cases amplification is the detection of at least 1 to 2-fold, and preferably more, compared to the basal level.

As used herein, the term “oligonucleotide” refers to a series of linked nucleotide residues which has a sufficient number of bases to be used in a polymerase chain reaction (PCR). This short sequence is based on (or designed from) a genomic or cDNA sequence and is used to amplify, confirm, or reveal the presence of an identical, similar or complementary DNA or RNA in a particular cell or tissue. Oligonucleotides comprise portions of a nucleic acid sequence having at least about 10 nucleotides and as many as about 50 nucleotides, preferably about 15 to 30 nucleotides. They are chemically synthesized and may be used as probes.

As used herein, the term “probe” refers to nucleic acid sequences of variable length, preferably between at least about 10 and as many as about 6,000 nucleotides, depending on use. They are used in the detection of identical, similar, or complementary nucleic acid sequences. Longer length probes are usually obtained from a natural or recombinant source, are highly specific and much slower to hybridize than oligomers. They may be single- or double-stranded and are carefully designed to have specificity in PCR, hybridization membrane-based, or ELISA-like technologies.

As used herein, the phrase “stringent hybridization conditions” or “stringent conditions” refers to conditions under which a probe, primer, or oligonucleotide will hybridize to its target sequence, but to a minimal number of other sequences. Stringent conditions are sequence-dependent and will be different in different circumstances. Longer sequences will hybridize with specificity to their proper complements at higher temperatures. Generally, stringent conditions are selected to be about 5° C. lower than the thermal melting point (Tₘ) for the specific sequence at a defined ionic strength and pH. The Tₘ is the temperature (under defined ionic strength, pH and nucleic acid concentration) at which 50% of the probes complementary to the target sequence hybridize to the target sequence at equilibrium. Since the target sequences are generally present in excess, at Tₘ, 50% of the probes are hybridized to their complements at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 to 50 nucleotides) and at least about 60° C. for longer probes, primers or oligonucleotides. Stringent conditions may also be achieved with the addition of destabilizing agents, such as formamide.

The amino acid sequences are presented in the amino (N) to carboxy (C) direction, from left to right. The N-terminal α-amino group and the C-terminal β-carboxy groups are not depicted in the sequence. The nucleotide sequences are presented by single strands only, in the 5′ to 3′ direction, from left to right. Nucleotides and amino acids are represented in the manner recommended by the IUPAC-IUB Biochemical Nomenclature Commission, or amino acids are represented by their three letters code designations.

Polynucleotides

The present invention provides purified and isolated polynucleotides (e.g., DNA sequences and RNA transcripts, both sense and complementary antisense strands, both single- and double-stranded, including splice variants thereof) that encode unknown ion channels. These genes are described herein and designated herein collectively as ion-x (where x is 31, 32, 33, 34, 35, 36, 37, 38, 39, 40, 41, 52, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, and 111). That is, these genes and gene products are described herein and designated herein as ion-31, ion-32, ion-33, ion-34, ion-35, ion-36, ion-37, ion-38, ion-39, ion-40, ion-41, ion-52, ion-56, ion-57, ion-58, ion-59, ion-60, ion-61, ion-62, ion-63, ion-64, ion-65, ion-66, ion-67, ion-68, ion-69, ion-70, ion-71, ion-72, ion-73, ion-74, ion-75, ion-76, ion-77, ion-78, ion-79, ion-80, ion-81, ion-82, ion-83, ion-85, ion-86, ion-87, ion-88, ion-89, ion-89, ion-90, ion-91, ion-92, ion-93, ion-94, ion-95, and ion-111. Table 1 below identifies the novel gene sequence ion-x designation, the SEQ ID NO: of the gene sequence, and the SEQ ID NO: of the polypeptide encoded thereby.

TABLE 1

| | Nucleotide | Amino acid | |
|---|---|---|---|
| | Sequence | Sequence | |
| | (SEQ ID | (SEQ ID | Originally |
| ion-x | NO:) | NO:) | filed in: |
| 31 | 1 | 52 | A |
| 32 | 2 | 53 | A |
| 33 | 3 | 54 | A |
| 34 | 4 | 55 | A |
| 35 | 5 | 56 | A |
| 36 | 6 | 57 | A |
| 37 | 7 | 58 | A |
| 38 | 8 | 59 | A |
| 39 | 9 | 60 | A |
| 40 | 10 | 61 | A |
| 41 | 11 | 62 | A |
| 52 | 107 | 109 | F |
| 56 | 12 | 63 | B |
| 57 | 13 | 64 | B |
| 58 | 14 | 65 | B |
| 59 | 15 | 66 | B |
| 60 | 16 | 67 | B |
| 61 | 17 | 68 | B |
| 62 | 18 | 69 | B |
| 63 | 19 | 70 | B |
| 64 | 20 | 71 | B |
| 65 | 21 | 72 | B |
| 66 | 22 | 73 | C |
| 67 | 23 | 74 | C |
| 68 | 24 | 75 | C |
| 69 | 25 | 76 | C |
| 70 | 26 | 77 | C |
| 71 | 27 | 78 | C |
| 72 | 28 | 79 | C |
| 73 | 29 | 80 | C |
| 74 | 30 | 81 | C |
| 75 | 31 | 82 | C |
| 76 | 32 | 83 | D |
| 77 | 33 | 84 | D |
| 78 | 34 | 85 | D |
| 79 | 35 | 86 | D |
| 80 | 36 | 87 | D |
| 81 | 37 | 88 | D |
| 82 | 38 | 89 | D |
| 83 | 39 | 90 | D |
| 84 | 40 | 91 | D |
| 85 | 41 | 92 | D |
| 86 | 42 | 93 | E |
| 87 | 43 | 94 | E |
| 88 | 44 | 95 | E |
| 89 | 45 | 96 | E |
| 90 | 46 | 97 | E |
| 91 | 47 | 98 | E |
| 92 | 48 | 99 | E |
| 93 | 49 | 100 | E |
| 94 | 50 | 101 | E |
| 95 | 51 | 102 | E |
| 111 | 108 | 110 | G |

Legend
A = Ser. No. 60/188,517
B = Ser. No. 60/188,519
C = Ser. No. 60/188,484
D = Ser. No. 60/188,518
E = Ser. No. 60/188,400
F = Ser. No. 60/215,815
G = Ser. No. 60/216,481

When a specific ion-x is identified (for example ion-65), it is understood that only cific ion channel is being referred to.

The invention provides purified and isolated polynucleotides (e.g., cDNA, genornic DNA, synthetic DNA, RNA, or combinations thereof, whether single- or double-stranded) that comprise a nucleotide sequence encoding the amino acid sequence of the polypeptides of the invention. Such polynucleotides are useful for recombinantly expressing the receptor and also for detecting expression of the receptor in cells (e.g., using Northern hybridization and in situ hybridization assays). Such polynucleotides also are useful in the design of antisense and other molecules for the suppression of the expression of ion-x in a cultured cell, a tissue, or an animal; for therapeutic purposes; or to provide a model for disease or conditions characterized by aberrant ion-x expression. Specifically excluded from the definition of polynucleotides of the invention are entire isolated, non-recombinant native chromosomes of host cells. A preferred polynucleotide has a sequence selected from the group consisting of SEQ ID NO:1 to SEQ ID NO:51, and SEQ ID NOS:103, 104, 107, and 108, which correspond to naturally occurring ion-x sequences. It will be appreciated that numerous other polynucleotide sequences exist that also encode ion-x having sequence selected from the group consisting of SEQ ID NO:52 to SEQ ID NO:102, and SEQ ID NOS: 105, 106, 109, and 110, due to the well-known degeneracy of the universal genetic code.

The invention also provides a purified and isolated polynucleotide comprising a nucleotide sequence that encodes a mammalian polypeptide, wherein the polynucleotide hybridizes to a polynucleotide having a sequence selected from the group consisting of SEQ ID NO:1 to SEQ ID NO:51, and SEQ ID NOS:103, 104, 107, and 108, or the non-coding strand complementary thereto, under the following hybridization conditions:

(a) hybridization for 16 hours at 42° C. in a hybridization solution comprising 50% formamide, 1% SDS, 1 M NaCl, 10% dextran sulfate; and

(b) washing 2 times for 30 minutes each at 60° C. in a wash solution comprising 0.1% SSC, 1% SDS. Polynucleotides that encode a human allelic variant are highly preferred.

The present invention relates to molecules which comprise the gene sequences that encode the ion channels; constructs and recombinant host cells incorporating the gene sequences; the novel ion-x polypeptides encoded by the gene sequences; antibodies to the polypeptides and homologs; kits employing the polynucleotides and polypeptides, and methods of making and using all of the foregoing. In addition, the present invention relates to homologs of the gene sequences and of the polypeptides and methods of making and using the same.

Genomic DNA of the invention comprises the protein-coding region for a polypeptide of the invention and is also intended to include allelic variants thereof. It is widely understood that, for many genes, genomic DNA is transcribed into RNA transcripts that undergo one or more splicing events wherein intron (i.e., non-coding regions) of the transcripts are removed, or “spliced out.” RNA transcripts that can be spliced by alternative mechanisms, and therefore be subject to removal of different RNA sequences but still encode an ion-x polypeptide, are referred to in the art as splice variants which are embraced by the invention. Splice variants comprehended by the invention therefore are encoded by the same original genomic DNA sequences but arise from distinct mRNA transcripts. Allelic variants are modified forms of a wild-type gene sequence, the modification resulting from recombination during chromosomal segregation or exposure to conditions which give rise to genetic mutation. Allelic variants, like wild type genes, are naturally occurring sequences (as opposed to non-naturally occurring variants that arise from in vitro manipulation).

The invention also comprehends cDNA that is obtained through reverse transcription of an RNA polynucleotide encoding ion-x (conventionally followed by second strand synthesis of a complementary strand to provide a double-stranded DNA).

Preferred DNA sequences encoding human ion-x polypeptides are set out in sequences selected from the group consisting of SEQ ID NO:1 to SEQ ID NO:5 1, and SEQ ID NOS:103, 104, 107, and 108. A preferred DNA of the invention comprises a double stranded molecule along with the complementary molecule (the “non-coding strand” or “complement”) having a sequence unambiguously deducible from the coding strand according to Watson-Crick base-pairing rules for DNA. Also preferred are other polynucleotides encoding the ion-x polypeptide of sequences selected from the group consisting of SEQ ID NO:52 to SEQ ID NO:102, and SEQ ID NOS: 105, 106, 109, and 110, which differ in sequence from the polynucleotides of sequences selected from the group consisting of SEQ ID NO:1 to SEQ ID NO:51, and SEQ ID NOS:103, 104, 107, and 108, by virtue of the well-known degeneracy of the universal nuclear genetic code.

In a preferred embodiment, the isolated nucleic acid molecule comprises a nucleotide sequence which encodes a polypeptide comprising a sequence of SEQ ID NO:105 or 106. The polypeptide comprising a sequence of SEQ ID NO:105 or 106 comprises at least one or more amino acid residues from one or more of the following sequences: ion31 (SEQ ID NO:52), ion52 (SEQ ID NO: 109), and ion111 (SEQ ID NO:110).

In a preferred embodiment, the isolated nucleic acid comprises a nucleotide sequence of SEQ ID NO: 103 or 104, that encode a polypeptide having a sequence of SEQ ID NO:105 or 106. The isolated nucleic acid encoding a polypeptide having a sequence of SEQ ID NO:105 or 106 comprises at least one or more nucleotides from one or more of the following sequences: ion31 (SEQ ID NO:1), ion52 (SEQ ID NO:107), and ion111 (SEQ ID NO108).

The invention further embraces other species, preferably mammalian, homologs of the human ion-x DNA. Species homologs, sometimes referred to as “orthologs,” in general, share at least 35%, at least 40%, at least 45%, at least 50%, at least 60%, at least 65%, at least 70%, at least 75%, at least 80%, at least 85%, at least 90%, at least 95%, at least 98%, or at least 99% homology with human DNA of the invention. Generally, percent sequence “homology” with respect to polynucleotides of the invention may be calculated as the percentage of nucleotide bases in the candidate sequence that are identical to nucleotides in the ion-x sequence selected from the group consisting of SEQ ID NO:1 to SEQ ID NO:51, and SEQ ID NOS:103, 104, 107, and 108, after aligning the sequences and introducing gaps, if necessary, to achieve the maximum percent sequence identity.

Polynucleotides of the invention permit identification and isolation of polynucleotides encoding related ion-x polypeptides, such as human allelic variants and species homologs, by well-known techniques including Southern and/or Northern hybridization, and polymerase chain reaction (PCR). Examples of related polynucleotides include human and non-human genomic sequences, including allelic variants, as well as polynucleotides encoding polypeptides homologous to ion-x and structurally related polypeptides sharing one or more biological, immunological, and/or physical properties of ion-x. Non-human species genes encoding proteins homologous to ion-x can also be identified by Southern and/or PCR analysis and are useful in animal models for ion-x disorders. Knowledge of the sequence of a human ion-x DNA also makes possible through use of Southern hybridization or polymerase chain reaction (PCR) the identification of genomic DNA sequences encoding ion-x expression control regulatory sequences such as promoters, operators, enhancers, repressors, and the like. Polynucleotides of the invention are also useful in hybridization assays to detect the capacity of cells to express ion-x. Polynucleotides of the invention may also provide a basis for diagnostic methods useful for identifying a genetic alteration(s) in an ion-x locus that underlies a disease state or states, which information is useful both for diagnosis and for selection of therapeutic strategies.

According to the present invention, the ion-x nucleotide sequences disclosed herein may be used to identify homologs of the ion-x, in other animals, including but not limited to humans and other mammals, and invertebrates. Any of the nucleotide sequences disclosed herein, or any portion thereof, can be used, for example, as probes to screen databases or nucleic acid libraries, such as, for example, genomic or cDNA libraries, to identify homologs, using screening procedures well known to those skilled in the art.

Fragment polynucleotides are particularly useful as probes for detection of full-length or fragments of ion-x polynucleotides. One or more polynucleotides can be included in kits that are used to detect the presence of a polynucleotide encoding ion-x, or used to detect variations in a polynucleotide sequence encoding ion-x.

The invention also embraces DNAs encoding ion-x polypeptides that hybridize under moderately stringent or high stringency conditions to the non-coding strand, or complement, of the polynucleotides set forth in a sequence selected from the group consisting of SEQ ID NO:1 to SEQ ID NO:51, and SEQ ID NOS:103, 104, 107, and 108.

Exemplary highly stringent hybridization conditions are as follows: hybridization at 42° C. in a hybridization solution comprising 50% formamide, 1% SDS, 1 M NaCl, 10% Dextran sulfate, and washing twice for 30 minutes at 60° C. in a wash solution comprising 0.1×SSC and 1% SDS. It is understood in the art that conditions of equivalent stringency can be achieved through variation of temperature and buffer, or salt concentration as described Ausubel et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook et al., (Eds.), Molecular Cloning: A Laboratory Manual, Cold Spring Harbor Laboratory Press: Cold Spring Harbor, N.Y. (1989), pp. 9.47 to 9.51.

With the knowledge of the nucleotide sequence information disclosed in the present invention, one skilled in the art can identify and obtain nucleotide sequences which encode ion-x from different sources (i.e., different tissues or different organisms) through a variety of means well known to the skilled artisan and as disclosed by, for example, Sambrook et al., “Molecular cloning: a laboratory manual”, Second Edition, Cold Spring Harbor Press, Cold Spring Harbor, N.Y. (1989), which is incorporated herein by reference in its entirety.

For example, DNA that encodes ion-x may be obtained by screening MRNA, cDNA, or genomic DNA with oligonucleotide probes generated from the ion-x gene sequence information provided herein. Probes may be labeled with a detectable group, such as a fluorescent group, a radioactive atom or a chemiluminescent group in accordance with procedures known to the skilled artisan and used in conventional hybridization assays, as described by, for example, Sambrook et al.

A nucleic acid molecule comprising any of the ion-x nucleotide sequences described above can alternatively be synthesized by use of the polymerase chain reaction (PCR) procedure, with the PCR oligonucleotide primers produced from the nucleotide sequences provided herein. See U.S. Pat. No. 4,683,195 to Mullis et al. and U.S. Pat. No. 4,683,202 to Mullis. The PCR reaction provides a method for selectively increasing the concentration of a particular nucleic acid sequence even when that sequence has not been previously purified and is present only in a single copy in a particular sample. The method can be used to amplify either single- or double-stranded DNA. The essence of the method involves the use of two oligonucleotide probes to serve as primers for the template-dependent, polymerase mediated replication of a desired nucleic acid molecule.

A wide variety of alternative cloning and in vitro amplification methodologies are well known to those skilled in the art. Examples of these techniques are found in, for example, Berger et al., Guide to Molecular Cloning Techniques, Methods in Enzymology 152, Academic Press, Inc., San Diego, Calif. (Berger), which is incorporated herein by reference in its entirety.

Automated sequencing methods can be used to obtain or verify the nucleotide sequence of ion-x. The ion-x nucleotide sequences of the present invention are believed to be 100% accurate. However, as is known in the art, nucleotide sequence obtained by automated methods may contain some errors. Nucleotide sequences determined by automation are typically at least about 90%, more typically at least about 95% to at least about 99.9% identical to the actual nucleotide sequence of a given nucleic acid molecule. The actual sequence may be more precisely determined using manual sequencing methods, which are well known in the art. An error in a sequence which results in an insertion or deletion of one or more nucleotides may result in a frame shift in translation such that the predicted amino acid sequence will differ from that which would be predicted from the actual nucleotide sequence of the nucleic acid molecule, starting at the point of the mutation.

The nucleic acid molecules of the present invention, and fragments derived therefrom, are useful for screening for restriction fragment length polymorphism (RFLP) associated with certain disorders, as well as for genetic mapping.

The polynucleotide sequence information provided by the invention makes possible large-scale expression of the encoded polypeptide by techniques well known and routinely practiced in the art.

Vectors

Another aspect of the present invention is directed to vectors, or recombinant expression vectors, comprising any of the nucleic acid molecules described above. Vectors are used herein either to amplify DNA or RNA encoding ion-x and/or to express DNA which encodes ion-x. Preferred vectors include, but are not limited to, plasmids, phages, cosmids, episomes, viral particles or viruses, and integratable DNA fragments (i.e., fragments integratable into the host genome by homologous recombination). Preferred viral particles include, but are not limited to, adenoviruses, baculoviruses, parvoviruses, herpesviruses, poxviruses, adeno-associated viruses, Semliki Forest viruses, vaccinia viruses, and retroviruses. Preferred expression vectors include, but are not limited to, pcDNA3 (Invitrogen) and pSVL (Pharmacia Biotech). Other expression vectors include, but are not limited to, pSPORT™ vectors, pGEM™ vectors (Promega), pPROEXvectors™ (LTI, Bethesda, Md.), Bluescript™ vectors (Stratagene), PQE™ vectors (Qiagen), pSE420™ (Invitrogen), and pYES2™ (Invitrogen).

Expression constructs preferably comprise ion-x-encoding polynucleotides operatively linked to an endogenous or exogenous expression control DNA sequence and a transcription terminator. Expression control DNA sequences include promoters, enhancers, operators, and regulatory element binding sites generally, and are typically selected based on the expression systems in which the expression construct is to be utilized. Preferred promoter and enhancer sequences are generally selected for the ability to increase gene expression, while operator sequences are generally selected for the ability to regulate gene expression. Expression constructs of the invention may also include sequences encoding one or more selectable markers that permit identification of host cells bearing the construct. Expression constructs may also include sequences that facilitate, and preferably promote, homologous recombination in a host cell. Preferred constructs of the invention also include sequences necessary for replication in a host cell.

Expression constructs are preferably utilized for production of an encoded protein, but may also be utilized simply to amplify an ion-x-encoding polynucleotide sequence. In preferred embodiments, the vector is an expression vector wherein the polynucleotide of the invention is operatively linked to a polynucleotide comprising an expression control sequence. Autonomously replicating recombinant expression constructs such as plasmid and viral DNA vectors incorporating polynucleotides of the invention are also provided. Preferred expression vectors are replicable DNA constructs in which a DNA sequence encoding ion-x is operably linked or connected to suitable control sequences capable of effecting the expression of the ion-x in a suitable host. DNA regions are operably linked or connected when they are functionally related to each other. For example, a promoter is operably linked or connected to a coding sequence if it controls the transcription of the sequence. Amplification vectors do not require expression control domains, but rather need only the ability to replicate in a host, usually conferred by an origin of replication, and a selection gene to facilitate recognition of transformants. The need for control sequences in the expression vector will vary depending upon the host selected and the transformation method chosen. Generally, control sequences include a transcriptional promoter, an optional operator sequence to control transcription, a sequence encoding suitable mRNA ribosomal binding and sequences which control the termination of transcription and translation.

Preferred vectors preferably contain a promoter that is recognized by the host organism. The promoter sequences of the present invention may be prokaryotic, eukaryotic or viral. Examples of suitable prokaryotic sequences include the PR and PL promoters of bacteriophage lambda (The bacteriophage Lambda, Hershey, A. D., Ed., Cold Spring Harbor Press, Cold Spring Harbor, N.Y. (1973), which is incorporated herein by reference in its entirety; Lambda II, Hendrix, R. W., Ed., Cold Spring Harbor Press, Cold Spring Harbor, N.Y. (1980), which is incorporated herein by reference in its entirety); the trp, recA, heat shock, and lacZ promoters of E. coli and the SV40 early promoter (Benoist et al. Nature, 1981, 290, 304-310, which is incorporated herein by reference in its entirety). Additional promoters include, but are not limited to, mouse mammary tumor virus, long terminal repeat of human immunodeficiency virus, maloney virus, cytomegalovirus immediate early promoter, Epstein Barr virus, Rous sarcoma virus, human actin, human myosin, human hemoglobin, human muscle creatine, and human metalothionein.

Additional regulatory sequences can also be included in preferred vectors. Preferred examples of suitable regulatory sequences are represented by the Shine-Dalgarno of the replicase gene of the phage MS-2 and of the gene clI of bacteriophage lambda. The Shine-Dalgarno sequence may be directly followed by DNA encoding ion-x and result in the expression of the mature ion-x protein.

Moreover, suitable expression vectors can include an appropriate marker that allows the screening of the transformed host cells. The transformation of the selected host is carried out using any one of the various techniques well known to the expert in the art and described in Sambrook et al., supra.

An origin of replication can also be provided either by construction of the vector to include an exogenous origin or may be provided by the host cell chromosomal replication mechanism. If the vector is integrated into the host cell chromosome, the latter may be sufficient. Alternatively, rather than using vectors which contain viral origins of replication, one skilled in the art can transform mammalian cells by the method of co-transformation with a selectable marker and ion-x DNA. An example of a suitable marker is dihydrofolate reductase (DHFR) or thymidine kinase (see, U.S. Pat. No. 4,399,216).

Nucleotide sequences encoding ion-x may be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulation are disclosed by Sambrook et al., supra and are well known in the art. Methods for construction of mammalian expression vectors are disclosed in, for example, Okayama et al., Mol. Cell. Biol., 1983, 3, 280, Cosman et al., i Mol. Immunol., 1986, 23, 935, Cosman et al., Nature, 1984, 312, 768, EP-A-0367566, and WO 91/18982, each of which is incorporated herein by reference in its entirety.

Host Cells

According to another aspect of the invention, host cells are provided, including prokaryotic and eukaryotic cells, comprising a polynucleotide of the invention (or vector of the invention) in a manner that permits expression of the encoded ion-x polypeptide. Polynucleotides of the invention may be introduced into the host cell as part of a circular plasmid, or as linear DNA comprising an isolated protein coding region or a viral vector. Methods for introducing DNA into the host cell that are well known and routinely practiced in the art include transformation, transfection, electroporation, nuclear injection, or fusion with carriers such as liposomes, micelles, ghost cells, and protoplasts. Expression systems of the invention include bacterial, yeast, fungal, plant, insect, invertebrate, vertebrate, and mammalian cells systems.

The invention provides host cells that are transformed or transfected (stably or transiently) with polynucleotides of the invention or vectors of the invention. As stated above, such host cells are useful for amplifying the polynucleotides and also for expressing the ion-x polypeptide or fragment thereof encoded by the polynucleotide.

In still another related embodiment, the invention provides a method for producing an ion-x polypeptide (or fragment thereof) comprising the steps of growing a host cell of the invention in a nutrient medium and isolating the polypeptide or variant thereof from the cell or the medium. Because ion-x is a membrane spanning channel, it will be appreciated that, for some applications, such as certain activity assays, the preferable isolation may involve isolation of cell membranes containing the polypeptide embedded therein, whereas for other applications a more complete isolation may be preferable.

According to some aspects of the present invention, transformed host cells having an expression vector comprising any of the nucleic acid molecules described above are provided. Expression of the nucleotide sequence occurs when the expression vector is introduced into an appropriate host cell. Suitable host cells for expression of the polypeptides of the invention include, but are not limited to, prokaryotes, yeast, and eukaryotes. If a prokaryotic expression vector is employed, then the appropriate host cell would be any prokaryotic cell capable of expressing the cloned sequences. Suitable prokaryotic cells include, but are not limited to, bacteria of the genera Escherichia, Bacillus, Salmonella, Pseudomonas, Streptomyces, and Staphylococcus.

If an eukaryotic expression vector is employed, then the appropriate host cell would be any eukaryotic cell capable of expressing the cloned sequence. Preferably, eukaryotic cells are cells of higher eukaryotes. Suitable eukaryotic cells include, but are not limited to, non-human mammalian tissue culture cells and human tissue culture cells. Preferred host cells include, but are not limited to, insect cells, HeLa cells, Chinese hamster ovary cells (CHO cells), African green monkey kidney cells (COS cells), human HEK-293 cells, and murine 3T3 fibroblasts. Propagation of such cells in cell culture has become a routine procedure (see, Tissue Culture, Academic Press, Kruse and Patterson, eds. (1973), which is incorporated herein by reference in its entirety).

In addition, a yeast host may be employed as a host cell. Preferred yeast cells include, but are not limited to, the genera Saccharomyces, Pichia, and Kluveromyces. Preferred yeast hosts are S. cerevisiae and P. pastoris. Preferred yeast vectors can contain an origin of replication sequence from a 2T yeast plasmid, an autonomously replication sequence (ARS), a promoter region, sequences for polyadenylation, sequences for transcription termination, and a selectable marker gene. Shuttle vectors for replication in both yeast and E. coli are also included herein.

Alternatively, insect cells may be used as host cells. In a preferred embodiment, the polypeptides of the invention are expressed using a baculovirus expression system (see, Luckow et al., Bio/Technology, 1988, 6, 47, Baculovirus Expression Vectors: A Laboratory Manual, O'Rielly et al. (Eds.), W. H. Freeman and Company, New York, 1992, and U.S. Pat. No. 4,879,236, each of which is incorporated herein by reference in its entirety). In addition, the MAXBAC™ complete baculovirus expression system (Invitrogen) can, for example, be used for production in insect cells.

Host cells of the invention are a valuable source of immunogen for development of antibodies specifically immunoreactive with ion-x. Host cells of the invention are also useful in methods for the large-scale production of ion-x polypeptides wherein the cells are grown in a suitable culture medium and the desired polypeptide products are isolated from the cells, or from the medium in which the cells are grown, by purification methods known in the art, e.g., conventional chromatographic methods including immunoaffinity chromatography, receptor affinity chromatography, hydrophobic interaction chromatography, lectin affinity chromatography, size exclusion filtration, cation or anion exchange chromatography, high pressure liquid chromatography (HPLC), reverse phase HPLC, and the like. Still other methods of purification include those methods wherein the desired protein is expressed and purified as a fusion protein having a specific tag, label, or chelating moiety that is recognized by a specific binding partner or agent. The purified protein can be cleaved to yield the desired protein, or can be left as an intact fusion protein. Cleavage of the fusion component may produce a form of the desired protein having additional amino acid residues as a result of the cleavage process.

Knowledge of ion-x DNA sequences allows for modification of cells to permit, or increase, expression of endogenous ion-x. Cells can be modified (e.g., by homologous recombination) to provide increased expression by replacing, in whole or in part, the naturally occurring ion-x promoter with all or part of a heterologous promoter so that the cells express ion-x at higher levels. The heterologous promoter is inserted in such a manner that it is operatively linked to endogenous ion-x encoding sequences. (See, for example, PCT International Publication No. WO 94/12650, PCT International Publication No. WO 92/20808, and PCT International Publication No. WO 91/09955.) It is also contemplated that, in addition to heterologous promoter DNA, amplifiable marker DNA (e.g., ada, dhfr, and the multifunctional CAD gene which encodes carbamoyl phosphate synthase, aspartate transcarbamylase, and dihydroorotase) and/or intron DNA may be inserted along with the heterologous promoter DNA. If linked to the ion-x coding sequence, amplification of the marker DNA by standard selection methods results in co-amplification of the ion-x coding sequences in the cells.

Knock-outs

The DNA sequence information provided by the present invention also makes possible the development (e.g., by homologous recombination or “knock-out” strategies; see Capecchi, Science 244:1288-1292 (1989), which is incorporated herein by reference) of animals that fail to express functional ion-x or that express a variant of ion-x. Such animals (especially small laboratory animals such as rats, rabbits, and mice) are useful as models for studying the in vivo activities of ion-x and modulators of ion-x.

Antisense

Also made available by the invention are anti-sense polynucleotides that recognize and hybridize to polynucleotides encoding ion-x. Full-length and fragment anti-sense polynucleotides are provided. Fragment antisense molecules of the invention include (i) those that specifically recognize and hybridize to ion-x RNA (as determined by sequence comparison of DNA encoding ion-x to DNA encoding other known molecules). Identification of sequences unique to ion-x encoding polynucleotides can be deduced through use of any publicly available sequence database, and/or through use of commercially available sequence comparison programs. After identification of the desired sequences, isolation through restriction digestion or amplification using any of the various polymerase chain reaction techniques well known in the art can be performed. Anti-sense polynucleotides are particularly relevant to regulating expression of ion-x by those cells expressing ion-x mRNA.

Antisense nucleic acids (preferably 10 to 30 base-pair oligonucleotides) capable of specifically binding to ion-x expression control sequences or ion-x RNA are introduced into cells (e.g., by a viral vector or colloidal dispersion system such as a liposome). The antisense nucleic acid binds to the ion-x target nucleotide sequence in the cell and prevents transcription and/or translation of the target sequence. Phosphorothioate and methylphosphonate antisense oligonucleotides are specifically contemplated for therapeutic use by the invention. Locked nucleic acids are also specifically contemplated for therapeutic use by the present invention. (See, for example, Wahlestedt et al., Proc. Natl. Acad. Sci. USA, Vol. 97, Issue 10, 5633-5638, May 9, 2000, which is incorporated by reference in its entirety) The antisense oligonucleotides may be further modified by adding poly-L-lysine, transferrin polylysine, or cholesterol moieties at their 5′ end. Suppression of ion-x expression at either the transcriptional or translational level is useful to generate cellular or animal models for diseases/conditions characterized by aberrant ion-x expression.

Antisense oligonucleotides, or fragments of nucleotide sequences selected from the group consisting of SEQ ID NO:1 to SEQ ID NO:51, and SEQ ID NOS:103, 104, 107, and 108, or sequences complementary or homologous thereto, derived from the nucleotide sequences of the present invention encoding ion-x are useful as diagnostic tools for probing gene expression in various tissues. For example, tissue can be probed in situ with oligonucleotide probes carrying detectable groups by conventional autoradiography techniques to investigate native expression of this enzyme or pathological conditions relating thereto. Antisense oligonucleotides are preferably directed to regulatory regions of sequences selected from the group consisting of SEQ ID NO:1 to SEQ ID NO:5 1, and SEQ ID NOS:103, 104, 107, and 108, or mRNA corresponding thereto, including, but not limited to, the initiation codon, TATA box, enhancer sequences, and the like.

Transcription Factors

The ion-x sequences taught in the present invention facilitate the design of novel transcription factors for modulating ion-x expression in native cells and animals, and cells transformed or transfected with ion-x polynucleotides. For example, the Cys₂-His₂ zinc finger proteins, which bind DNA via their zinc finger domains, have been shown to be amenable to structural changes that lead to the recognition of different target sequences. These artificial zinc finger proteins recognize specific target sites with high affinity and low dissociation constants, and are able to act as gene switches to modulate gene expression. Knowledge of the particular ion-x target sequence of the present invention facilitates the engineering of zinc finger proteins specific for the target sequence using known methods such as a combination of structure-based modeling and screening of phage display libraries (Segal et al., Proc. Natl. Acad. Sci. (USA) 96:2758-2763 (1999); Liu et al., Proc. Natl. Acad. Sci. (USA) 94:5525-5530 (1997); Greisman et al., Science 275:657-661 (1997); Choo et al., J. Mol. Biol. 273:525-532 (1997)). Each zinc finger domain usually recognizes three or more base pairs. Since a recognition sequence of 18 base pairs is generally sufficient in length to render it unique in any known genome, a zinc finger protein consisting of 6 tandem repeats of zinc fingers would be expected to ensure specificity for a particular sequence (Segal et al.) The artificial zinc finger repeats, designed based on ion-x sequences, are fused to activation or repression domains to promote or suppress ion-x expression (Liu et al.) Alternatively, the zinc finger domains can be fused to the TATA box-binding factor (TBP) with varying lengths of linker region between the zinc finger peptide and the TBP to create either transcriptional activators or repressors (Kim et al., Proc. Natl. Acad. Sci. (USA) 94:3616-3620 (1997). Such proteins and polynucleotides that encode them, have utility for modulating ion-x expression in vivo in both native cells, animals and humans; and/or cells transfected with ion-x -encoding sequences. The novel transcription factor can be delivered to the target cells by transfecting constructs that express the transcription factor (gene therapy), or by introducing the protein. Engineered zinc finger proteins can also be designed to bind RNA sequences for use in therapeutics as alternatives to antisense or catalytic RNA methods (McColl et al., Proc. Natl. Acad. Sci. (USA) 96:9521-9526 (1997); Wu et al., Proc. Natl. Acad. Sci. (USA) 92:344-348 (1995)). The present invention contemplates methods of designing such transcription factors based on the gene sequence of the invention, as well as customized zinc finger proteins, that are useful to modulate ion-x expression in cells (native or transformed) whose genetic complement includes these sequences.

Polypeptides

The invention also provides purified and isolated mammalian ion-x polypeptides encoded by a polynucleotide of the invention. Presently preferred is a human ion-x polypeptide comprising the amino acid sequence set out in sequences selected from the group consisting of SEQ ID NO:52 to SEQ ID NO:102, and SEQ ID NOS: 105, 106, 109, and 110, or fragments thereof comprising an epitope specific to the polypeptide. By “epitope specific to” is meant a portion of the ion-x receptor that is recognizable by an antibody that is specific for the ion-x, as defined in detail below.

Although the sequences provided are particular human sequences, the invention is intended to include within its scope other human allelic variants; non-human mammalian forms of ion-x, and other vertebrate forms of ion-x.

It will be appreciated that extracellular epitopes are particularly useful for generating and screening for antibodies and other binding compounds that bind to receptors such as ion-x. Thus, in another preferred embodiment, the invention provides a purified and isolated polypeptide comprising at least one extracellular domain of ion-x. Purified and isolated polypeptides comprising the extracellular domain of ion-x are highly preferred. Also preferred is a purified and isolated polypeptide comprising an ion-x fragment selected from the group consisting of the extracellular domain of ion-x, a transmembrane domain of ion-x, the cytoplasmic region of ion-x, and fusions thereof. Such fragments may be continuous portions of the native receptor. However, it will also be appreciated that knowledge of the ion-x gene and protein sequences as provided herein permits recombining of various domains that are not contiguous in the native protein.

Using a FORTRAN computer program called “tmtrest.all” [Parodi et al., Comput. Appl. Biosci. 5:527-535 (1994)], ion-x was shown to contain transmembrane-spanning domains.

The invention also embraces polypeptides that have at least 99%, at least 95%, at least 90%, at least 85%, at least 80%, at least 75%, at least 70%, at least 65%, at least 60%, at least 55% or at least 50% identity and/or homology to the preferred polypeptide of the invention. Percent amino acid sequence “identity” with respect to the preferred polypeptide of the invention is defined herein as the percentage of amino acid residues in the candidate sequence that are identical with the residues in the ion-x sequence after aligning both sequences and introducing gaps, if necessary, to achieve the maximum percent sequence identity, and not considering any conservative substitutions as part of the sequence identity. Percent sequence “homology” with respect to the preferred polypeptide of the invention is defined herein as the percentage of amino acid residues in the candidate sequence that are identical with the residues in the ion-x sequence after aligning the sequences and introducing gaps, if necessary, to achieve the maximum percent sequence identity, and also considering any conservative substitutions as part of the sequence identity.

In one aspect, percent homology is calculated as the percentage of amino acid residues in the smaller of two sequences which align with identical amino acid residue in the sequence being compared, when four gaps in a length of 100 amino acids may be introduced to maximize alignment [Dayhoff, in Atlas of Protein Sequence and Structure, Vol. 5, p. 124, National Biochemical Research Foundation, Washington, D.C. (1972), incorporated herein by reference].

Polypeptides of the invention may be isolated from natural cell sources or may be chemically synthesized, but are preferably produced by recombinant procedures involving host cells of the invention. Use of mammalian host cells is expected to provide for such post-translational modifications (e.g., glycosylation, truncation, lipidation, and phosphorylation) as may be needed to confer optimal biological activity on recombinant expression products of the invention. Glycosylated and non-glycosylated forms of ion-x polypeptides are embraced by the invention.

The invention also embraces variant (or analog) ion-x polypeptides. In one example, insertion variants are provided wherein one or more amino acid residues supplement an ion-x amino acid sequence. Insertions may be located at either or both termini of the protein, or may be positioned within internal regions of the ion-x amino acid sequence. Insertional variants with additional residues at either or both termini can include, for example, fusion proteins and proteins including amino acid tags or labels.

Insertion variants include ion-x polypeptides wherein one or more amino acid residues are added to an ion-x acid sequence or to a biologically active fragment thereof.

Variant products of the invention also include mature ion-x products, i.e., ion-x products wherein leader or signal sequences are removed, with additional amino terminal residues. The additional amino terminal residues may be derived from another protein, or may include one or more residues that are not identifiable as being derived from specific proteins. Ion-x products with an additional methionine residue at position −1 (Met⁻¹-ion-x) are contemplated, as are variants with additional methionine and lysine residues at positions −2 and −1 (Met⁻²-Lys⁻²-ion-x). Variants of ion-x with additional Met, Met-Lys, Lys residues (or one or more basic residues in general) are particularly useful for enhanced recombinant protein production in bacterial host cells.

The invention also embraces ion-x variants having additional amino acid residues that result from use of specific expression systems. For example, use of commercially available vectors that express a desired polypeptide as part of a glutathione-S-transferase (GST) fusion product provides the desired polypeptide having an additional glycine residue at position −1 after cleavage of the GST component from the desired polypeptide. Variants that result from expression in other vector systems are also contemplated.

Insertional variants also include fusion proteins wherein the amino terminus and/or the carboxy terminus of ion-x is/are fused to another polypeptide.

In another aspect, the invention provides deletion variants wherein one or more amino acid residues in an ion-x polypeptide are removed. Deletions can be effected at one or both termini of the ion-x polypeptide, or with removal of one or more non-terminal amino acid residues of ion-x. Deletion variants, therefore, include all fragments of an ion-x polypeptide.

The invention also embraces polypeptide fragments of sequences selected from the group consisting of SEQ ID NO:52 to SEQ ID NO:102, and SEQ ID NOS: 105, 106, 109, and 110, wherein the fragments maintain biological (e.g., ligand binding and/or ion trafficking) and/or immunological properties of a ion-x polypeptide.

In one preferred embodiment of the invention, an isolated nucleic acid molecule comprises a nucleotide sequence that encodes a polypeptide comprising an amino acid sequence homologous to a sequence selected from the group consisting of SEQ ID NO:52 to SEQ ID NO:102, and SEQ ID NOS: 105, 106, 109, and 110, and fragments thereof, wherein the nucleic acid molecule encodes at least a portion of ion-x. In a more preferred embodiment, the isolated nucleic acid molecule comprises a sequence selected from the group consisting of SEQ ID NO:1 to SEQ ID NO:51, and SEQ ID NOS:103, 104, 107, and 108, and fragments thereof.

As used in the present invention, polypeptide fragments comprise at least 5, 10, 15, 20, 25, 30, 35, or 40 consecutive amino acids of a sequence selected from the group consisting of SEQ ID NO:52 to SEQ ID NO:102, and SEQ ID NOS: 105, 106, 109, and 110. Preferred polypeptide fragments display antigenic properties unique to, or specific for, human ion-x and its allelic and species homologs. Fragments of the invention having the desired biological and immunological properties can be prepared by any of the methods well known and routinely practiced in the art.

In one embodiment of the invention, the nucleic acid molecule comprises SEQ ID NO:1. Alternatively, the nucleic acid molecule comprises a fragment of SEQ ID NO:1. Preferably, the invention provides fragments of SEQ ID NO:1 which comprise at least 14 and preferably at least 16, 18, 20, 25, 50, or 75 consecutive nucleotides. The fragment can be located within any portion of SEQ ID NO:1, may include more than one portion of SEQ ID NO:1, or may include repeated portions of SEQ ID NO:1. In a preferred embodiment, the nucleic acid molecule comprises a sequence related to the serotonin receptor.

In another embodiment of the invention, the nucleic acid molecule comprises SEQ ID NO:2. Alternatively, the nucleic acid molecule comprises a fragment of SEQ ID NO:2. Preferably, the invention provides fragments of SEQ ID NO:2 which comprise at least 14 and preferably at least 16, 18, 20, 25, 50, or 75 consecutive nucleotides. The fragment can be located within any portion of SEQ ID NO:2, may include more than one portion of SEQ ID NO:2, or may include repeated portions of SEQ ID NO:2. In a preferred embodiment, the nucleic acid molecule comprises a sequence related to the NMDA glutamate receptor.

In yet another embodiment of the invention, the nucleic acid molecule comprises SEQ ID NO:3. Alternatively, the nucleic acid molecule comprises a fragment of SEQ ID NO:3. Preferably, the invention provides fragments of SEQ ID NO:3 which comprise at least 14 and preferably at least 16, 18, 20, 25, 50, or 75 consecutive nucleotides. The fragment can be located within any portion of SEQ ID NO:3, may include more than one portion of SEQ ID NO:3, or may include repeated portions of SEQ ID NO:3. In a preferred embodiment, the nucleic acid molecule comprises a sequence related to the NMDA glutamate receptor.

In still another embodiment of the invention, the nucleic acid molecule comprises SEQ ID NO:4. Alternatively, the nucleic acid molecule comprises a fragment of SEQ ID NO:4. Preferably, the invention provides fragments of SEQ ID NO:4 which comprise at least 14 and preferably at least 16, 18, 20, 25, 50, or 75 consecutive nucleotides. The fragment can be located within any portion of SEQ ID NO:4, may include more than one portion of SEQ ID NO:4, or may include repeated portions of SEQ ID NO:4. In a preferred embodiment, the nucleic acid molecule comprises a sequence related to the NMDA glutamate receptor.

In another embodiment of the invention, the nucleic acid molecule comprises SEQ ID NO:5. Alternatively, the nucleic acid molecule comprises a fragment of SEQ ID NO:5. Preferably, the invention provides fragments of SEQ ID NO:5 which comprise at least 14 and preferably at least 16, 18, 20, 25, 50, or 75 consecutive nucleotides. The fragment can be located within any portion of SEQ ID NO:5, may include more than one portion of SEQ ID NO:5, or may include repeated portions of SEQ ID NO:5. In a preferred embodiment, the nucleic acid molecule comprises a sequence related to the GABA receptor, rho-2 subunit precursor.

In yet another embodiment of the invention, the nucleic acid molecule comprises SEQ ID NO:6. Alternatively, the nucleic acid molecule comprises a fragment of SEQ ID NO:6. Preferably, the invention provides fragments of SEQ ID NO:6 which comprise at least 14 and preferably at least 16, 18, 20, 25, 50, or 75 consecutive nucleotides. The fragment can be located within any portion of SEQ ID NO:6, may include more than one portion of SEQ ID NO:6, or may include repeated portions of SEQ ID NO:6. In a preferred embodiment, the nucleic acid molecule comprises a sequence related to the acetylcholine receptor.

In still another embodiment of the invention, the nucleic acid molecule comprises SEQ ID NO:7. Alternatively, the nucleic acid molecule comprises a fragment of SEQ ID NO:7. Preferably, the invention provides fragments of SEQ ID NO:7 which comprise at least 14 and preferably at least 16, 18, 20, 25, 50, or 75 consecutive nucleotides. The fragment can be located within any portion of SEQ ID NO:7, may include more than one portion of SEQ ID NO:7, or may include repeated portions of SEQ ID NO:7. In a preferred embodiment, the nucleic acid molecule comprises a sequence related to the acetylcholine receptor, alpha-9 chain precursor.

In one embodiment of the invention, the nucleic acid molecule comprises SEQ ID NO:8. Alternatively, the nucleic acid molecule comprises a fragment of SEQ ID NO:8. Preferably, the invention provides fragments of SEQ ID NO:8 which comprise at least 14 and preferably at least 16, 18, 20, 25, 50, or 75 consecutive nucleotides. The fragment can be located within any portion of SEQ ID NO:8, may include more than one portion of SEQ ID NO:8, or may include repeated portions of SEQ ID NO:8. In a preferred embodiment, the nucleic acid molecule comprises a sequence related to the acetylcholine receptor.

In another embodiment of the invention, the nucleic acid molecule comprises SEQ ID NO:9. Alternatively, the nucleic acid molecule comprises a fragment of SEQ ID NO:9. Preferably, the invention provides fragments of SEQ ID NO:9 which comprise at least 14 and preferably at least 16, 18, 20, 25, 50, or 75 consecutive nucleotides. The fragment can be located within any portion of SEQ ID NO:9, may include more than one portion of SEQ ID NO:9, or may include repeated portions of SEQ ID NO:9. In a preferred embodiment, the nucleic acid molecule comprises a sequence related to the acetylcholine receptor.

In yet another embodiment of the invention, the nucleic acid molecule comprises SEQ ID NO:10. Alternatively, the nucleic acid molecule comprises a fragment of SEQ ID NO:10. Preferably, the invention provides fragments of SEQ ID NO:10 which comprise at least 14 and preferably at least 16, 18, 20, 25, 50, or 75 consecutive nucleotides. The fragment can be located within any portion of SEQ ID NO:10, may include more than one portion of SEQ ID NO:10, or may include repeated portions of SEQ ID NO:10. In a preferred embodiment, the nucleic acid molecule comprises a sequence related to the GABA receptor, alpha-1 subunit precursor.

In still another embodiment of the invention, the nucleic acid molecule comprises SEQ ID NO:11. Alternatively, the nucleic acid molecule comprises a fragment of SEQ ID NO:11. Preferably, the invention provides fragments of SEQ ID NO:11 which comprise at least 14 and preferably at least 16, 18, 20, 25, 50, or 75 consecutive nucleotides. The fragment can be located within any portion of SEQ ID NO:11, may include more than one portion of SEQ ID NO:11, or may include repeated portions of SEQ ID NO:11. In a preferred embodiment, the nucleic acid molecule comprises a sequence related to the glutamate receptor, ionotropic kainate 4 precursor.

In another embodiment of the invention, the nucleic acid molecule comprises SEQ ID NO:12. Alternatively, the nucleic acid molecule comprises a fragment of SEQ ID NO:12. Preferably, the invention provides fragments of SEQ ID NO:12 which comprise at least 14 and preferably at least 16, 18, 20, 25, 50, or 75 consecutive nucleotides. The fragment can be located within any portion of SEQ ID NO:12, may include more than one portion of SEQ ID NO:12, or may include repeated portions of SEQ ID NO:12. In a preferred embodiment, the nucleic acid molecule comprises a sequence related to the acetylcholine receptor, alpha-la chain precursor.

In yet another embodiment of the invention, the nucleic acid molecule comprises SEQ ID NO:13. Alternatively, the nucleic acid molecule comprises a fragment of SEQ ID NO:13. Preferably, the invention provides fragments of SEQ ID NO:13 which comprise at least 14 and preferably at least 16, 18, 20, 25, 50, or 75 consecutive nucleotides. The fragment can be located within any portion of SEQ ID NO:13, may include more than one portion of SEQ ID NO:13, or may include repeated portions of SEQ ID NO:13. In a preferred embodiment, the nucleic acid molecule comprises a sequence related to the acetylcholine receptor, alpha-3 chain precursor.

In still another embodiment of the invention, the nucleic acid molecule comprises SEQ ID NO:14. Alternatively, the nucleic acid molecule comprises a fragment of SEQ ID NO:14. Preferably, the invention provides fragments of SEQ ID NO:14 which comprise at least 14 and preferably at least 16, 18, 20, 25, 50, or 75 consecutive nucleotides. The fragment can be located within any portion of SEQ ID NO:14, may include more than one portion of SEQ ID NO:14, or may include repeated portions of SEQ ID NO:14. In a preferred embodiment, the nucleic acid molecule comprises a sequence related to the acetylcholine receptor, alpha-4 chain precursor.

In one embodiment of the invention, the nucleic acid molecule comprises SEQ ID NO:15. Alternatively, the nucleic acid molecule comprises a fragment of SEQ ID NO:15. Preferably, the invention provides fragments of SEQ ID NO:15 which comprise at least 14 and preferably at least 16, 18, 20, 25, 50, or 75 consecutive nucleotides. The fragment can be located within any portion of SEQ ID NO:15, may include more than one portion of SEQ ID NO:15, or may include repeated portions of SEQ ID NO:15. In a preferred embodiment, the nucleic acid molecule comprises a sequence related to the acetylcholine receptor, alpha-7 chain precursor.

In another embodiment of the invention, the nucleic acid molecule comprises SEQ ID NO:16. Alternatively, the nucleic acid molecule comprises a fragment of SEQ ID NO:16. Preferably, the invention provides fragments of SEQ ID NO:16 which comprise at least 14 and preferably at least 16, 18, 20, 25, 50, or 75 consecutive nucleotides. The fragment can be located within any portion of SEQ ID NO:16, may include more than one portion of SEQ ID NO:16, or may include repeated portions of SEQ ID NO:16. In a preferred embodiment, the nucleic acid molecule comprises a sequence related to the acetylcholine receptor, alpha-9 chain precursor.

In yet another embodiment of the invention, the nucleic acid molecule comprises SEQ ID NO:17. Alternatively, the nucleic acid molecule comprises a fragment of SEQ ID NO:17. Preferably, the invention provides fragments of SEQ ID NO:17 which comprise at least 14 and preferably at least 16, 18, 20, 25, 50, or 75 consecutive nucleotides. The fragment can be located within any portion of SEQ ID NO:17, may include more than one portion of SEQ ID NO:17, or may include repeated portions of SEQ ID NO:17. In a preferred embodiment, the nucleic acid molecule comprises a sequence related to the acetylcholine receptor, alpha-9 chain precursor.

In still another embodiment of the invention, the nucleic acid molecule comprises SEQ ID NO:18. Alternatively, the nucleic acid molecule comprises a fragment of SEQ ID NO:18. Preferably, the invention provides fragments of SEQ ID NO:18 which comprise at least 14 and preferably at least 16, 18, 20, 25, 50, or 75 consecutive nucleotides. The fragment can be located within any portion of SEQ ID NO:18, may include more than one portion of SEQ ID NO:18, or may include repeated portions of SEQ ID NO:18. In a preferred embodiment, the nucleic acid molecule comprises a sequence related to the acetylcholine receptor, alpha-9 chain precursor.

In another embodiment of the invention, the nucleic acid molecule comprises SEQ ID NO:19. Alternatively, the nucleic acid molecule comprises a fragment of SEQ ID NO:19. Preferably, the invention provides fragments of SEQ ID NO:19 which comprise at least 14 and preferably at least 16, 18, 20, 25, 50, or 75 consecutive nucleotides. The fragment can be located within any portion of SEQ ID NO:19, may include more than one portion of SEQ ID NO:19, or may include repeated portions of SEQ ID NO:19. In a preferred embodiment, the nucleic acid molecule comprises a sequence related to the acetylcholine receptor, alpha-9 chain precursor.

In yet another embodiment of the invention, the nucleic acid molecule comprises SEQ ID NO:20. Alternatively, the nucleic acid molecule comprises a fragment of SEQ ID NO:20. Preferably, the invention provides fragments of SEQ ID NO:20 which comprise at least 14 and preferably at least 16, 18, 20, 25, 50, or 75 consecutive nucleotides. The fragment can be located within any portion of SEQ ID NO:20, may include more than one portion of SEQ ID NO:20, or may include repeated portions of SEQ ID NO:20. In a preferred embodiment, the nucleic acid molecule comprises a sequence related to the acetylcholine receptor, alpha-9 chain precursor.

In still another embodiment of the invention, the nucleic acid molecule comprises SEQ ID NO:21. Alternatively, the nucleic acid molecule comprises a fragment of SEQ ID NO:21. Preferably, the invention provides fragments of SEQ ID NO:21 which comprise at least 14 and preferably at least 16, 18, 20, 25, 50, or 75 consecutive nucleotides. The fragment can be located within any portion of SEQ ID NO:21, may include more than one portion of SEQ ID NO:21, or may include repeated portions of SEQ ID NO:21. In a preferred embodiment, the nucleic acid molecule comprises a sequence related to the acetylcholine receptor, alpha chain precursor.

In one embodiment of the invention, the nucleic acid molecule comprises SEQ ID NO:22. Alternatively, the nucleic acid molecule comprises a fragment of SEQ ID NO:22. Preferably, the invention provides fragments of SEQ ID NO:22 which comprise at least 14 and preferably at least 16, 18, 20, 25, 50, or 75 consecutive nucleotides. The fragment can be located within any portion of SEQ ID NO:22, may include more than one portion of SEQ ID NO:22, or may include repeated portions of SEQ ID NO:22. In a preferred embodiment, the nucleic acid molecule comprises a sequence related to the acetylcholine receptor, alpha chain precursor.

In another embodiment of the invention, the nucleic acid molecule comprises SEQ NO:23. Alternatively, the nucleic acid molecule comprises a fragment of SEQ ID NO:23. Preferably, the invention provides fragments of SEQ ID NO:23 which comprise at least 14 and preferably at least 16, 18, 20, 25, 50, or 75 consecutive nucleotides. The fragment can be located within any portion of SEQ ID NO:23, may include more than one portion of SEQ ID NO:23, or may include repeated portions of SEQ ID NO:23. In a preferred embodiment, the nucleic acid molecule comprises a sequence related to the acetylcholine receptor, alpha chain precursor.

In yet another embodiment of the invention, the nucleic acid molecule comprises SEQ ID NO:24. Alternatively, the nucleic acid molecule comprises a fragment of SEQ ID NO:24. Preferably, the invention provides fragments of SEQ ID NO:24 which comprise at least 14 and preferably at least 16, 18, 20, 25, 50, or 75 consecutive nucleotides. The fragment can be located within any portion of SEQ ID NO:24, may include more than one portion of SEQ ID NO:24, or may include repeated portions of SEQ ID NO:24. In a preferred embodiment, the nucleic acid molecule comprises a sequence related to the acetylcholine receptor, alpha chain precursor.

In still another embodiment of the invention, the nucleic acid molecule comprises SEQ ID NO:25. Alternatively, the nucleic acid molecule comprises a fragment of SEQ ID NO:25. Preferably, the invention provides fragments of SEQ i) NO:25 which comprise at least 14 and preferably at least 16, 18, 20, 25, 50, or 75 consecutive nucleotides. The fragment can be located within any portion of SEQ ID NO:25, may include more than one portion of SEQ ID NO:25, or may include repeated portions of SEQ ID NO:25. In a preferred embodiment, the nucleic acid molecule comprises a sequence related to the acetylcholine receptor, beta chain precursor.

In another embodiment of the invention, the nucleic acid molecule comprises SEQ ID NO:26. Alternatively, the nucleic acid molecule comprises a fragment of SEQ ID NO:26. Preferably, the invention provides fragments of SEQ ID NO:26 which comprise at least 14 and preferably at least 16, 18, 20, 25, 50, or 75 consecutive nucleotides. The fragment can be located within any portion of SEQ ID NO:26, may include more than one portion of SEQ ID NO:26, or may include repeated portions of SEQ ID NO:26. In a preferred embodiment, the nucleic acid molecule comprises a sequence related to the acetylcholine receptor, beta chain precursor.

In yet another embodiment of the invention, the nucleic acid molecule comprises SEQ ID NO:27. Alternatively, the nucleic acid molecule comprises a fragment of SEQ ID NO:27. Preferably, the invention provides fragments of SEQ ID NO:27 which comprise at least 14 and preferably at least 16, 18, 20, 25, 50, or 75 consecutive nucleotides. The fragment can be located within any portion of SEQ ID NO:27, may include more than one portion of SEQ ID NO:27, or may include repeated portions of SEQ ID NO:27. In a preferred embodiment, the nucleic acid molecule comprises a sequence related to the acetylcholine receptor, gamma chain precursor.

In still another embodiment of the invention, the nucleic acid molecule comprises SEQ ID NO:28. Alternatively, the nucleic acid molecule comprises a fragment of SEQ ID NO:28. Preferably, the invention provides fragments of SEQ ID NO:28 which comprise at least 14 and preferably at least 16, 18, 20, 25, 50, or 75 consecutive nucleotides. The fragment can be located within any portion of SEQ ID NO:28, may include more than one portion of SEQ ID NO:28, or may include repeated portions of SEQ ID NO:28. In a preferred embodiment, the nucleic acid molecule comprises a sequence related to the acetylcholine receptor, beta-3 chain precursor.

In one embodiment of the invention, the nucleic acid molecule comprises SEQ ID NO:29. Alternatively, the nucleic acid molecule comprises a fragment of SEQ ID NO:29. Preferably, the invention provides fragments of SEQ ID NO:29 which comprise at least 14 and preferably at least 16, 18, 20, 25, 50, or 75 consecutive nucleotides. The fragment can be located within any portion of SEQ ID NO:29, may include more than one portion of SEQ ID NO:29, or may include repeated portions of SEQ ID NO:29. In a preferred embodiment, the nucleic acid molecule comprises a sequence related to the acetylcholine receptor, beta-4 chain precursor (non alpha-2).

In another embodiment of the invention, the nucleic acid molecule comprises SEQ ID NO:30. Alternatively, the nucleic acid molecule comprises a fragment of SEQ ID NO:30. Preferably, the invention provides fragments of SEQ ID NO:30 which comprise at least 14 and preferably at least 16, 18, 20, 25, 50, or 75 consecutive nucleotides. The fragment can be located within any portion of SEQ ID NO:30, may include more than one portion of SEQ ID NO:30, or may include repeated portions of SEQ ID NO:30. In a preferred embodiment, the nucleic acid molecule comprises a sequence related to the acetylcholine receptor, beta-4 chain precursor (non alpha-2).

In yet another embodiment of the invention, the nucleic acid molecule comprises SEQ ID NO:31. Alternatively, the nucleic acid molecule comprises a fragment of SEQ ID NO:31. Preferably, the invention provides fragments of SEQ ID NO:31 which comprise at least 14 and preferably at least 16, 18, 20, 25, 50, or 75 consecutive nucleotides. The fragment can be located within any portion of SEQ ID NO:31, may include more than one portion of SEQ ID NO:31, or may include repeated portions of SEQ ID NO:31. In a preferred embodiment, the nucleic acid molecule comprises a sequence related to the acetylcholine receptor, beta-4 chain precursor (non alpha-2).

In still another embodiment of the invention, the nucleic acid molecule comprises SEQ ID NO:32. Alternatively, the nucleic acid molecule comprises a fragment of SEQ ID NO:32. Preferably, the invention provides fragments of SEQ ID NO:32 which comprise at least 14 and preferably at least 16, 18, 20, 25, 50, or 75 consecutive nucleotides. The fragment can be located within any portion of SEQ ID NO:32, may include more than one portion of SEQ ID NO:32, or may include repeated portions of SEQ ID NO:32. In a preferred embodiment, the nucleic acid molecule comprises a sequence related to the acetylcholine receptor, beta-4 chain precursor (non alpha-2).

In another embodiment of the invention, the nucleic acid molecule comprises SEQ ID NO:33. Alternatively, the nucleic acid molecule comprises a fragment of SEQ ID NO:33. Preferably, the invention provides fragments of SEQ ID NO:33 which comprise at least 14 and preferably at least 16, 18, 20, 25, 50, or 75 consecutive nucleotides. The fragment can be located within any portion of SEQ ID NO:33, may include more than one portion of SEQ ID NO:33, or may include repeated portions of SEQ ID NO:33. In a preferred embodiment, the nucleic acid molecule comprises a sequence related to the acetylcholine receptor, beta-4 chain precursor (non alpha-2).

In yet another embodiment of the invention, the nucleic acid molecule comprises SEQ ID NO:34. Alternatively, the nucleic acid molecule comprises a fragment of SEQ ID NO:34. Preferably, the invention provides fragments of SEQ ID NO:34 which comprise at least 14 and preferably at least 16, 18, 20, 25, 50, or 75 consecutive nucleotides. The fragment can be located within any portion of SEQ ID NO:34, may include more than one portion of SEQ ID NO:34, or may include repeated portions of SEQ ID NO:34. In a preferred embodiment, the nucleic acid molecule comprises a sequence related to the acetylcholine receptor, non-alpha chain precursor.

In still another embodiment of the invention, the nucleic acid molecule comprises SEQ ID NO:35. Alternatively, the nucleic acid molecule comprises a fragment of SEQ ID NO:35. Preferably, the invention provides fragments of SEQ ID NO:35 which comprise at least 14 and preferably at least 16, 18, 20, 25, 50, or 75 consecutive nucleotides. The fragment can be located within any portion of SEQ ID NO:35, may include more than one portion of SEQ ID NO:35, or may include repeated portions of SEQ ID NO:35. In a preferred embodiment, the nucleic acid molecule comprises a sequence related to the GABA receptor, pi subunit precursor.

In one embodiment of the invention, the nucleic acid molecule comprises SEQ ID NO:36. Alternatively, the nucleic acid molecule comprises a fragment of SEQ ID NO:36. Preferably, the invention provides fragments of SEQ ID NO:36 which comprise at least 14 and preferably at least 16, 18, 20, 25, 50, or 75 consecutive nucleotides. The fragment can be located within any portion of SEQ ID NO:36, may include more than one portion of SEQ ID NO:36, or may include repeated portions of SEQ ID NO:36. In a preferred embodiment, the nucleic acid molecule comprises a sequence related to the GABA receptor, beta-1 subunit precursor.

In another embodiment of the invention, the nucleic acid molecule comprises SEQ ID NO:37. Alternatively, the nucleic acid molecule comprises a fragment of SEQ ID NO:37. Preferably, the invention provides fragments of SEQ ID NO:37 which comprise at least 14 and preferably at least 16, 18, 20, 25, 50, or 75 consecutive nucleotides. The fragment can be located within any portion of SEQ ID NO:37, may include more than one portion of SEQ ID NO:37, or may include repeated portions of SEQ ID NO:37. In a preferred embodiment, the nucleic acid molecule comprises a sequence related to the GABA receptor, beta-3 subunit precursor.

In yet another embodiment of the invention, the nucleic acid molecule comprises SEQ ID NO:38. Alternatively, the nucleic acid molecule comprises a fragment of SEQ ID NO:38. Preferably, the invention provides fragments of SEQ ID NO:38 which comprise at least 14 and preferably at least 16, 18, 20, 25, 50, or 75 consecutive nucleotides. The fragment can be located within any portion of SEQ ID NO:38, may include more than one portion of SEQ ID NO:38, or may include repeated portions of SEQ ID NO:38. In a preferred embodiment, the nucleic acid molecule comprises a sequence related to the GABA receptor, gamma-1 subunit precursor.

In still another embodiment of the invention, the nucleic acid molecule comprises SEQ ID NO:39. Alternatively, the nucleic acid molecule comprises a fragment of SEQ ID NO:39. Preferably, the invention provides fragments of SEQ ID NO:39 which comprise at least 14 and preferably at least 16, 18, 20, 25, 50, or 75 consecutive nucleotides. The fragment can be located within any portion of SEQ ID NO:39, may include more than one portion of SEQ ID NO:39, or may include repeated portions of SEQ ID NO:39. In a preferred embodiment, the nucleic acid molecule comprises a sequence related to the GABA receptor, delta subunit precursor.

In another embodiment of the invention, the nucleic acid molecule comprises SEQ ID NO:40. Alternatively, the nucleic acid molecule comprises a fragment of SEQ ID NO:40. Preferably, the invention provides fragments of SEQ ID NO:40 which comprise at least 14 and preferably at least 16, 18, 20, 25, 50, or 75 consecutive nucleotides. The fragment can be located within any portion of SEQ ID NO:40, may include more than one portion of SEQ ID NO:40, or may include repeated portions of SEQ ID NO:40. In a preferred embodiment, the nucleic acid molecule comprises a sequence related to the GABA receptor, rho-2 subunit precursor.

In yet another embodiment of the invention, the nucleic acid molecule comprises SEQ ID NO:41. Alternatively, the nucleic acid molecule comprises a fragment of SEQ ID NO:41. Preferably, the invention provides fragments of SEQ ID NO:41 which comprise at least 14 and preferably at least 16, 18, 20, 25, 50, or 75 consecutive nucleotides. The fragment can be located within any portion of SEQ ID NO:41, may include more than one portion of SEQ ID NO:41, or may include repeated portions of SEQ ID NO:41. In a preferred embodiment, the nucleic acid molecule comprises a sequence related to the GABA receptor, rho-2 subunit precursor.

In still another embodiment of the invention, the nucleic acid molecule comprises SEQ ID NO:42. Alternatively, the nucleic acid molecule comprises a fragment of SEQ ID NO:42. Preferably, the invention provides fragments of SEQ ID NO:42 which comprise at least 14 and preferably at least 16, 18, 20, 25, 50, or 75 consecutive nucleotides. The fragment can be located within any portion of SEQ ID NO:42, may include more than one portion of SEQ ID NO:42, or may include repeated portions of SEQ ID NO:42. In a preferred embodiment, the nucleic acid molecule comprises a sequence related to the glutamate receptor, ionotropic kainate 5 precursor (glutamate receptor ka-2).

In one embodiment of the invention, the nucleic acid molecule comprises SEQ ID NO:43. Alternatively, the nucleic acid molecule comprises a fragment of SEQ ID NO:43. Preferably, the invention provides fragments of SEQ ID NO:43 which comprise at least 14 and preferably at least 16, 18, 20, 25, 50, or 75 consecutive nucleotides. The fragment can be located within any portion of SEQ ID NO:43, may include more than one portion of SEQ ID NO:43, or may include repeated portions of SEQ ID NO:43. In a preferred embodiment, the nucleic acid molecule comprises a sequence related to the glutamate receptor, ionotropic kainate 5 precursor (glutamate receptor ka-2).

In another embodiment of the invention, the nucleic acid molecule comprises SEQ ID NO:44. Alternatively, the nucleic acid molecule comprises a fragment of SEQ ID NO:44. Preferably, the invention provides fragments of SEQ ID NO:44 which comprise at least 14 and preferably at least 16, 18, 20, 25, 50, or 75 consecutive nucleotides. The fragment can be located within any portion of SEQ ID NO:44, may include more than one portion of SEQ ID NO:44, or may include repeated portions of SEQ ID NO:44. In a preferred embodiment, the nucleic acid molecule comprises a sequence related to the glutamate receptor precursor (kainate-binding protein).

In yet another embodiment of the invention, the nucleic acid molecule comprises SEQ ID NO:45. Alternatively, the nucleic acid molecule comprises a fragment of SEQ ID NO:45. Preferably, the invention provides fragments of SEQ ID NO:45 which comprise at least 14 and preferably at least 16, 18, 20, 25, 50, or 75 consecutive nucleotides. The fragment can be located within any portion of SEQ ID NO:45, may include more than one portion of SEQ ID NO:45, or may include repeated portions of SEQ ID NO:45. In a preferred embodiment, the nucleic acid molecule comprises a sequence related to the glutamate receptor precursor.

In still another embodiment of the invention, the nucleic acid molecule comprises SEQ ID NO:46. Alternatively, the nucleic acid molecule comprises a fragment of SEQ ID NO:46. Preferably, the invention provides fragments of SEQ ID NO:46 which comprise at least 14 and preferably at least 16, 18, 20, 25, 50, or 75 consecutive nucleotides. The fragment can be located within any portion of SEQ iD NO:46, may include more than one portion of SEQ ID NO:46, or may include repeated portions of SEQ ID NO:46. In a preferred embodiment, the nucleic acid molecule comprises a sequence related to the G protein pathway suppressor 2 (gps2) protein.

In another embodiment of the invention, the nucleic acid molecule comprises SEQ ID NO:47. Alternatively, the nucleic acid molecule comprises a fragment of SEQ ID NO:47. Preferably, the invention provides fragments of SEQ ID NO:47 which comprise at least 14 and preferably at least 16, 18, 20, 25, 50, or 75 consecutive nucleotides. The fragment can be located within any portion of SEQ ID NO:47, may include more than one portion of SEQ ID NO:47, or may include repeated portions of SEQ ID NO:47. In a preferred embodiment, the nucleic acid molecule comprises a sequence related to the G protein pathway suppressor 2 (gps2) protein.

In yet another embodiment of the invention, the nucleic acid molecule comprises SEQ ID NO:48. Alternatively, the nucleic acid molecule comprises a fragment of SEQ ID NO:48. Preferably, the invention provides fragments of SEQ ID NO:48 which comprise at least 14 and preferably at least 16, 18, 20, 25, 50, or 75 consecutive nucleotides. The fragment can be located within any portion of SEQ ID NO:48, may include more than one portion of SEQ ID NO:48, or may include repeated portions of SEQ ID NO:48. In a preferred embodiment, the nucleic acid molecule comprises a sequence related to the glycine receptor, alpha-1 chain precursor (strychnine binding subunit).

In still another embodiment of the invention, the nucleic acid molecule comprises SEQ ID NO:49. Alternatively, the nucleic acid molecule comprises a fragment of SEQ ID NO:49. Preferably, the invention provides fragments of SEQ ID NO:49 which comprise at least 14 and preferably at least 16, 18, 20, 25, 50, or 75 consecutive nucleotides. The fragment can be located within any portion of SEQ ID NO:49, may include more than one portion of SEQ ID NO:49, or may include repeated portions of SEQ ID NO:49. In a preferred embodiment, the nucleic acid molecule comprises a sequence related to the glycine receptor, alpha-2 chain precursor.

In one embodiment of the invention, the nucleic acid molecule comprises SEQ ID NO:50. Alternatively, the nucleic acid molecule comprises a fragment of SEQ ID NO:50. Preferably, the invention provides fragments of SEQ ID NO:50 which comprise at least 14 and preferably at least 16, 18, 20, 25, 50, or 75 consecutive nucleotides. The fragment can be located within any portion of SEQ ID NO:50, may include more than one portion of SEQ ID NO:50, or may include repeated portions of SEQ ID NO:50. In a preferred embodiment, the nucleic acid molecule comprises a sequence related to the glycine receptor, alpha-2 chain precursor.

In another embodiment of the invention, the nucleic acid molecule comprises SEQ ID NO:5 1. Alternatively, the nucleic acid molecule comprises a fragment of SEQ ID NO:51. Preferably, the invention provides fragments of SEQ ID NO:51 which comprise at least 14 and preferably at least 16, 18, 20, 25, 50, or 75 consecutive nucleotides. The fragment can be located within any portion of SEQ ID NO:51, may include more than one portion of SEQ ID NO:51, or may include repeated portions of SEQ ID NO:51. In a preferred embodiment, the nucleic acid molecule comprises a sequence related to the glycine receptor, beta chain precursor.

In another embodiment of the invention, the nucleic acid molecule comprises SEQ ID NO:107. Alternatively, the nucleic acid molecule comprises a fragment of SEQ ID NO:107. Preferably, the invention provides fragments of SEQ ID NO:107 which comprise at least 14 and preferably at least 16, 18, 20, 25, 50, or 75 consecutive nucleotides. The fragment can be located within any portion of SEQ ID NO:107, may include more than one portion of SEQ ID NO:107, or may include repeated portions of SEQ ID NO:107. In a preferred embodiment, the nucleic acid molecule comprises a sequence related to the serotonin receptor.

In still another embodiment of the invention, the nucleic acid molecule comprises SEQ ID NO:108. Alternatively, the nucleic acid molecule comprises a fragment of SEQ ID NO:108. Preferably, the invention provides fragments of SEQ ID NO:108 which comprise at least 14 and preferably at least 16, 18, 20, 25, 50, or 75 consecutive nucleotides. The fragment can be located within any portion of SEQ ID NO:108, may include more than one portion of SEQ ID NO:108, or may include repeated portions of SEQ ID NO:108. In a preferred embodiment, the nucleic acid molecule comprises a sequence related to the serotonin receptor.

In still another aspect, the invention provides substitution variants of ion-x polypeptides. Substitution variants include those polypeptides wherein one or more amino acid residues of an ion-x polypeptide are removed and replaced with alternative residues. In one aspect, the substitutions are conservative in nature; however, the invention embraces substitutions that are also non-conservative. Conservative substitutions for this purpose may be defined as set out in Tables 2, 3, or 4 below.

Variant polypeptides include those wherein conservative substitutions have been introduced by modification of polynucleotides encoding polypeptides of the invention. Amino acids can be classified according to physical properties and contribution to secondary and tertiary protein structure. A conservative substitution is recognized in the art as a substitution of one amino acid for another amino acid that has similar properties. Exemplary conservative substitutions are set out in Table 2 (from WO 97/09433, page 10, published Mar. 13, 1997 (PCT/GB96/02197, filed Sep. 6, 1996), immediately below.

TABLE 2
Conservative Substitutions I

| SIDE CHAIN | |
|---|---|
| CHARACTERISTIC | AMINO ACID |
| Aliphatic | |
| Non-polar | G A P |
| | I L V |
| Polar - uncharged | C S T M |
| | N Q |
| Polar - charged | D E |
| | K R |
| Aromatic | H F W Y |
| Other | N Q D E |

Alternatively, conservative amino acids can be grouped as described in Lehninger, [Biochemistry, Second Edition; Worth Publishers, Inc. NY, N.Y. (1975), pp.71-77] as set out in Table 3, below.

TABLE 3
Conservative Substitutions II

| SIDE CHAIN | |
|---|---|
| CHARACTERISTIC | AMINO ACID |
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

As still another alternative, exemplary conservative substitutions are set out in Table 4, below.

TABLE 4
Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

It should be understood that the definition of polypeptides of the invention is intended to include polypeptides bearing modifications other than insertion, deletion, or substitution of amino acid residues. By way of example, the modifications may be covalent in nature, and include for example, chemical bonding with polymers, lipids, other organic, and inorganic moieties. Such derivatives may be prepared to increase circulating half-life of a polypeptide, or may be designed to improve the targeting capacity of the polypeptide for desired cells, tissues, or organs. Similarly, the invention further embraces ion-x polypeptides that have been covalently modified to include one or more water-soluble polymer attachments such as polyethylene glycol, polyoxyethylene glycol, or polypropylene glycol. Variants that display ligand binding properties of native ion-x and are expressed at higher levels, as well as variants that provide for constitutively active receptors, are particularly useful in assays of the invention; the variants are also useful in providing cellular, tissue and animal models of diseases/conditions characterized by aberrant ion-x activity.

In a related embodiment, the present invention provides compositions comprising purified polypeptides of the invention. Preferred compositions comprise, in addition to the polypeptide of the invention, a pharmaceutically acceptable (i.e., sterile and non-toxic) liquid, semisolid, or solid diluent that serves as a pharmaceutical vehicle, excipient, or medium. Any diluent known in the art may be used. Exemplary diluents include, but are not limited to, water, saline solutions, polyoxyethylene sorbitan monolaurate, magnesium stearate, methyl- and propylhydroxybenzoate, talc, alginates, starches, lactose, sucrose, dextrose, sorbitol, mannitol, glycerol, calcium phosphate, mineral oil, and cocoa butter.

Variants that display ligand binding properties of native ion-x and are expressed at higher levels, as well as variants that provide for constitutively active receptors, are particularly useful in assays of the invention; the variants are also useful in assays of the invention and in providing cellular, tissue and animal models of diseases/conditions characterized by aberrant ion-x activity.

Antibodies

Also comprehended by the present invention are antibodies (e.g., monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR sequences which specifically recognize a polypeptide of the invention) specific for ion-x or fragments thereof. Preferred antibodies of the invention are human antibodies that are produced and identified according to methods described in WO93/11236, published Jun. 20, 1993, which is incorporated herein by reference in its entirety. Antibody fragments, including Fab, Fab′, F(ab′)₂, and Fᵥ, are also provided by the invention. The term “specific for,” when used to describe antibodies of the invention, indicates that the variable regions of the antibodies of the invention recognize and bind ion-x polypeptides exclusively (i.e., are able to distinguish ion-x polypeptides from other known ion channel polypeptides by virtue of measurable differences in binding affinity, despite the possible existence of localized sequence identity, homology, or similarity between ion-x and such polypeptides). It will be understood that specific antibodies may also interact with other proteins (for example, S. aureus protein A or other antibodies in ELISA techniques) through interactions with sequences outside the variable region of the antibodies, and, in particular, in the constant region of the molecule. Screening assays to determine binding specificity of an antibody of the invention are well known and routinely practiced in the art. For a comprehensive discussion of such assays, see Harlow et al. (Eds.), Antibodies A Laboratory Manual; Cold Spring Harbor Laboratory; Cold Spring Harbor, N.Y. (1988), Chapter 6. Antibodies that recognize and bind fragments of the ion-x polypeptides of the invention are also contemplated, provided that the antibodies are specific for ion-x polypeptides. Antibodies of the invention can be produced using any method well known and routinely practiced in the art.

The invention provides an antibody that is specific for the ion-x of the invention. Antibody specificity is described in greater detail below. However, it should be emphasized that antibodies that can be generated from polypeptides that have previously been described in the literature and that are capable of fortuitously cross-reacting with ion-x (e.g., due to the fortuitous existence of a similar epitope in both polypeptides) are considered “cross-reactive” antibodies. Such cross-reactive antibodies are not antibodies that are “specific” for ion-x. The determination of whether an antibody is specific for ion-x or is cross-reactive with another known receptor is made using any of several assays, such as Western blotting assays, that are well known in the art. For identifying cells that express ion-x and also for modulating ion-x -ligand binding activity, antibodies that specifically bind to an extracellular epitope of the ion-x are preferred.

In one preferred variation, the invention provides monoclonal antibodies. Hybridomas that produce such antibodies also are intended as aspects of the invention. In yet another variation, the invention provides a humanized antibody. Humanized antibodies are useful for in vivo therapeutic indications.

In another variation, the invention provides a cell-free composition comprising polyclonal antibodies, wherein at least one of the antibodies is an antibody of the invention specific for ion-x. Antisera isolated from an animal is an exemplary composition, as is a composition comprising an antibody fraction of an antisera that has been resuspended in water or in another diluent, excipient, or carrier.

In still another related embodiment, the invention provides an anti-idiotypic antibody specific for an antibody that is specific for ion-x.

It is well known that antibodies contain relatively small antigen binding domains that can be isolated chemically or by recombinant techniques. Such domains are useful ion-x binding molecules themselves, and also may be reintroduced into human antibodies, or fused to toxins or other polypeptides. Thus, in still another embodiment, the invention provides a polypeptide comprising a fragment of an ion-x-specific antibody, wherein the fragment and the polypeptide bind to the ion-x. By way of non-limiting example, the invention provides polypeptides that are single chain antibodies and CDR-grafted antibodies.

Non-human antibodies may be humanized by any of the methods known in the art. In one method, the non-humans CDRs are inserted into a human antibody or consensus antibody framework sequence. Further changes can then be introduced into the antibody framework to modulate affinity or immunogenicity.

Antibodies of the invention are useful for, e.g., therapeutic purposes (by modulating activity of ion-x), diagnostic purposes to detect or quantitate ion-x, and purification of ion-x. Kits comprising an antibody of the invention for any of the purposes described herein are also comprehended. In general, a kit of the invention also includes a control antigen for which the antibody is immunospecific.

Compositions

Mutations in the ion-x gene that result in loss of normal function of the ion-x gene product underlie ion-x -related human disease states. The invention comprehends gene therapy to restore ion-x activity to treat those disease states. Delivery of a functional ion-x gene to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson, Nature, supplement to vol. 392, No. 6679, pp.25-20 (1998). For additional reviews of gene therapy technology see Friedmann, Science, 244: 1275-1281 (1989); Verma, Scientific American:68-84 (1990); and Miller, Nature, 357: 455-460 (1992). Alternatively, it is contemplated that in other human disease states, preventing the expression of, or inhibiting the activity of, ion-x will be useful in treating disease states. It is contemplated that antisense therapy or gene therapy could be applied to negatively regulate the expression of ion-x.

Another aspect of the present invention is directed to compositions, including pharmaceutical compositions, comprising any of the nucleic acid molecules or recombinant expression vectors described above and an acceptable carrier or diluent. Preferably, the carrier or diluent is pharmaceutically acceptable. Suitable carriers are described in the most recent edition of Remington's Pharmaceutical Sciences, A. Osol, a standard reference text in this field, which is incorporated herein by reference in its entirety. Preferred examples of such carriers or diluents include, but are not limited to, water, saline, Ringer's solution, dextrose solution, and 5% human serum albumin. Liposomes and nonaqueous vehicles such as fixed oils may also be used. The formulations are sterilized by commonly used techniques.

Also within the scope of the invention are compositions comprising polypeptides, polynucleotides, or antibodies of the invention that have been formulated with, e.g., a pharmaceutically acceptable carrier.

The invention also provides methods of using antibodies of the invention. For example, the invention provides a method for modulating ligand binding of an ion-x comprising the step of contacting the ion-x with an antibody specific for the ion-x, under conditions wherein the antibody binds the receptor.

Ion channels that may be expressed in the brain, such as ion-x, provide an indication that aberrant ion-x signaling activity may correlate with one or more neurological or psychological disorders. The invention also provides a method for treating a neurological or psychiatric disorder comprising the step of administering to a mammal in need of such treatment an amount of an antibody-like polypeptide of the invention that is sufficient to modulate ligand binding to an ion-x in neurons of the mammal. Ion-x may also be expressed in many tissues, including but not limited to, kidney, colon, small intestine, stomach, testis, placenta, adrenal gland, peripheral blood leukocytes, bone marrow, retina, ovary, fetal brain, fetal liver, heart, spleen, liver, lung, muscle, thyroid gland, uterus, prostate, skin, salivary gland, and pancreas. Tissues where specific ion-x of the present invention are expressed are identified in Example 12, below. Kits The present invention is also directed to kits, including pharmaceutical kits. The kits can comprise any of the nucleic acid molecules described above, any of the polypeptides described above, or any antibody which binds to a polypeptide of the invention as described above, as well as a negative control. The kit preferably comprises additional components, such as, for example, instructions, solid support, reagents helpful for quantification, and the like.

In another aspect, the invention features methods for detection of a polypeptide in a sample as a diagnostic tool for diseases or disorders, wherein the method comprises the steps of: (a) contacting the sample with a nucleic acid probe which hybridizes under hybridization assay conditions to a nucleic acid target region of a polypeptide having a sequence selected from the group consisting of SEQ ID NO:52 to SEQ ID NO:102, and SEQ ID NOS: 105, 106, 109, and 110, said probe comprising the nucleic acid sequence encoding the polypeptide, fragments thereof, and the complements of the sequences and fragments; and (b) detecting the presence or amount of the probe:target region hybrid as an indication of the disease.

In preferred embodiments of the invention, the disease is selected from the group consisting of thyroid disorders (e.g. thyreotoxicosis, myxoedema); renal failure; inflammatory conditions (e.g., Crohn's disease); diseases related to cell differentiation and homeostasis; rheumatoid arthritis; autoimmune disorders; movement disorders; CNS disorders (e.g., pain including neuropathic pain, migraine, and other headaches; stroke; psychotic and neurological disorders, including anxiety, schizophrenia, manic depression, anxiety, generalized anxiety disorder, post-traumatic-stress disorder, depression, bipolar disorder, delirium, dementia, severe mental retardation; dyskinesias, such as Huntington's disease or Tourette's Syndrome; attention disorders including ADD and ADHD, and degenerative disorders such as Parkinson's, Alzheimer's; movement disorders, including ataxias, supranuclear palsy, etc.); infections, such as viral infections caused by HIV-1 or HIV-2; metabolic and cardiovascular diseases and disorders (e.g., type 2 diabetes, obesity, anorexia, hypotension, hypertension, thrombosis, myocardial infarction, cardiomyopathies, atherosclerosis, etc.); proliferative diseases and cancers (e.g., different cancers such as breast, colon, lung, etc., and hyperproliferative disorders such as psoriasis, prostate hyperplasia, etc.); hormonal disorders (e.g., male/female hormonal replacement, polycystic ovarian syndrome, alopecia, etc.); and sexual dysfunction, among others.

Kits may be designed to detect either expression of polynucleotides encoding these proteins or the proteins themselves in order to identify tissue as being neurological. For example, oligonucleotide hybridization kits can be provided which include a container having an oligonucleotide probe specific for the ion-x-specific DNA and optionally, containers with positive and negative controls and/or instructions. Similarly, PCR kits can be provided which include a container having primers specific for the ion-x-specific sequences, DNA and optionally, containers with size markers, positive and negative controls and/or instructions.

Hybridization conditions should be such that hybridization occurs only with the genes in the presence of other nucleic acid molecules. Under stringent hybridization conditions only highly complementary nucleic acid sequences hybridize. Preferably, such conditions prevent hybridization of nucleic acids having 1 or 2 mismatches out of 20 contiguous nucleotides. Such conditions are defined supra.

The diseases for which detection of genes in a sample could be diagnostic include diseases in which nucleic acid (DNA and/or RNA) is amplified in comparison to normal cells. By “amplification” is meant increased numbers of DNA or RNA in a cell compared with normal cells.

The diseases that could be diagnosed by detection of nucleic acid in a sample preferably include central nervous system and metabolic diseases. The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well known in the art and can be readily adapted in order to obtain a sample that is compatible with the method utilized.

Alternatively, immunoassay kits can be provided which have containers container having antibodies specific for the ion-x protein and optionally, containers with positive and negative controls and/or instructions.

Kits may also be provided useful in the identification of ion-x binding partners such as natural ligands, neurotransmitters, or modulators (agonists or antagonists). Substances useful for treatment of disorders or diseases preferably show positive results in one or more in vitro assays for an activity corresponding to treatment of the disease or disorder in question. Substances that modulate the activity of the polypeptides preferably include, but are not limited to, antisense oligonucleotides, agonists and antagonists, and inhibitors of protein kinases.

Methods of Inducing Immune Response

Another aspect of the present invention is directed to methods of inducing an immune response in a mammal against a polypeptide of the invention by administering to the mammal an amount of the polypeptide sufficient to induce an immune response. The amount will be dependent on the animal species, size of the animal, and the like but can be determined by those skilled in the art.

Methods of Identifying Ligands

The invention also provides assays to identify compounds that bind ion-x. One such assay comprises the steps of: (a) contacting a composition comprising an ion-x with a compound suspected of binding ion-x; and (b) measuring binding between the compound and ion-x. In one variation, the composition comprises a cell expressing ion-x on its surface. In another variation, isolated ion-x or cell membranes comprising ion-x are employed. The binding may be measured directly, e.g., by using a labeled compound, or may be measured indirectly by several techniques, including measuring ion trafficking of ion-x induced by the compound. Compounds identified as binding ion-x may be further tested in other assays including, but not limited to, in vivo models, in order to confirm or quantitate their activity.

Specific binding molecules, including natural ligands and synthetic compounds, can be identified or developed using isolated or recombinant ion-x products, ion-x variants, or preferably, cells expressing such products. Binding partners are useful for purifying ion-x products and detection or quantification of ion-x products in fluid and tissue samples using known immunological procedures. Binding molecules are also manifestly useful in modulating (i.e., blocking, inhibiting or stimulating) biological activities of ion-x, especially those activities involved in signal transduction.

The DNA and amino acid sequence information provided by the present invention also makes possible identification of binding partner compounds with which an ion-x polypeptide or polynucleotide will interact. Methods to identify binding partner compounds include solution assays, in vitro assays wherein ion-x polypeptides are immobilized, and cell-based assays. Identification of binding partner compounds of ion-x polypeptides provides candidates for therapeutic or prophylactic intervention in pathologies associated with ion-x normal and aberrant biological activity.

The invention includes several assay systems for identifying ion-x-binding partners. In solution assays, methods of the invention comprise the steps of (a) contacting an ion-x polypeptide with one or more candidate binding partner compounds and (b) identifying the compounds that bind to the ion-x polypeptide. Identification of the compounds that bind the ion-x polypeptide can be achieved by isolating the ion-x polypeptide/binding partner complex, and separating the binding partner compound from the ion-x polypeptide. An additional step of characterizing the physical, biological, and/or biochemical properties of the binding partner compound is also comprehended in another embodiment of the invention. In one aspect, the ion-x polypeptide/binding partner complex is isolated using an antibody immunospecific for either the ion-x polypeptide or the candidate binding partner compound.

In still other embodiments, either the ion-x polypeptide or the candidate binding partner compound comprises a label or tag that facilitates its isolation, and methods of the invention to identify binding partner compounds include a step of isolating the ion-x polypeptide/binding partner complex through interaction with the label or tag. An exemplary tag of this type is a poly-histidine sequence, generally around six histidine residues, that permits isolation of a compound so labeled using nickel chelation. Other labels and tags, such as the FLAG® tag (Eastman Kodak, Rochester, N.Y.), well known and routinely used in the art, are embraced by the invention.

In one variation of an in vitro assay, the invention provides a method comprising the steps of (a) contacting an immobilized ion-x polypeptide with a candidate binding partner compound and (b) detecting binding of the candidate compound to the ion-x polypeptide. In an alternative embodiment, the candidate binding partner compound is immobilized and binding of ion-x is detected. Immobilization is accomplished using any of the methods well known in the art, including covalent bonding to a support, a bead, or a chromatographic resin, as well as non-covalent, high affinity interactions such as antibody binding, or use of streptavidin/biotin binding wherein the immobilized compound includes a biotin moiety. Detection of binding can be accomplished (i) using a radioactive label on the compound that is not immobilized, (ii) using of a fluorescent label on the non-immobilized compound, (iii) using an antibody immunospecific for the non-immobilized compound, (iv) using a label on the non-immobilized compound that excites a fluorescent support to which the immobilized compound is attached, as well as other techniques well known and routinely practiced in the art.

The invention also provides cell-based assays to identify binding partner compounds of an ion-x polypeptide. In one embodiment, the invention provides a method comprising the steps of contacting an ion-x polypeptide expressed on the surface of a cell with a candidate binding partner compound and detecting binding of the candidate binding partner compound to the ion-x polypeptide. In a preferred embodiment, the detection comprises detecting a calcium flux or other physiological event in the cell caused by the binding of the molecule.

Another aspect of the present invention is directed to methods of identifying compounds that bind to either ion-x or nucleic acid molecules encoding ion-x, comprising contacting ion-x, or a nucleic acid molecule encoding the same, with a compound, and determining whether the compound binds ion-x or a nucleic acid molecule encoding the same. Binding can be determined by binding assays which are well known to the skilled artisan, including, but not limited to, gel-shift assays, Western blots, radiolabeled competition assay, phage-based expression cloning, co-fractionation by chromatography, co-precipitation, cross linking, interaction trap/two-hybrid analysis, southwestern analysis, ELISA, and the like, which are described in, for example, Current Protocols in Molecular Biology, 1999, John Wiley & Sons, NY, which is incorporated herein by reference in its entirety. The compounds to be screened include (which may include compounds which are suspected to bind ion-x, or a nucleic acid molecule encoding the same), but are not limited to, extracellular, intracellular, biologic or chemical origin. The methods of the invention also embrace ligands, especially neuropeptides, that are attached to a label, such as a radiolabel (e.g., ¹²⁵I, ³⁵S, ³²P, ³³P, ³H), a fluorescence label, a chemiluminescent label, an enzymic label and an immunogenic label. Modulators falling within the scope of the invention include, but are not limited to, non-peptide molecules such as non-peptide mimetics, non-peptide allosteric effectors, and peptides. The ion-x polypeptide or polynucleotide employed in such a test may either be free in solution, attached to a solid support, borne on a cell surface or located intracellularly or associated with a portion of a cell. One skilled in the art can, for example, measure the formation of complexes between ion-x and the compound being tested. Alternatively, one skilled in the art can examine the diminution in complex formation between ion-x and its substrate caused by the compound being tested.

In another embodiment of the invention, high throughput screening for compounds having suitable binding affinity to ion-x is employed. Briefly, large numbers of different small peptide test compounds are synthesized on a solid substrate. The peptide test compounds are contacted with ion-x and washed. Bound ion-x is then detected by methods well known in the art. Purified polypeptides of the invention can also be coated directly onto plates for use in the aforementioned drug screening techniques. In addition, non-neutralizing antibodies can be used to capture the protein and immobilize it on the solid support.

Generally, an expressed ion-x can be used for HTS binding assays in conjunction with its defined ligand, in this case the corresponding neuropeptide that activates it. The identified peptide is labeled with a suitable radioisotope, including, but not limited to, ¹²⁵I, ³H, ³⁵S or ³²P, by methods that are well known to those skilled in the art. Alternatively, the peptides may be labeled by well-known methods with a suitable fluorescent derivative (Baindur et al., Drug Dev. Res., 1994, 33, 373-398; Rogers, Drug Discovery Today, 1997, 2, 156-160). Radioactive ligand specifically bound to the receptor in membrane preparations made from the cell line expressing the recombinant protein can be detected in HTS assays in one of several standard ways, including filtration of the receptor-ligand complex to separate bound ligand from unbound ligand (Williams, Med. Res. Rev., 1991, 11, 147-184; Sweetnam et al., J. Natural Products, 1993, 56, 441-455). Alternative methods include a scintillation proximity assay (SPA) or a FlashPlate format in which such separation is unnecessary (Nakayama, Cur. Opinion Drug Disc. Dev., 1998, 1, 85-91 Bossé et al., J. Biomolecular Screening, 1998, 3, 285-292.). Binding of fluorescent ligands can be detected in various ways, including fluorescence energy transfer (FRET), direct spectrophotofluorometric analysis of bound ligand, or fluorescence polarization (Rogers, Drug Discovery Today, 1997, 2, 156-160; Hill, Cur. Opinion Drug Disc. Dev., 1998, 1, 92-97).

Other assays may be used to identify specific ligands of a ion-x receptor, including assays that identify ligands of the target protein through measuring direct binding of test ligands to the target protein, as well as assays that identify ligands of target proteins through affinity ultrafiltration with ion spray mass spectroscopy/HPLC methods or other physical and analytical methods. Alternatively, such binding interactions are evaluated indirectly using the yeast two-hybrid system described in Fields et al., Nature, 340:245-246 (1989), and Fields et al., Trends in Genetics, 10:286-292 (1994), both of which are incorporated herein by reference. The two-hybrid system is a genetic assay for detecting interactions between two proteins or polypeptides. It can be used to identify proteins that bind to a known protein of interest, or to delineate domains or residues critical for an interaction. Variations on this methodology have been developed to clone genes that encode DNA binding proteins, to identify peptides that bind to a protein, and to screen for drugs. The two-hybrid system exploits the ability of a pair of interacting proteins to bring a transcription activation domain into close proximity with a DNA binding domain that binds to an upstream activation sequence (UAS) of a reporter gene, and is generally performed in yeast. The assay requires the construction of two hybrid genes encoding (1) a DNA-binding domain that is fused to a first protein and (2) an activation domain fused to a second protein. The DNA-binding domain targets the first hybrid protein to the UAS of the reporter gene; however, because most proteins lack an activation domain, this DNA-binding hybrid protein does not activate transcription of the reporter gene. The second hybrid protein, which contains the activation domain, cannot by itself activate expression of the reporter gene because it does not bind the UAS. However, when both hybrid proteins are present, the noncovalent interaction of the first and second proteins tethers the activation domain to the UAS, activating transcription of the reporter gene. For example, when the first protein is an ion channel gene product, or fragment thereof, that is known to interact with another protein or nucleic acid, this assay can be used to detect agents that interfere with the binding interaction. Expression of the reporter gene is monitored as different test agents are added to the system. The presence of an inhibitory agent results in lack of a reporter signal.

The yeast two-hybrid assay can also be used to identify proteins that bind to the gene product. In an assay to identify proteins that bind to an ion-x receptor, or fragment thereof, a fusion polynucleotide encoding both an ion-x receptor (or fragment) and a UAS binding domain (i.e., a first protein) may be used. In addition, a large number of hybrid genes each encoding a different second protein fused to an activation domain are produced and screened in the assay. Typically, the second protein is encoded by one or more members of a total cDNA or genomic DNA fusion library, with each second protein-coding region being fused to the activation domain. This system is applicable to a wide variety of proteins, and it is not even necessary to know the identity or function of the second binding protein. The system is highly sensitive and can detect interactions not revealed by other methods; even transient interactions may trigger transcription to produce a stable MRNA that can be repeatedly translated to yield the reporter protein.

Other assays may be used to search for agents that bind to the target protein. One such screening method to identify direct binding of test ligands to a target protein is described in U.S. Pat. No. 5,585,277, incorporated herein by reference. This method relies on the principle that proteins generally exist as a mixture of folded and unfolded states, and continually alternate between the two states. When a test ligand binds to the folded form of a target protein (i.e., when the test ligand is a ligand of the target protein), the target protein molecule bound by the ligand remains in its folded state. Thus, the folded target protein is present to a greater extent in the presence of a test ligand which binds the target protein, than in the absence of a ligand. Binding of the ligand to the target protein can be determined by any method that distinguishes between the folded and unfolded states of the target protein. The function of the target protein need not be known in order for this assay to be performed. Virtually any agent can be assessed by this method as a test ligand, including, but not limited to, metals, polypeptides, proteins, lipids, polysaccharides, polynucleotides and small organic molecules.

Another method for identifying ligands of a target protein is described in Wieboldt et al., Anal. Chem., 69:1683-1691 (1997), incorporated herein by reference. This technique screens combinatorial libraries of 20-30 agents at a time in solution phase for binding to the target protein. Agents that bind to the target protein are separated from other library components by simple membrane washing. The specifically selected molecules that are retained on the filter are subsequently liberated from the target protein and analyzed by HPLC and pneumatically assisted electrospray (ion spray) ionization mass spectroscopy. This procedure selects library components with the greatest affinity for the target protein, and is particularly useful for small molecule libraries.

Other embodiments of the invention comprise using competitive screening assays in which neutralizing antibodies capable of binding a polypeptide of the invention specifically compete with a test compound for binding to the polypeptide. In this manner, the antibodies can be used to detect the presence of any peptide that shares one or more antigenic determinants with ion-x. Radiolabeled competitive binding studies are described in A. H. Lin et al. Antimicrobial Agents and Chemotherapy, 1997, vol. 41, no. 10. pp. 2127-2131, the disclosure of which is incorporated herein by reference in its entirety.

Identification of Modulating Agents

The invention also provides methods for identifying a modulator of binding between a ion-x and an ion-x binding partner, comprising the steps of: (a) contacting an ion-x binding partner and a composition comprising an ion-x in the presence and in the absence of a putative modulator compound; (b) detecting binding between the binding partner and the ion-x; and (c) identifying a putative modulator compound or a modulator compound in view of decreased or increased binding between the binding partner and the ion-x in the presence of the putative modulator, as compared to binding in the absence of the putative modulator. Compounds identified as modulating binding between ion-x and an ion-x binding partner may be further tested in other assays including, but not limited to, in vivo models, in order to confirm or quantitate their activity.

Ion-x binding partners that stimulate ion-x activity are useful as agonists in disease states or conditions characterized by insufficient ion-x signaling (e.g., as a result of insufficient activity of an ion-x ligand). Ion-x binding partners that block ligand-mediated ion-x signaling are useful as ion-x antagonists to treat disease states or conditions characterized by excessive ion-x signaling. In addition ion-x modulators in general, as well as ion-x polynucleotides and polypeptides, are useful in diagnostic assays for such diseases or conditions.

In another aspect, the invention provides methods for treating a disease or abnormal condition by administering to a patient in need of such treatment a substance that modulates the activity or expression of a polypeptide having a sequence selected from the group consisting of SEQ ID NO:52 to SEQ ID NO:102, and SEQ ID NOS: 105, 106, 109, and 110.

Agents that modulate (i.e., increase, decrease, or block) ion-x activity or expression may be identified by incubating a putative modulator with a cell containing an ion-x polypeptide or polynucleotide and determining the effect of the putative modulator on ion-x activity or expression. The selectivity of a compound that modulates the activity of ion-x can be evaluated by comparing its effects on ion-x to its effect on other ion channel compounds. Selective modulators may include, for example, antibodies and other proteins, peptides, or organic molecules that specifically bind to an ion-x polypeptide or an ion-x -encoding nucleic acid. Modulators of ion-x activity will be therapeutically useful in treatment of diseases and physiological conditions in which normal or aberrant ion-x activity is involved. Compounds identified as modulating ion-x activity may be further tested in other assays including, but not limited to, in vivo models, in order to confirm or quantitate their activity.

Ion-x polynucleotides, polypeptides, and modulators may be used in the treatment of such diseases and conditions as infections, such as viral infections caused by HIV-1 or HIV-2; thyroid disorders (e.g. thyreotoxicosis, myxoedema); renal failure; inflammatory conditions (e.g., Crohn's disease); diseases related to cell differentiation and homeostasis; rheumatoid arthritis; autoimmune disorders; movement disorders; CNS disorders (e.g., pain including neuropathic pain, migraine, and other headaches; stroke; psychotic and neurological disorders, including anxiety, schizophrenia, manic depression, anxiety, generalized anxiety disorder, post-traumatic-stress disorder, depression, bipolar disorder, delirium, dementia, severe mental retardation; dyskinesias, such as Huntington's disease or Tourette's Syndrome; attention disorders including ADD and ADHD, and degenerative disorders such as Parkinson's, Alzheimer's; movement disorders, including ataxias, supranuclear palsy, etc.); infections, such as viral infections caused by HIV-1 or HIV-2; metabolic and cardiovascular diseases and disorders (e.g., type 2 diabetes, obesity, anorexia, hypotension, hypertension, thrombosis, myocardial infarction, cardiomyopathies, atherosclerosis, etc.); proliferative diseases and cancers (e.g., different cancers such as breast, colon, lung, etc., and hyperproliferative disorders such as psoriasis, prostate hyperplasia, etc.); hormonal disorders (e.g., male/female hormonal replacement, polycystic ovarian syndrome, alopecia, etc.); and sexual dysfunction, among others. Ion-x polynucleotides and polypeptides, as well as ion-x modulators, may also be used in diagnostic assays for such diseases or conditions.

Methods of the invention to identify modulators include variations on any of the methods described above to identify binding partner compounds, the variations including techniques wherein a binding partner compound has been identified and the binding assay is carried out in the presence and absence of a candidate modulator. A modulator is identified in those instances where binding between the ion-x polypeptide and the binding partner compound changes in the presence of the candidate modulator compared to binding in the absence of the candidate modulator compound. A modulator that increases binding between the ion-x polypeptide and the binding partner compound is described as an enhancer or activator, and a modulator that decreases binding between the ion-x polypeptide and the binding partner compound is described as an inhibitor.

The invention also comprehends high-throughput screening (HTS) assays to identify compounds that interact with or inhibit biological activity (i.e., affect enzymatic activity, binding activity, etc.) of an ion-x polypeptide. HTS assays permit screening of large numbers of compounds in an efficient manner. Cell-based HTS systems are contemplated to investigate ion-x receptor-ligand interaction. HTS assays are designed to identify “hits” or “lead compounds” having the desired property, from which modifications can be designed to improve the desired property. Chemical modification of the “hit” or “lead compound” is often based on an identifiable structure/activity relationship between the “hit” and the ion-x polypeptide.

Another aspect of the present invention is directed to methods of identifying compounds which modulate (i.e., increase or decrease) activity of ion-x comprising contacting ion-x with a compound, and determining whether the compound modifies activity of ion-x. The activity in the presence of the test compared is measured to the activity in the absence of the test compound. One of skill in the art can, for example, measure the activity of the ion channel polypeptide using electrophysiological methods, described infra. Where the activity of the sample containing the test compound is higher than the activity in the sample lacking the test compound, the compound will have increased activity. Similarly, where the activity of the sample containing the test compound is lower than the activity in the sample lacking the test compound, the compound will have inhibited activity.

The activity of the polypeptides of the invention can also be determined by, as non-limiting examples, the ability to bind or be activated by certain ligands, including, but not limited to, known neurotransmitters, agonists and antagonists, including but not limited to serotonin, acetylcholine, nicotine, and GABA. Alternatively, the activity of the ion channels can be assayed by examining activity such as ability to bind or be affected by calcium ions, hormones, chemokines, neuropeptides, neurotransmitters, nucleotides, lipids, odorants, and photons. In various embodiments of the method, the assay may take the form of an ion flux assay, a membrane potential assay, a yeast growth assay, a cAMP assay, an inositol triphosphate assay, a diacylglycerol assay, an Aequorin assay, a Luciferase assay, a FLIPR assay for intracellular Ca²⁺ concentration, a mitogenesis assay, a MAP Kinase activity assay, an arachidonic acid release assay (e.g., using [³H]-arachidonic acid), and an assay for extracellular acidification rates, as well as other binding or function-based assays of activity that are generally known in the art Another potentially useful assay to examine the activity of ion channels is electrophysiology, the measurement of ion permeability across the cell membrane. This technique is described in, for example, Electrophysiology, A Practical Approach, DI Wallis editor, IRL Press at Oxford University Press, (1993), and Voltage and patch Clamping with Microelectrodes, Smith et al., eds., Waverly Press, Inc for the American Physiology Society (1985), each of which is incorporated by reference in its entirety.

Another assay to examine the activity of ion channels is through the use of the FLIPR Fluorometric Imaging Plate Reader system, developed by Dr. Vince Groppi of the Pharmacia Corporation to perform cell-based, high-throughput screening (HTS) assays measuring, for example, membrane potential. Changes in plasma membrane potential correlate with the modulation of ion channels as ions move into or out of the cell. The FLIPR system measures such changes in membrane potential. This is accomplished by loading cells expressing an ion channel gene with a cell-membrane permeant fluorescent indicator dye suitable for measuring changes in membrane potential such as diBAC (bis-(1,3-dibutylbarbituric acid) pentamethine oxonol, Molecular Probes). Thus the modulation of ion channel activity can be assessed with FLIPR and detected as changes in the emission spectrum of the diBAC dye.

The present invention is particularly useful for screening compounds by using ion-x in any of a variety of drug screening techniques. The compounds to be screened include (which may include compounds which are suspected to modulate ion-x activity), but are not limited to, extracellular, intracellular, biologic or chemical origin. The ion-x polypeptide employed in such a test may be in any form, preferably, free in solution, attached to a solid support, borne on a cell surface or located intracellularly. One skilled in the art can, for example, measure the formation of complexes between ion-x and the compound being tested. Alternatively, one skilled in the art can examine the diminution in complex formation between ion-x and its substrate caused by the compound being tested.

The activity of ion-x polypeptides of the invention can be determined by, for example, examining the ability to bind or be activated by chemically synthesized peptide ligands. Alternatively, the activity of ion-x polypeptides can be assayed by examining their ability to bind calcium ions, hormones, chemokines, neuropeptides, neurotransmitters, nucleotides, lipids, odorants, and photons. Alternatively, the activity of the ion-x polypeptides can be determined by examining the activity of effector molecules including, but not limited to, adenylate cyclase, phospholipases and ion channels. Thus, modulators of ion-x polypeptide activity may alter ion channel function, such as a binding property of a channel or an activity such as ion selectivity. In various embodiments of the method, the assay may take the form of an ion flux assay, a yeast growth assay, a cAMP assay, an inositol triphosphate assay, a diacylglycerol assay, an Aequorin assay, a Luciferase assay, a FLIPR assay for intracellular Ca²+ concentration, a mitogenesis assay, a MAP Kinase activity assay, an arachidonic acid release assay (e.g., using [³H]-arachidonic acid), and an assay for extracellular acidification rates, as well as other binding or function-based assays of ion-x activity that are generally known in the art. Ion-x activity can be determined by methodologies that are used to assay for FaRP activity, which is well known to those skilled in the art. Biological activities of ion-x receptors according to the invention include, but are not limited to, the binding of a natural or an unnatural ligand, as well as any one of the functional activities of ion channels known in the art.

The modulators of the invention exhibit a variety of chemical structures, which can be generally grouped into non-peptide mimetics of natural ion channel ligands, peptide and non-peptide allosteric effectors of ion channels, and peptides that may function as activators or inhibitors (competitive, uncompetitive and non-competitive) (e.g., antibody products) of ion channels. The invention does not restrict the sources for suitable modulators, which may be obtained from natural sources such as plant, animal or mineral extracts, or non-natural sources such as small molecule libraries, including the products of combinatorial chemical approaches to library construction, and peptide libraries.

Examples of organic modulators of ion channels are GABA, serotonin, acetylcholine, nicotine, glutamate, glycine, NMDA, and kainic acid.

Other assays can be used to examine enzymatic activity including, but not limited to, photometric, radiometric, HPLC, electrochemical, and the like, which are described in, for example, Enzyme Assays: A Practical Approach, eds., R. Eisenthal and M. J. Danson, 1992, Oxford University Press, which is incorporated herein by reference in its entirety.

The use of cDNAs encoding ion channels in drug discovery programs is well known; assays capable of testing thousands of unknown compounds per day in high-throughput screens (HTSs) are thoroughly documented. The literature is replete with examples of the use of radiolabeled ligands in HTS binding assays for drug discovery (see Williams, Medicinal Research Reviews, 1991, 11, 147-184.; Sweetnam, et al., J. Natural Products, 1993, 56, 441-455 for review). Recombinant receptors are preferred for binding assay HTS because they allow for better specificity (higher relative purity), provide the ability to generate large amounts of receptor material, and can be used in a broad variety of formats (see Hodgson, Bio/Technology, 1992, 10, 973-980; each of which is incorporated herein by reference in its entirety).

A variety of heterologous systems are available for functional expression of recombinant receptors that are well known to those skilled in the art. Such systems include bacteria (Strosberg, et al., Trends in Pharmacological Sciences, 1992, 13, 95-98), yeast (Pausch, Trends in Biotechnology, 1997, 15, 487-494), several kinds of insect cells (Vanden Broeck, Int. Rev. Cytology, 1996, 164, 189-268), amphibian cells (Jayawickreme et al., Current Opinion in Biotechnology, 1997, 8, 629-634) and several mammalian cell lines (CHO, HEK-293, COS, etc.; see Gerhardt, et al., Eur. J. Pharmacology, 1997, 334, 1-23). These examples do not preclude the use of other possible cell expression systems, including cell lines obtained from nematodes (PCT application WO 98/37177).

In preferred embodiments of the invention, methods of screening for compounds that modulate ion-x activity comprise contacting test compounds with ion-x and assaying for the presence of a complex between the compound and ion-x. In such assays, the ligand is typically labeled. After suitable incubation, free ligand is separated from that present in bound form, and the amount of free or uncomplexed label is a measure of the ability of the particular compound to bind to ion-x.

Examples of such biological responses include, but are not limited to, the following: the ability to survive in the absence of a limiting nutrient in specifically engineered yeast cells (Pausch, Trends in Biotechnology, 1997, 15, 487-494); changes in intracellular Ca²⁺ concentration as measured by fluorescent dyes (Murphy, et al., Cur. Opinion Drug Disc. Dev., 1998, 1, 192-199). Fluorescence changes can also be used to monitor ligand-induced changes in membrane potential or intracellular pH; an automated system suitable for HTS has been described for these purposes (Schroeder, et al., J. Biomolecular Screening, 1996, 1, 75-80). Melanophores prepared from Xenopus laevis show a ligand-dependent change in pigment organization in response to heterologous ion channel activation; this response is adaptable to HTS formats (Jayawickreme et al., Cur. Opinion Biotechnology, 1997, 8, 629-634). Assays are also available for the measurement of common second messengers, including cAMP, phosphoinositides and arachidonic acid, but these are not generally preferred for HTS.

In another embodiment of the invention, permanently transfected CHO cells could b e used for the preparation of membranes which contain significant amounts of the recombinant receptor proteins; these membrane preparations would then be used in receptor binding assays, employing the radiolabeled ligand specific for the particular receptor. Altern atively, a functional assay, such as fluorescent monitoring of ligand-induced changes in internal Ca²⁺ concentration or membrane potential in permanently transfected CHO cells containing each of these receptors individually or in combination would be preferred for HTS. Equally preferred would be an alternative type of mammalian cell, such as HEK-293 or COS cells, in similar formats. More preferred would be permanently transfected insect cell lines, such as Drosophila S2 cells. Even more preferred would be recombinant yeast cells expressing the Drosophila melanogaster receptors in HTS formats well known to those skilled in the art (e.g., Pausch, Trends in Biotechnology, 1997, 15, 487-494).

The invention contemplates a multitude of assays to screen and identify inhibitors of ligand binding to ion-x. In one example, the ion-x is immobilized and interaction with a binding partner is assessed in the presence and absence of a candidate modulator such as an inhibitor compound. In another example, interaction between the ion-x and its binding partner is assessed in a solution assay, both in the presence and absence of a candidate inhibitor compound. In either assay, an inhibitor is identified as a compound that decreases binding between the ion-x and its binding partner. Another contemplated assay involves a variation of the dihybrid assay wherein an inhibitor of protein/protein interactions is identified by detection of a positive signal in a transformed or transfected host cell, as described in PCT publication number WO 95/20652, published Aug. 3, 1995.

Candidate modulators contemplated by the invention include compounds selected from libraries of either potential activators or potential inhibitors. There are a number of different libraries used for the identification of small molecule modulators, including: (1) chemical libraries, (2) natural product libraries, and (3) combinatorial libraries comprised of random peptides, oligonucleotides or organic molecules. Chemical libraries consist of random chemical structures, some of which are analogs of known compounds or analogs of compounds that have been identified as “hits” or “leads” in other drug discovery screens, some of which are derived from natural products, and some of which arise from non-directed synthetic organic chemistry. Natural product libraries are collections of microorganisms, animals, plants, or marine organisms that are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. For a review, see Science 282:63-68 (1998). Combinatorial libraries are composed of large numbers of peptides, oligonucleotides, or organic compounds as a mixture. These libraries are relatively easy to prepare by traditional automated synthesis methods, PCR, cloning, or proprietary synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997). Identification of modulators through use of the various libraries described herein permits modification of the candidate “hit” (or “lead”) to optimize the capacity of the “hit” to modulate activity.

Still other candidate inhibitors contemplated by the invention can be designed and include soluble forms of binding partners, as well as such binding partners as chimeric, or fusion, proteins. A “binding partner” as used herein broadly encompasses non-peptide modulators, as well as such peptide modulators as neuropeptides other than natural ligands, antibodies, antibody fragments, and modified compounds comprising antibody domains that are immunospecific for the expression product of the identified ion-x gene.

The polypeptides of the invention are employed as a research tool for identification, characterization and purification of interacting, regulatory proteins. Appropriate labels are incorporated into the polypeptides of the invention by various methods known in the art and the polypeptides are used to capture interacting molecules. For example, molecules are incubated with the labeled polypeptides, washed to remove unbound polypeptides, and the polypeptide complex is quantified. Data obtained using different concentrations of polypeptide are used to calculate values for the number, affinity, and association of polypeptide with the protein complex.

Labeled polypeptides are also useful as reagents for the purification of molecules with which the polypeptide interacts including, but not limited to, inhibitors. In one embodiment of affinity purification, a polypeptide is covalently coupled to a chromatography column. Cells and their membranes are extracted, and various cellular subcomponents are passed over the column. Molecules bind to the column by virtue of their affinity to the polypeptide. The polypeptide-complex is recovered from the column, dissociated and the recovered molecule is subjected to protein sequencing. This amino acid sequence is then used to identify the captured molecule or to design degenerate oligonucleotides for cloning the corresponding gene from an appropriate cDNA library.

Alternatively, compounds may be identified which exhibit similar properties to the ligand for the ion-x of the invention, but which are smaller and exhibit a longer half time than the endogenous ligand in a human or animal body. When an organic compound is designed, a molecule according to the invention is used as a “lead” compound. The design of mimetics to known pharmaceutically active compounds is a well-known approach in the development of pharmaceuticals based on such “lead” compounds. Mimetic design, synthesis and testing are generally used to avoid randomly screening a large number of molecules for a target property. Furthermore, structural data deriving from the analysis of the deduced amino acid sequences encoded by the DNAs of the present invention are useful to design new drugs, more specific and therefore with a higher pharmacological potency.

Comparison of the protein sequences of the present invention with the sequences present in all the available databases showed a significant homology with the transmembrane domains, including the pore domain, of ion channel proteins. Accordingly, computer modeling can be used to develop a putative tertiary structure of the proteins of the invention based on the available information of the transmembrane domain of other proteins. Thus, novel ligands based on the predicted structure of ion-x can be designed.

In a particular embodiment, the novel molecules identified by the screening methods according to the invention are low molecular weight organic molecules, in which case a composition or pharmaceutical composition can be prepared thereof for oral intake, such as in tablets. The compositions, or pharmaceutical compositions, comprising the nucleic acid molecules, vectors, polypeptides, antibodies and compounds identified by the screening methods described herein, can be prepared for any route of administration including, but not limited to, oral, intravenous, cutaneous, subcutaneous, nasal, intramuscular or intraperitoneal. The nature of the carrier or other ingredients will depend on the specific route of administration and particular embodiment of the invention to be administered. Examples of techniques and protocols that are useful in this context are, inter alia, found in Remington's Pharmaceutical Sciences, 16^{th} edition, Osol, A (ed.), 1980, which is incorporated herein by reference in its entirety.

The dosage of these low molecular weight compounds will depend on the disease state or condition to be treated and other clinical factors such as weight and condition of the human or animal and the route of administration of the compound. For treating human or animals, between approximately 0.5 mg/kg of body weight to 500 mg/kg of body weight of the compound can be administered. Therapy is typically administered at lower dosages and is continued until the desired therapeutic outcome is observed.

The present compounds and methods, including nucleic acid molecules, polypeptides, antibodies, compounds identified by the screening methods described herein, have a variety of pharmaceutical applications and may be used, for example, to treat or prevent unregulated cellular growth, such as cancer cell and tumor growth. In a particular embodiment, the present molecules are used in gene therapy. For a review of gene therapy procedures, see e.g. Anderson, Science, 1992, 256, 808-813, which is incorporated herein by reference in its entirety.

The present invention also encompasses a method of agonizing (stimulating) or antagonizing an ion-x natural binding partner associated activity in a mammal comprising administering to said mammal an agonist or antagonist to one of the above disclosed polypeptides in an amount sufficient to effect said agonism or antagonism. One embodiment of the present invention, then, is a method of treating diseases in a mammal with an agonist or antagonist of the protein of the present invention comprises administering the agonist or antagonist to a mammal in an amount sufficient to agonize or antagonize ion-x-associated functions.

Exemplary diseases and conditions amenable to treatment based on the present invention include, but are not limited to, thyroid disorders (e.g. thyreotoxicosis, myxoedema); renal failure; inflammatory conditions (e.g., Crohn's disease); diseases related to cell differentiation and homeostasis; rheumatoid arthritis; autoimmune disorders; movement disorders; CNS disorders (e.g., pain including neuropathic pain, migraine, and other headaches; stroke; psychotic and neurological disorders, including anxiety, schizophrenia, manic depression, anxiety, generalized anxiety disorder, post-traumatic-stress disorder, depression, bipolar disorder, delirium, dementia, severe mental retardation; dyskinesias, such as Huntington's disease or Tourette's Syndrome; attention disorders including ADD and ADHD, and degenerative disorders such as Parkinson's, Alzheimer's; movement disorders, including ataxias, supranuclear palsy, etc.); infections, such as viral infections caused by HIV-1 or HIV-2; metabolic and cardiovascular diseases and disorders (e.g., type 2 diabetes, obesity, anorexia, hypotension, hypertension, thrombosis, myocardial infarction, cardiomyopathies, atherosclerosis, etc.); proliferative diseases and cancers (e.g., different cancers such as breast, colon, lung, etc., and hyperproliferative disorders such as psoriasis, prostate hyperplasia, etc.); hormonal disorders (e.g., male/female hormonal replacement, polycystic ovarian syndrome, alopecia, etc.); and sexual dysfunction, among others.

Compounds that can traverse cell membranes and are resistant to acid hydrolysis are potentially advantageous as therapeutics as they can become highly bioavailable after being administered orally to patients. However, many of these protein inhibitors only weakly inhibit function. In addition, many inhibit a variety of protein kinases and will therefore cause multiple side effects as therapeutics for diseases.

Methods of determining the dosages of compounds to be administered to a patient and modes of administering compounds to an organism are disclosed in U.S. application Ser. No. 08/702,282, filed Aug. 23, 1996 and International patent publication number WO 96/22976, published Aug. 1, 1996, both of which are incorporated herein by reference in their entirety, including any drawings, figures or tables. Those skilled in the art will appreciate that such descriptions are applicable to the present invention and can be easily adapted to it.

The proper dosage depends on various factors such as the type of disease being treated, the particular composition being used and the size and physiological condition of the patient. Therapeutically effective doses for the compounds described herein can be estimated initially from cell culture and animal models. For example, a dose can be formulated in animal models to achieve a circulating concentration range that initially takes into account the IC₅₀ as determined in cell culture assays. The animal model data can be used to more accurately determine useful doses in humans.

Plasma half-life and biodistribution of the drug and metabolites in the plasma, tumors and major organs can also be determined to facilitate the selection of drugs most appropriate to inhibit a disorder. Such measurements can be carried out. For example, HPLC analysis can be performed on the plasma of animals treated with the drug and the location of radiolabeled compounds can be determined using detection methods such as X-ray, CAT scan and MRI. Compounds that show potent inhibitory activity in the screening assays, but have poor pharmacokinetic characteristics, can be optimized by altering the chemical structure and retesting. In this regard, compounds displaying good pharmacokinetic characteristics can be used as a model.

Toxicity studies can also be carried out by measuring the blood cell composition. For example, toxicity studies can be carried out in a suitable animal model as follows: 1) the compound is administered to mice (an untreated control mouse should also be used); 2) blood samples are periodically obtained via the tail vein from one mouse in each treatment group; and 3) the samples are analyzed for red and white blood cell counts, blood cell composition and the percent of lymphocytes versus polymorphonuclear cells. A comparison of results for each dosing regime with the controls indicates if toxicity is present.

At the termination of each toxicity study, further studies can be carried out by sacrificing the animals (preferably, in accordance with the American Veterinary Medical Association guidelines Report of the American Veterinary Medical Assoc. Panel on Euthanasia, Journal of American Veterinary Medical Assoc., 202:229-249, 1993). Representative animals from each treatment group can then be examined by gross necropsy for immediate evidence of metastasis, unusual illness or toxicity. Gross abnormalities in tissue are noted and tissues are examined histologically. Compounds causing a reduction in body weight or blood components are less preferred, as are compounds having an adverse effect on major organs. In general, the greater the adverse effect the less preferred the compound.

For the treatment of cancers the expected daily dose of a hydrophobic pharmaceutical agent is between 1 to 500 mg/day, preferably 1 to 250 mg/day, and most preferably 1 to 50 mg/day. Drugs can be delivered less frequently provided plasma levels of the active moiety are sufficient to maintain therapeutic effectiveness. Plasma levels should reflect the potency of the drug. Generally, the more potent the compound the lower the plasma levels necessary to achieve efficacy.

Ion-x mRNA transcripts may found in many tissues, including, but not limited to, brain, kidney, colon, small intestine, stomach, testis, placenta, adrenal gland, peripheral blood leukocytes, bone marrow, retina, ovary, fetal brain, fetal liver, heart, spleen, liver, kidney, lung, muscle, thyroid gland, uterus, prostate, skin, salivary gland, and pancreas. Tissues where specific ion-x mRNA transcripts are expressed are identified in the Examples, below.

Sequences selected from the group consisting of SEQ ID NO:1 to SEQ ID NO:51, and SEQ ID NOS:103, 104, 107, and 108, and fragments thereof, will, as detailed above, enable screening the endogenous neurotransmitters/hormones/ligands which activate, agonize, or antagonize ion-x and for compounds with potential utility in treating disorders including, but not limited to, thyroid disorders (e.g. thyreotoxicosis, myxoedema); renal failure; inflammatory conditions (e.g., Crohn's disease); diseases related to cell differentiation and homeostasis; rheumatoid arthritis; autoimmune disorders; movement disorders; CNS disorders (e.g., pain including neuropathic pain, migraine, and other headaches; stroke; psychotic and neurological disorders, including anxiety, schizophrenia, manic depression, anxiety, generalized anxiety disorder, post-traumatic-stress disorder, depression, bipolar disorder, delirium, dementia, severe mental retardation; dyskinesias, such as Huntington's disease or Tourette's Syndrome; attention disorders including ADD and ADHD, and degenerative disorders such as Parkinson's, Alzheimer's; movement disorders, including ataxias, supranuclear palsy, etc.); infections, such as viral infections caused by HIV-1 or HIV-2; metabolic and cardiovascular diseases and disorders (e.g., type 2 diabetes, obesity, anorexia, hypotension, hypertension, thrombosis, myocardial infarction, cardiomyopathies, atherosclerosis, etc.); proliferative diseases and cancers (e.g., different cancers such as breast, colon, lung, etc., and hyperproliferative disorders such as psoriasis, prostate hyperplasia, etc.); hormonal disorders (e.g., male/female hormonal replacement, polycystic ovarian syndrome, alopecia, etc.); and sexual dysfunction, among others.

For example, ion-x may be useful in the treatment of respiratory ailments such as asthma, where T cells are implicated by the disease. Contraction of airway smooth muscle is stimulated by thrombin. Cicala et al (1999) Br J Pharmacol 126:478-484. Additionally, in bronchiolitis obliterans, it has been noted that activation of thrombin receptors may be deleterious. Hauck et al.(1999) Am J Physiol 277:L22-L29. Furthermore, mast cells have also been shown to have thrombin receptors. Cirino et al (1996) J Exp Med 183:821-827. Ion-x may also be useful in remodeling of airway structures in chronic pulmonary inflammation via stimulation of fibroblast procollagen synthesis. See, e.g., Chambers et al. (1998) Biochem J 333:121-127; Trejo et al. (1996) J Biol Chem 271:21536-21541.

In another example, increased release of sCD40L and expression of CD40L by T cells after activation of thrombin receptors suggests that ion-x may be useful in the treatment of unstable angina due to the role of T cells and inflammation. See Aukrust et al. (1999) Circulation 100:614-620.

A further example is the treatment of inflammatory diseases, such as psoriasis, inflammatory bowel disease, multiple sclerosis, rheumatoid arthritis, and thyroiditis. Due to the tissue expression profile of ion-x, inhibition of thrombin receptors may be beneficial for these diseases. See, e.g., Morris et al. (1996) Ann Rheum Dis 55:841-843. In addition to T cells, NK cells and monocytes are also critical cell types which contribute to the pathogenesis of these diseases. See, e.g., Naldini & Carney (1996) Cell Immunol 172:35-42; Hoffman & Cooper (1995) Blood Cells Mol Dis 21:156-167; Colotta et al. (1994) Am J Pathol 144:975-985.

Expression of ion-x in spleen may suggest that it may play a role in the proliferation of hematopoietic progenitor cells. See DiCuccio et al. (1996) Exp Hematol 24:914-918.

As another example, ion-x may be useful in the treatment of acute and/or traumatic brain injury. Astrocytes have been demonstrated to express thrombin receptors. Activation of thrombin receptors may be involved in astrogliosis following brain injury. Therefore, inhibition of receptor activity may be beneficial for limiting neuroinflammation. Scar formation mediated by astrocytes may also be limited by inhibiting thrombin receptors. See, e.g, Pindon et al. (1998) Eur J Biochem 255:766-774; Ubl & Reiser. (1997) Glia 21:361-369; Grabham & Cunningham (1995) J Neurochem 64:583-591.

Ion-x receptor activation may mediate neuronal and astrocyte apoptosis and prevention of neurite outgrowth. Inhibition would be beneficial in both chronic and acute brain injury. See, e.g., Donovan et al. (1997) J Neurosci 17:5316-5326; Turgeon et al (1998) J Neurosci 18:6882-6891; Smith-Swintosky et al. (1997) J Neurochem 69:1890-1896; Gill et al. (1998) Brain Res 797:321-327; Suidan et al. (1996) Semin Thromb Hemost 22:125-133.

The attached Sequence Listing contains the sequences of the polynucleotides and polypeptides of the invention and is incorporated herein by reference in its entirety.

The identification of modulators such as agonists and antagonists is therefore useful for the identification of compounds useful to treat neurological diseases and disorders. Such neurological diseases and disorders, include, but are not limited to, schizophrenia, affective disorders, ADHD/ADD (i.e., Attention Deficit-Hyperactivity Disorder/Attention Deficit Disorder), and neural disorders such as Alzheimer's disease, Parkinson's disease, migraine, and senile dementia as well as depression, anxiety, bipolar disease, epilepsy, neuritis, neurasthenia, neuropathy, neuroses, and the like.

Methods of Screening Human Subjects

Thus in yet another embodiment, the invention provides genetic screening procedures that entail analyzing a person's genome—in particular their alleles for ion channels of the invention—to determine whether the individual possesses a genetic characteristic found in other individuals that are considered to be afflicted with, or at risk for, developing a mental disorder or disease of the brain that is suspected of having a hereditary component. For example, in one embodiment, the invention provides a method for determining a potential for developing a disorder affecting the brain in a human subject comprising the steps of analyzing the coding sequence of one or more ion channel genes from the human subject; and determining development potential for the disorder in said human subject from the analyzing step.

More particularly, the invention provides a method of screening a human subject to diagnose a disorder affecting the brain or genetic predisposition therefor, comprising the steps of: (a) assaying nucleic acid of a human subject to determine a presence or an absence of a mutation altering the amino acid sequence, expression, or biological activity of at least one ion channel that may be expressed in the brain, wherein the ion channel comprises an amino acid sequence selected from the group consisting of SEQ ID NO:52 to SEQ ID NO:102, and SEQ ID NOS: 105, 106, 109, and 110, or an allelic variant thereof, and wherein the nucleic acid corresponds to the gene encoding the ion channel; and (b) diagnosing the disorder or predisposition from the presence or absence of said mutation, wherein the presence of a mutation altering the amino acid sequence, expression, or biological activity of allele in the nucleic acid correlates with an increased risk of developing the disorder.

By “human subject” is meant any human being, human embryo, or human fetus. It will be apparent that methods of the present invention will be of particular interest to individuals that have themselves been diagnosed with a disorder affecting the brain or have relatives that have been diagnosed with a disorder affecting the brain.

By “screening for an increased risk” is meant determination of whether a genetic variation exists in the human subject that correlates with a greater likelihood of developing a disorder affecting the brain than exists for the human population as a whole, or for a relevant racial or ethnic human sub-population to which the individual belongs. Both positive and negative determinations (i.e., determinations that a genetic predisposition marker is present or is absent) are intended to fall within the scope of screening methods of the invention. In preferred embodiments, the presence of a mutation altering the sequence or expression of at least one ion-x ion channel allele in the nucleic acid is correlated with an increased risk of developing the disorder, whereas the absence of such a mutation is reported as a negative determination.

The “assaying” step of the invention may involve any techniques available for analyzing nucleic acid to determine its characteristics, including but not limited to well-known techniques such as single-strand conformation polymorphism analysis (SSCP) [Orita et al., Proc Natl. Acad. Sci. USA, 86: 2766-2770 (1989)]; heteroduplex analysis [White et al., Genomics, 12: 301-306 (1992)]; denaturing gradient gel electrophoresis analysis [Fischer et al., Proc. Natl. Acad. Sci. USA, 80: 1579-1583 (1983); and Riesner et al., Electrophoresis, 10: 377-389 (1989)]; DNA sequencing; RNase cleavage [Myers et al., Science, 230: 1242-1246 (1985)]; chemical cleavage of mismatch techniques [Rowley et al., Genomics, 30: 574-582 (1995); and Roberts et al., Nucl. Acids Res., 25: 3377-3378 (1997)]; restriction fragment length polymorphism analysis; single nucleotide primer extension analysis [Shumaker et al., Hum. Mutat., 7: 346-354 (1996); and Pastinen et al., Genome Res., 7: 606-614 (1997)]; 5′ nuclease assays [Pease et al., Proc. Natl. Acad. Sci. USA, 91:5022-5026 (1994)]; DNA Microchip analysis [Ramsay, G., Nature Biotechnology, 16: 40-48 (1999); and Chee et al., U.S. Pat. No. 5,837,832]; and ligase chain reaction [Whiteley et al., U.S. Pat. No. 5,521,065]. [See generally, Schafer and Hawkins, Nature Biotechnology, 16: 33-39 (1998).] All of the foregoing documents are hereby incorporated by reference in their entirety.

Thus, in one preferred embodiment involving screening ion-x sequences, for example, the assaying step comprises at least one procedure selected from the group consisting of: (a) determining a nucleotide sequence of at least one codon of at least one ion-x allele of the human subject; (b) performing a hybridization assay to determine whether nucleic acid from the human subject has a nucleotide sequence identical to or different from one or more reference sequences; (c) performing a polynucleotide migration assay to determine whether nucleic acid from the human subject has a nucleotide sequence identical to or different from one or more reference sequences; and (d) performing a restriction endonuclease digestion to determine whether nucleic acid from the human subject has a nucleotide sequence identical to or different from one or more reference sequences.

In a highly preferred embodiment, the assaying involves sequencing of nucleic acid to determine nucleotide sequence thereof, using any available sequencing technique. [See, e.g., Sanger et al., Proc. Natl. Acad. Sci. (USA), 74: 5463-5467 (1977) (dideoxy chain termination method); Mirzabekov, TIBTECH, 12: 27-32 (1994) (sequencing by hybridization); Drmanac et al., Nature Biotechnology, 16: 54-58 (1998); U.S. Pat. No. 5,202,231; and Science, 260: 1649-1652 (1993) (sequencing by hybridization); Kieleczawa et al., Science, 258: 1787-1791 (1992) (sequencing by primer walking); (Douglas et al., Biotechniques, 14: 824-828 (1993) (Direct sequencing of PCR products); and Akane et al., Biotechniques 16: 238-241 (1994); Maxam and Gilbert, Meth. Enzymol., 65: 499-560 (1977) (chemical termination sequencing), all incorporated herein by reference.] The analysis may entail sequencing of the entire ion-x gene genomic DNA sequence, or portions thereof; or sequencing of the entire receptor coding sequence or portions thereof. In some circumstances, the analysis may involve a determination of whether an individual possesses a particular allelic variant, in which case sequencing of only a small portion of nucleic acid—enough to determine the sequence of a particular codon characterizing the allelic variant—is sufficient. This approach is appropriate, for example, when assaying to determine whether one family member inherited the same allelic variant that has been previously characterized for another family member, or, more generally, whether a person's genome contains an allelic variant that has been previously characterized and correlated with a mental disorder having a heritable component.

In another highly preferred embodiment, the assaying step comprises performing a hybridization assay to determine whether nucleic acid from the human subject has a nucleotide sequence identical to or different from one or more reference sequences. In a preferred embodiment, the hybridization involves a determination of whether nucleic acid derived from the human subject will hybridize with one or more oligonucleotides, wherein the oligonucleotides have nucleotide sequences that correspond identically to a portion of the ion-x gene sequence taught herein, or that correspond identically except for one mismatch. The hybridization conditions are selected to differentiate between perfect sequence complementarity and imperfect matches differing by one or more bases. Such hybridization experiments thereby can provide single nucleotide polymorphism sequence information about the nucleic acid from the human subject, by virtue of knowing the sequences of the oligonucleotides used in the experiments.

Several of the techniques outlined above involve an analysis wherein one performs a polynucleotide migration assay, e.g., on a polyacrylamide electrophoresis gel (or in a capillary electrophoresis system), under denaturing or non-denaturing conditions. Nucleic acid derived from the human subject is subjected to gel electrophoresis, usually adjacent to (or co-loaded with) one or more reference nucleic acids, such as reference ion channel-encoding sequences having a coding sequence identical to all or a portion of a sequence selected from the group consisting of SEQ ID NO:1 to SEQ ID NO:51 and SEQ ID NOS:103, 104, 107, and 108, (or identical except for one known polymorphism). The nucleic acid from the human subject and the reference sequence(s) are subjected to similar chemical or enzymatic treatments and then electrophoresed under conditions whereby the polynucleotides will show a differential migration pattern, unless they contain identical sequences. [See generally Ausubel et al. (eds.), Current Protocols in Molecular Biology, New York: John Wiley & Sons, Inc. (1987-1999); and Sambrook et al., (eds.), Molecular Cloning, A Laboratory Manual, Cold Spring Harbor, N.Y.: Cold Spring Harbor Laboratory Press (1989), both incorporated herein by reference in their entirety.]

In the context of assaying, the term “nucleic acid of a human subject” is intended to include nucleic acid obtained directly from the human subject (e.g., DNA or RNA obtained from a biological sample such as a blood, tissue, or other cell or fluid sample); and also nucleic acid derived from nucleic acid obtained directly from the human subject. By way of non-limiting examples, well known procedures exist for creating cDNA that is complementary to RNA derived from a biological sample from a human subject, and for amplifying DNA or RNA derived from a biological sample obtained from a human subject. Any such derived polynucleotide which retains relevant nucleotide sequence information of the human subject's own DNA/RNA is intended to fall within the definition of “nucleic acid of a human subject” for the purposes of the present invention.

In the context of assaying, the term “mutation” includes addition, deletion, and/or substitution of one or more nucleotides in the ion-x gene sequence (e.g., as compared to the ion channel-encoding sequences set forth of SEQ ID NO:1 to SEQ ID NO:51 and SEQ ID NOS:103, 104, 107, and 108) and other polymorphisms that occur in introns (where introns exist) and that are identifiable via sequencing, restriction fragment length polymorphism, or other techniques. The various activity examples provided herein permit determination of whether a mutation modulates activity of the relevant receptor in the presence or absence of various test substances.

In a related embodiment, the invention provides methods of screening a person's genotype with respect to ion channels of the invention, and correlating such genotypes with diagnoses for disease or with predisposition for disease (for genetic counseling). For example, the invention provides a method of screening for an ion-x mental disorder genotype in a human patient, comprising the steps of: (a) providing a biological sample comprising nucleic acid from the patient, the nucleic acid including sequences corresponding to said patient's ion-x alleles; (b) analyzing the nucleic acid for the presence of a mutation or mutations; (c) determining an ion-x genotype from the analyzing step; and (d) correlating the presence of a mutation in an ion-x allele with a mental disorder genotype. In a preferred embodiment, the biological sample is a cell sample containing human cells that contain genomic DNA of the human subject. The analyzing can be performed analogously to the assaying described in preceding paragraphs. For example, the analyzing comprises sequencing a portion of the nucleic acid (e.g., DNA or RNA), the portion comprising at least one codon of the ion-x alleles.

Although more time consuming and expensive than methods involving nucleic acid analysis, the invention also may be practiced by assaying protein of a human subject to determine the presence or absence of an amino acid sequence variation in ion channel protein from the human subject. Such protein analyses may be performed, e.g., by fragmenting ion channel protein via chemical or enzymatic methods and sequencing the resultant peptides; or by Western analyses using an antibody having specificity for a particular allelic variant of the ion channel.

The invention also provides materials that are useful for performing methods of the invention. For example, the present invention provides oligonucleotides useful as probes in the many analyzing techniques described above. In general, such oligonucleotide probes comprise 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides that have a sequence that is identical, or exactly complementary, to a portion of a human ion channel gene sequence taught herein (or allelic variant thereof), or that is identical or exactly complementary except for one nucleotide substitution. In a preferred embodiment, the oligonucleotides have a sequence that corresponds in the foregoing manner to a human ion channel coding sequence taught herein, and in particular, the coding sequences set forth in SEQ ID NO:1 to SEQ ID NO:51. In one variation, an oligonucleotide probe of the invention is purified and isolated. In another variation, the oligonucleotide probe is labeled, e.g., with a radioisotope, chromophore, or fluorophore. In yet another variation, the probe is covalently attached to a solid support. [See generally Ausubel et al. and Sambrook et al., supra.]

In a related embodiment, the invention provides kits comprising reagents that are useful for practicing methods of the invention. For example, the invention provides a kit for screening a human subject to diagnose a mental disorder or a genetic predisposition therefor, comprising, in association: (a) an oligonucleotide useful as a probe for identifying polymorphisms in a human ion-x ion channel gene, the oligonucleotide comprising 6-50 nucleotides that have a sequence that is identical or exactly complementary to a portion of a human ion-x gene sequence or ion-x coding sequence, except for one sequence difference selected from the group consisting of a nucleotide addition, a nucleotide deletion, or nucleotide substitution; and (b) a media packaged with the oligonucleotide containing information identifying polymorphisms identifiable with the probe that correlate with a mental disorder or a genetic predisposition therefor. Exemplary information-containing media include printed paper package inserts or packaging labels; and magnetic and optical storage media that are readable by computers or machines used by practitioners who perform genetic screening and counseling services. The practitioner uses the information provided in the media to correlate the results of the analysis with the oligonucleotide with a diagnosis. In a preferred variation, the oligonucleotide is labeled.

In still another embodiment, the invention provides methods of identifying those allelic variants of ion channels of the invention that correlate with mental disorders. It is well known that ion channels, including ion-x, are expressed in many different tissues, including the brain. Accordingly, the ion-x of the present invention may be useful, inter alia, for treating and/or diagnosing mental disorders. For example, the invention provides a method of identifying an ion channel allelic variant that correlates with a mental disorder, comprising steps of: (a) providing a biological sample comprising nucleic acid from a human patient diagnosed with a mental disorder, or from the patient's genetic progenitors or progeny; (b) analyzing the nucleic acid for the presence of a mutation or mutations in at least ion channel that is expressed in the brain, wherein the ion channel comprises an amino acid sequence selected from the group consisting of SEQ ID NO:1 to SEQ ID NO:51, and SEQ ID NOS:103, 104, 107, and 108, or an allelic variant thereof, and wherein the nucleic acid includes sequence corresponding to the gene or genes encoding the ion channel; (c) determining a genotype for the patient for the ion channel from said analyzing step; and (d) identifying an allelic variant that correlates with the mental disorder from the determining step. To expedite this process, it may be desirable to perform linkage studies in the patients (and possibly their families) to correlate chromosomal markers with disease states. The chromosomal localization data provided herein facilitates identifying an involved ion channel with a chromosomal marker.

The foregoing method can be performed to correlate ion channels of the invention to a number of disorders having hereditary components that are causative or that predispose persons to the disorder. For example, in one preferred variation, the ion channel comprises ion-75 having an amino acid sequence set forth in SEQ ID NO:82 or an allelic variant thereof.

Also contemplated as part of the invention are polynucleotides that comprise the allelic variant sequences identified by such methods, and polypeptides encoded by the allelic variant sequences, and oligonucleotide and oligopeptide fragments thereof that embody the mutations that have been identified. Such materials are useful in in vitro cell-free and cell-based assays for identifying lead compounds and therapeutics for treatment of the disorders. For example, the variants are used in activity assays, binding assays, and assays to screen for activity modulators described herein. In one preferred embodiment, the invention provides a purified and isolated polynucleotide comprising a nucleotide sequence encoding an ion channel allelic variant identified according to the methods described above; and an oligonucleotide that comprises the sequences that differentiate the ion-x allelic variant from the sequences set forth in SEQ ID NO:1 to SEQ ID NO:51 and SEQ ID NOS:103, 104, 107, and 108. The invention also provides a vector comprising the polynucleotide (preferably an expression vector); and a host cell transformed or transfected with the polynucleotide or vector. The invention also provides an isolated cell line that is expressing the allelic variant ion channel polypeptide; purified cell membranes from such cells; purified polypeptide; and synthetic peptides that embody the allelic variation amino acid sequence. In one particular embodiment, the invention provides a purified polynucleotide comprising a nucleotide sequence encoding a ion-31 protein of a human that is affected with a mental disorder; wherein said polynucleotide hybridizes to the complement of SEQ ID NO:1 under the following hybridization conditions: (a) hybridization for 16 hours at 42° C. in a hybridization solution comprising 50% formamide, 1% SDS, 1 M NaCl, 10% dextran sulfate and (b) washing 2 times for 30 minutes at 60° C. in a wash solution comprising 0.1×SSC and 1% SDS; and wherein the polynucleotide encodes a ion-31 amino acid sequence that differs from SEQ ID NO:52 by at least one residue.

An exemplary assay for using the allelic variants is a method for identifying a modulator of ion-x biological activity, comprising the steps of: (a) contacting a cell expressing the allelic variant in the presence and in the absence of a putative modulator compound; (b) measuring ion-x biological activity in the cell; and (c) identifying a putative modulator compound in view of decreased or increased ion-x biological activity in the presence versus absence of the putative modulator.

Additional features of the invention will be apparent from the following Examples. Examples 1, 2, 12 and portions of Example 3 are actual, while the remaining Examples are prophetic. Additional features and variations of the invention will be apparent to those skilled in the art from the entirety of this application, including the detailed description, and all such features are intended as aspects of the invention. Likewise, features of the invention described herein can be recombined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

Table 5 contains the sequences of the polynucleotides and polypeptides of the invention, in addition to exemplary primers useful for cloning said sequences. “X” indicates an unknown amino acid or a gap (absence of amino acid(s)).

TABLE 5

The following DNA sequence Ion31 was identified in H. sapiens:

<SEQ ID NO. 1>

CCCTCCTCCCTGGCCCCGGGTGCCCTTTCTCCTCCTGAAGTGGGAGGAGCCATACTGATGAGGGGGCT
GCCACTGGCAGGGGAGCAAGTCATTCATCATGAGCAGGAAGACGTTGTAGCCCAGCAGAAGTGTTATC
TTGAATGGGGCACGATTCTCGCTCTCTGCTGGCAGGTAGAAGCTGAGGGCATCAATGGCAACCAGAAA
GCTACTGGGCACCAGCAGGTTTATGATGTAGAGCCTTGGCCTGCGCCTGATGGCCACCTGGACAGAGA
CCGGAGAAGATGGCAAATAAATAGATGTCAGAGGGCTCAATTTGTATATCTGACCCCTAATCTTTGCC
AATGTGCTGTGAGGCTCCTGGGGACGATCTTTTTAAGTAACACTTTTGCATATAATTGTGCTCGCCTA
CATAGGGGCCTCTGATTTGTTGTCTAATTTTTATTCATTTTTAACCTACTAGGAACACAATGACTGTA
GAATTTTAGGTGCAAGTGGGCCCTTTAAGTCATTCTGAGCAGTAGGGGTGAGCTGATCCATTCTGAGC
AGCAGGCCTTATTACAGTCCAGCCATTCCT

The following amino acid sequence <SEQ ID NOS. 52> is a predicted
amino acid sequence derived from the DNA sequence of SEQ ID NO. 1:

<SEQ ID NO: 52>

VAIRRRPSLYIINLLVPSSFLVAIDALSFYLPAESENRAPFKITLLLGYNVFLLMMNDLL

The following DNA sequence Ion32 was identified in H. sapiens:

<SEQ ID NO. 2>

AGAAGAAAACAGTGACTGGTCCCAAGTAAGTCTGAAACCCAACAGGAGGAAACAACATGAAATGTTAC
GGCTTGAGAATAATCATTGGCTCAATGTTCTGCCCTCCAGGCCCACTAAGGTGATAGTGCCACCTTCA
GGACACAATGTGGTAGCAGCCCTGCCCTGTGGCTTTGGGTGGCCTTGCCCCTGAGGCAGCCATGTGCT
TGTGCCTTGCTCCTGCCTCCACAGTAGCTCTGTGCCTGGGTCATCCATCTGAGGCTCTCCTGGAAACC
ACCACTGGTGACTCTACTGGTCTGGACTCATGGGCCTAGTGCGGGCCCTCTGTAGTGGCCCTTCCCCA
ACAGTGATTCTCCGTCTCAGCCCCATGGCTCTCTTGGGGCATCCTTTTAATTCTGGGGGAAGGCAGCC
ATGCCCCCACATCTTTCTACTCAAGGGCCCGTGATGGGATGGGTAGCTGTGATGA

The following amino acid sequence <SEQ ID NO. 53> is a predicted
amino acid sequence derived from the DNA sequence of SEQ ID NO. 2:

<SEQ ID NO: 53>

SAPWLSWGILLILGEGSHAPTSFYSR

The following DNA sequence Ion33 was identified in H. sapiens:

<SEQ ID NO. 3>

TATACTATACATAAAACAATTAGAGAACAACTAAGTGCTAAATTAAGTTTTCTGGCAATGGTTTCTGA
TTATATATTTGTTTGATTTTTAAGGTATACATGCATGTAGTTTCAGAGTTAGAAGGCAAAGTAGTTCT
ATAAACCTTGTAACAAAAATAGCAATTCTTGAGGCCTGCATCATCTTAGGTTCAGCTTTTCAGAGGCA
ACACTTTAAATTGTTTCAGCTGGTAATGTTCTAGGACTGTACCTCCATATCTCTATAACACAGATGTA
TGGTTTTTTTTTATTAGGCATTATCCATGGACTTTTCATTATGAAAGATGAAGATTTCTGCCCTAACC
CCACACCCCTACTCCCCACCACACACAATTGTCTTCT

The following amino acid sequence <SEQ ID NO. 54> is a predicted
amino acid sequence derived from the DNA sequence of SEQ ID NO. 3:

<SEQ ID NO: 54>

RTVPPYLYNTDVWFFFIRHYPW

The following DNA sequence Ion34 was identified in H. sapiens:

<SEQ ID NO. 4>

CCGGCCTTCCCGTGCCCTCACAGTCCTCCTCCTCAGCTGTTTCAGCTAAAGTCCCAGGATTAATGCTT
ATTGGCTGGCTTGGGCCTGAACTGAACTCCCTGAACTGAGGCTAGCAGGATGAAATGCTCTAATCAGC
CAGATGTGAGTCATTCACCCCCTCCTGGAGCCTGGGGCTGAGGACCTGTGGGTGTCAACCCTGCCAAG
TGACCTGGACAGAACACAGAGGAGCAGAAACTCCCCAGAGGGAAACTGAGGAGGTGGGGGTGGAGGGA
CACAGAGCCAGCAGGGCCACCGGAAGGAGGCCCTTGCATTTCTGCACATCCACCCAGCCAGGAGGAGA
CAGCTAGGCCCAGGGGTTGCGGCAGTGCGTGCAAGGCGTTTTCTTGCAGGAGAGGCTGGTCTTTACAG
GGGACAGGAAATGTGGGTGAACTCAGCCGTTTTCTTTGCGGGGGCAGAATGTACAGGCTGATACAGTG
ACCGCAGAAGCTGCTGGGTCCCCCTGTGGGGTTTTCTGCCAGTGAAGACCCCCGTCTGCCCCCCTGCA
CAGCTCCTTGGGCTTCCAAATATTTTGTCTGTGCTGACAGCTTT

The following amino acid sequence <SEQ ID NO. 55> is a predicted
amino acid sequence derived from the DNA sequence of SEQ ID NO. 4:

<SEQ ID NO: 55>

GGRRGSSLPQNPTGGPSSFCGNCISLYILPPQR

The following DNA sequence Ion35 was identified in H. sapiens:

<SEQ ID NO. 5>

CCTTTCATCAATTCTTGAAAATTCTTAGGCTTTATGTTTCAAATATTGCCTCTTCTCTTTTTCTTTTA
CTTTTGGGAAACTCCCATTATGTATATGATGGACTTTCTTATTCTGTCTTTCCTATCTTTTTCCATAT
TTTCCACTTTTTATATTGGTCTCCTTTTTCTTAGAATTTCTCAAATCTCT

The following amino acid sequence <SEQ ID NO. 56> is a predicted
amino acid sequence derived from the DNA sequence of SEQ ID NO. 5:

<SEQ ID NO: 56>

LLLLGNSHYVYDGLSYSVFPIFFHIFHFLYWSPFS

The following DNA sequence Ion36 was identified in H. sapiens:

<SEQ ID NO. 6>

TCTGTGTGTTTACCCAGGGGACTGCCGCATGGCCCATGCCGAGCAGAAACTGATGGACGACCTTCTGA
ACAAAACCTGTTACAACAACCTGGATCCGCCCAGCCACCAGCTCCTCACAGCTCATCTCCATCCAGAC
GGCGCTCTCCCTGGCCCAGTGCATCAGCGTGGTAGGTGCAGAGGGTACCTGTGGCTCAGGCTCAGGTG
AAGAGGCAGCTCATGCCCAAGCCCTAAGCAGTCAATGTCCAGAGGAATGAAATGACTAGAGTTGA

The following amino acid sequence <SEQ ID NO. 57> is a predicted
amino acid sequence derived from the DNA sequence of SEQ ID NO. 6:

<SEQ ID NO: 57>

GDCRMAEAEQKLMDDLLNKTCYNNLDPPSHQLLTAHL

The following DNA sequence Ion37 was identified in H. sapiens:

<SEQ ID NO. 7>

CCCTTGTGATTCAGACATCTGCCCTGGGACCCACAGTAGGTTCCGACGGAGACGTTCAGCCTGGGCTG
GCCTGGGGATAGCCTAAAGTGGGGGTGGCATGGGAGGGGCTGAGTGCTTGGCAGCTTAGAAGGGTCCT
GGGGAAAAGCTTCCAGGGCAGCGTGGCAACCAGGTTATGTGGTAGGGAGAGGGGATCACTACACCCCC
ACAGCTAAGGGCAAGTCTAGAGAGGGGTAAGAGAGAGGAGGGGCCCAGATAGGCAGTACTTGTTATAG
AGTACGATGTCTGGCCGCCACACAAGACTGCTGGGGATGCGGATGGCATCCAGGCCACCATAGGCATT
GGGGTCCCATCCTAGGTAGGCATCTGTCCACTCCTGCCGTATCCACAGATACAGGGTCAGCACCTGGT
TCCGTTCATCCTAGTGGGGGCAGGGAATGGCAGAGATGTGGACATGTATATGCATATCCTGCCCTGTC
TGTGCACACTCCCCTGCAGGGCTCTGGTCAGCACCCACAAACCTGACTTCTCCATACCGTCCAGTTCC
CACCCAGACCTGACCTTGCCATGTGACCTTAGTGGGCTCTTCTCTTTTCTGCCCGTTTCCTCAGCAGG
AATATGGGGTGAGAATCCCTGCTTA

The following amino acid sequence <SEQ ID NO. 58> is a predicted
amino acid sequence derived from the DNA sequence of SEQ ID NO. 7:

<SEQ ID NO:58 >

DERNQVLTLYLWIRQEWTDAYLRWDPNAYGGLDAIRIPSSLVWRPDIVLYNK

The following DNA sequence Ion38 was identified in H. sapiens:

<SEQ ID NO. 8>

CCTCTTAGTTTGTAATCAGCCACCCTATTTTTTTTTTTTTCCAAAAGCAAATTGTTCTTTGCAAGAAC
AATTCTATTGACTTCAAATTACTCTTGCTATAGGTCCTTTCGAAGTATCGTCATGACATGTACACACA
GACTTGAGGGAAAAAAGTGCTTTTCTGAAAAAGTAATGATTGAAATTTTTATTTTAAATGATTCCTTA
GATTGAATTCACTTTAGATTAACAGATTTTCCTGCCCAATTGATTTTCTGGCATCCATGCAGTGATCC
AGCAGAGATAAAATGGGGGTTCATTTAGTCCATGGCTCCAAGGAAAAGTGAGAGCCTGGCAAAGAGAG
CCAGCAAAGCTTCTTTCTTGCCTGCTCGGTTGGAGCAGGACAACTGGAGCCGGTCAGCTGCTGACCAG
ATGCTGCCTTCAATTAATATTCCAACCCTCAAAGACATTTATCGCTTACTCTCGAAAGCAGAGCAGCT
GAGTAATAAACGGAACCACTAAAGCTGTTTTTTTTTCAAGAGCATTTATAATGGCTAAATTGCTTGAA
ATAAATTAGCACGAAAAATAAACATAGTTTGTACAGTATCTGTAAAACAAATTTCCAATCTTGGGAAA
ATAGAGCGACAAAGTGGGAGCTTGCATT

The following amino acid sequence <SEQ ID NO. 59> is a predicted
amino acid sequence derived from the DNA sequence of SEQ ID NO. 8:

<SEQ ID NO: 59>

HFVALFSQDWKFVLQILYKLCLFFVLI

The following DNA sequence Ion39 was identified in H. sapiens:

<SEQ ID NO. 9>

CAAGTGCAGGCCAATATATTCGTGTGGTCCTAACTCCAAGTGGTTTTAGGCCATATTTAATATCTGTC
TGCCAAAAGGCTATCAACGGGTACTTTTCTGGTGACACCTTGTTAAATCAAGAATGGGGGGATAGGCT
GTGGTTATTAGGGTCACAAATGGGAGTGGGAGGATCAAGGTTTAAAGAAGAATGGAAAGGGTGGGAGA
GGCCGACAGGACAAGCTCACCGTCACTCACCTCTGTCCCTCACTGCCCTGATGCAGGTATGGGACAAT
CCTTTCATTAATTGGAACCCAAAAGAGTGTGTTGGCATCAATAAACTCACAGTATTAGCTCAAAACCT
GTGGCTCCCAGACATCTTCATCGTGGAATCGTGCGTATGCAGGCTGGGGAAGCCAGCGTGAAACCTCA
TCTGCCGAGAACACCCTAGGGTCAGCACAGGCCATGGGGCCACCGAAAGATTCAGACAGGCACACAGT
CTCAACGAACTGACTTCCACACATCACTACGAGTAGAAGAGGCGAGAGAGTGACATTAAAGAAAGAGC
CCAGGGCCAGGCGCGGTGGCTCACGCCTG

The following amino acid sequence <SEQ ID NO. 60> is a predicted
amino acid sequence derived from the DNA sequence of SEQ ID NO. 9:

<SEQ ID NO: 60>

LNQVWDNPFINWNPKECVGINKLTVLAENLWLPDIFIVES

The following DNA sequence Ion40 was identified in H. sapiens:

<SEQ ID NO. 10>

CAATTATATACCTGTGTCTTTAATCCCTGGGAGCAGAATGATGAATATTTGAGCCCCAGTATATCATA
TATACATGTAATTAATTTTTAAAAGGTAGTTCAATATTCAAATTTATTGCAAAGTGGCCAAGAACAGT
GCAAGTGTTGACGACTTATAAATAGAACTACATTGACTATTTACATTAGGTTCTTGAGGATTGAAATA
ACATTCTTCTGTTTTTCCTAATAAATGACAGGCTTATATACATAGACTTGAGTTAAAAATTGACCAAT
ATTAACTGCCATGAGCCCGTGGTGAACAAATTATTGCTGTCATCTCAAACACAATAATTAATAGATTA
ATTACTAGGATTTACCAAAATGGCTTTTTGAAGATCTATTTTTAATGTTCTTTTCTGTTAAAAGCAGC
TTACACAAGTTTCCTAAATCTATACTGCCACTAATGATAGTACCACAGCATCTTAGTATAAAATTTCT
GGAGTTTGAATGTTTGCCCCCTCCAAAATTCACGTTGAAATTTAATTGTCATTGTAAAAGTATTAAGA
TATGAGACCTTTAAGAGGTGATTAGGCCACCCAGTATTATGGGTGGAATTAATGCCATTATGAAAGAA
TGAATTTGGTTCCCTTTTTCCTCTGTGTCCTTTGGCCATGTAATGAGACAACAAGAAAGCCCTTGTCA
GATGTCACCATTCTTTATATTGGACTTTCCAGCCTT

The following amino acid sequence <SEQ ID NO. 61> is a predicted
amino acid sequence derived from the DNA sequence of SEQ ID NO. 10:

<SEQ ID NO: 61>

REPNSFFHNGINSTHNTGWPNHLLKVSYLNTFTMTIK

The following DNA sequence Ion41 was identified in H. sapiens:

<SEQ ID NO. 11>

CTTCTTATTCTTGGACTTTATAAATATTTGAACCATCACATGTATAAGTTCAGGCCATATGTAAATAA
GACATTGTACATACTTGATTGGTTTATTATTGCCTATTGCTTTCTCCCTATCAATTCCCCCAAAATCA
GTGTTATGCAGATTTACTGTATTAAACTACAATTCCATTCCTTCATCCTTTATAGCCATATAAATTAT
ATTTCTGAGAGTAGCTAATATATGCTGTGATTCCTTAAAGTCAATATACCACAGTCTGATCCAATCTA
GGCAGAAAGATATAGTGGGTCAAATTTGGAATTTAAAACATAGGGCTTCTTCAGGTTTATTTAAGCTT
GCTAAAAAATCAAAGCCTACCAAGCTAGTTAGTCTTTCTGTGTCACACTTGCTACCAATGGAAGTTCT
CCCTTTTCAGAAGTAATAGAGGTCCACACAGTTGTCTGGAAGAAAATTGATCTTGCAAGTACATCATG
TCTATTCAACACCAAATTTACTAGGTTCAACATCGAGCATTCAATCAGAGTGTGTGTCTATAAGAACC
AAGCTCACGTTCATGTGATTATTCTGGTTGOGCCAATGAGTTGCTTGGGGCTCTGTAGGAAAGATTTA
CAGCAAAGTAGTAAGGCT

The following amino acid sequence <SEQ ID NO. 62> is a predicted
amino acid sequence derived from the DNA sequence of SEQ ID NO. 11:

<SEQ ID NO: 62>

TLIECSMLNLVNLVLNRHDVLARSIFFQTTVWTSITSEKGELPLVASVTQKD

The following DNA sequence Ion56 was identified in H. sapiens:

<SEQ ID NO. 12>

ATCAGCTGAAGGATCAAAGTCACAATTACTAGCTGTGAGTGTGCCAAGCTAACCATTTAGCACCCATG
CCACAAGCATGCTCTGTGCTACTCAGCATCATGTACACATTCTCAGAAGTGACACAAGTTGACATCAG
AAGTGTTTTGTATTTCAGATTTAGGGATTTTTCATTATAGTTATCAGTTGAGCATCTCAAATCCTGAA
AATCCAAAACACTCCAATGAGCATTTCCTTTGAGTGTCACATTGGTACTCAAAGAATTTCAGATTTTG
GAGCATTTTGGATTTTCAGGTTTGGCATGTACATTAGTCCACGTTCACACTGTTACAAAGACATACCC
AAGACTGGGTAATTTATAAAGAAAAGAGGTTTAAATGACTCACAGTTCCACATGGCTGGTGAGGCCAC
AGGAAACTTACAATCATGGCGAAAAGCACCTCTTCACAGGGCAGCAGGAGACAGAAGGGTGAGGAGCA
AAGGGGGAGGAGCCCCTTATAAAACCATCAGATCTCCTGAGAACTCCCTCGTTATCACAAGAACAGCA
TGGGGGAAATCACCCCCATGATCCCATCTCCTAGGATTCTACTGGATCCAGCACTGTCCAATAGATTT
TTTTTTT

The following amino acid sequence <SEQ ID NO. 63> is a predicted
amino acid sequence derived from the DNA sequence of SEQ ID NO. 12:

<SEQ ID NO: 63>

CISDLGIFHYSYQLSISNPENPKHSNEHFLVSHWYSKNPRFW

The following DNA sequence Ion57 was identified in H. sapiens:

<SEQ ID NO. 13>

GACCATTTAGGTGGCTATGGTCATAATCATGAAAGCTTGGACACAGTGGTGGTGGTGCAGGTGATGAG
GTTTGGAGCAAAGGATGACGTGATCTGACTGAGGCTTAATAGGATCATTCTGGTTTCTGGGGATGAGA
AAGTAAAATTTGTAGATATTTTGAAGCATTTTCTGTTGGCCTGAATCGCAGGAGTATGTGTGGAAAAG
GAAGAAGGAATCCATAGACTTGCTATTTGAGTTTAGAAAACGTTTTGGCCTCATCAAGGTATACTCGG
TCACTGGGCGTGTGAAAAAAGATGGCCGAGGGAGAATTCCTAGAAGGGGAAAATAGGGAGGGAGGACA
TGGGAGGATAACAGACTCCTAAATACATGTGGTTGAGTTCATTGGTTGTGCATATGGAAAATACCCCT
ACCTCAAACCATCACACAAATGATGAATTTAAGATATCAG

The following amino acid sequence <SEQ ID NO. 64> is a predicted
amino acid sequence derived from the DNA sequence of SEQ ID NO. 13:

<SEQ ID NO: 64>

SSHVLPPYFPLLGILPRPSFFTRPVTEYTLMRPKPFLNSNSKSMDSFFLPHTYSCHS

The following DNA sequence Ion58 was identified in H. sapiens:

<SEQ ID NO. 14>

ACTCCTGGAATCCTAGCCCGGACCCTGAGCCATTCAACTCAAGCAGCCCCTGAGACTAACATAGGGAG
CTGCCTGGAGACTTCCCACAGTATTCATTCTGAGAGGAACCTCACACAGGGTCCTAGACAGCTCCTAA
ATCCTAAGCAGCTACAGGAAGGCACCATTTTGAGAACACAGCCCCTATCATACTGTATTCTGCTGGAG
GGCCCAATAGCCCCTGTATCTTCACATCCCTGGAGCCCCATTGACATTCTCCACCTTTATTCACCACC
GCAGCTGGCTCTGCTGCCAAGGCCAAAATGCAAGCCATTGTCAGTAACCCAGCTGCCTCCAGTAGCAG
GGCCACTGTGCATTTAAAGGCATCCCAAAAAAAGGCTATCTCACTTATAGCAGCCACCTGAGGCCAAA
ATGTGTGCTCCCCAGCCACCTTACTGTTGCCACTGAAAGCAACCCTGCCCTCCCTAGCAGCAGGGTCC
TGGCACAGCTGCTGCTGCTCCCACCCAGGCATTCTGCCAATGGCCTGGGATCACTACATTCCGGGCTA
CCA

The following amino acid sequence <SEQ ID NO. 65> is a predicted
amino acid sequence derived from the DNA sequence of SEQ ID NO. 14:

<SEQ ID NO: 65>

PETNIGSCLETSHSIHSERKLTQGPRQLLNPKQLQEGTILRTQPLSYCILLEGPIAPVSSHPWSPIDI
LHLYSPPQLALLPRPKCKPLSVTQLPPVA

The following DNA sequence Ion59 was identified in H. sapiens:

<SEQ ID NO. 15>

CTGGGCAAGCTTTAAAGTTTGGGATTTTCCACTGTCCTTTCCGGTGCGAGCATTTATTGAATTTTGCA
GTAGTCTCCATAATTTACTGAGGAGCTACAGGAGGAGAAACAGAAAACAGTTAGGATATGCCATGCTT
TCCAAGAGGAACTGGCAAGTGCAGTGAGGATGCATTTAAACAAACCAGTGTGAGGATAGATCTCTCTA
CGTTATGCAGATCCACTTTCCATTTCTAAGCAAGTTGAACAGCAAATTTCAGTTGATGGGAACCTATA
TTTGATTATTTTAAAATAGGAAAACAGTGATTACATTTATAACAGTGTAAAATTGGTAATGTATTATT
TATAATTATTATAATCATGTGTTTCCAATCCACCAAAAGAATATGTACCAATTTGGCCAACTATCACT
AAAATACTCTTAACTCTATAGTAAATCAACAAGGTTTTATTCAAGCTAATTACAACCCCCCCCCTTTT
TTTTTTTTTAGCACTTTGCAAACTTTAGGACTGTGCTTGTGTGTGGTATACACATTGAAATAAACAGG
GTAATTTATTGTATTCTAACAATGGCTCCTTCTCTCCTCCTCCCTATGGAGGAATCCCCGGCAAGGAG
GAGTGAAAGGGTCTATGAGTGCTGCAAAGAGCCCTACCCCGATGTCACCTTTCACAGTGACCATGCGC
CGCAGGACGCTCTACTATGG

The following amino acid sequence <SEQ ID NO. 66> is a predicted
amino acid sequence derived from the DNA sequence of SEQ ID NO. 15:

<SEQ ID NO: 66>

PARRSERVYECCKEPYPDVTF

The following DNA sequence Ion60 was identified in H. sapiens:

<SEQ ID NO. 16>

CAGCGCATCGTCAGGTCCCCCCGCGCCCCCGCTGCTCACCGATGAGCGGCACGCTCTCGGCCGGTGGC
ATGCTCTCGGCCAGCAGCAACTGGGAAGACGGTGAGCGCCAGCAGCACGGTGACGCCCAGCGACACCT
TCTCGCCTGAGTCGGCAGGCAGGTGGAAGGCGAGCGGCGCAAGCAGCGAGATGAGCACGCAGGGCAGC
AGCAGGTTGCACACGTAGGGCGGCGGCGCGGCGGCGCAGCAGCAGCGTGAAGGTGACGTCGGGGTAGG
GCTCGGAGCAGCAGCCGTAGGTGAGCACGCGCCGCCGCGCCGGCATGCCCAGCACGCGCCACTCCACG
TTCTCCACGAAGTCCGCCAGGCTGGCTGCAGCGCCGCGCGGCCGCACATCCAGTTGGTGCCCGCCGTG
AGTCCAGGAGCCGAACGTCAGGCCGCAGTGCTGGGCGTCGAACGGGAAGGCTGCTACATCCACGCGGC
ACGAGCTGCGCGTGATGGCCGGCGCGTTCCCAGCGCACGGCGCCATCGTCGCGCAGGACCACGTTGGT
GCTGGCGGAACCTGGAGGCTGCG

The following amino acid sequence <SEQ ID NO. 67> is a predicted
amino acid sequence derived from the DNA sequence of SEQ ID NO. 16:

<SEQ ID NO: 67>

NAPAITRSSCRDVAAFPFDAQHCGLTFGSWTHGGHQLDVRPRGAAASLADFVENVEWRVLGMPARRRV
LTYGCCSEPYPDVTFT

The following DNA sequence Ion61 was identified in H. sapiens:

<SEQ ID NO. 17>

CCTCCCCTAGCACTTGACCTTTATTAACTCACGTAAGCATCACCACAAACCTAGGAAGTAGGTCCTCT
GGGTATCCCATTTGTACAAAAAGGGATTCGTATCTTGCCCCAGCTCATGCCCGTCGTTATTTGAGAGC
GGGACTGTCCTGGATTGTGTATGAGTGCAGCCTCCAGCAGTGACGGGAGCAATTAGAGAGCAGTAGCT
TCTGATGACCCACGTGTAGGAATGAAGGATGGGGAGAACTCGGCCCTTACCTCCTTCCTGCTTCCATC
CATGGGGCTTGGAGGGTCTGGAGAGCTTCATGGTGGGCTTATTTCCATTTGTGCAGAGGTGGCTGGGA
AGCTCAGGAACCACAGGCTTTTGTTTTGAGTCAATTGGCTTTCTCTCTCTCTTGCAGGGAAGTACTAC
TGGCCACTATGACCATGGTCACATTCTCAACAGCACTCACCATCCTTATCATGAACCTGCATTACTGT
GGTCCCAGTGTCCGCCCAGTGCCAGCCTGGC

The following amino acid sequence <SEQ ID NO. 68> is a predicted
amino acid sequence derived from the DNA sequence of SEQ ID NO. 17:

<SEQ ID NO: 68>

SLSLAGKYYNATMTMVTFSTALTILIMNLHYCGPSVRPVPAW

The following DNA sequence Ion62 was identified in H. sapiens:

<SEQ ID NO. 18>

AGGGCCGGCTGGCTCTCAAGCTGTTCCGTGACCTCTTTGCCAACTACACAAGTGCCCTGAGACCTGTG
GCAGACACAGACCAGACTCTGAATGTGACCCTGGAGGTGACACTGTCCCAGATCATCGACATGGTGCG
TTGTGGTGGTGGTACAGCTGTGGAGTCTTACCTGTCACAGTGTCAAGAAATGAAGGGGTGAGAGACTG
GGATTATTCTCCATGGAATTTCTTTTCTGTAAATGTTAATATTAACAAAGGTAGCAGTTACAAACTGT
TGGGTACTGACTGTTGGGTACTGAGTATTGGGTGCCTACCTCGTGCCCAATATTTTGTTCACCTGAAC
TTACTGAATCCCTGCTAAGCAGGGGATTCTCACCCCATATTCCTGCTGAGGAAACGGGCAGAAAAGAC
AAGAGCCCACTAAGGTCACATGGCAAGGTCAGGTCTGG

The following amino acid sequence <SEQ ID NO. 69> is a predicted
amino acid sequence derived from the DNA sequence of SEQ ID NO. 18:

<SEQ ID NO: 69>

GRLALKLFRDLFANYTSALRPVADTDQTLNVTLEVTLSQIIDM

The following DNA sequence Ion63 was identified in H. sapiens:

<SEQ ID NO. 19>

CAGTGGGATTTAGAATCCCTCGGTGAAAGTCTGGACTCTTGTGGCTTATTTGGGCCCCTCTAGCATTT
CTGGAGAGGCAGGCAGACTCCAGGTCCTTGAAAAGGGGAGGGTGGAGGAGAAATTTGTCAGCCTGGCG
CCAGAAGATAGTACCAGTTCAGTCCATGGCCTTTACCTCATGTGTCCCTGCAGGCAGGCCAGGGAGGA
ACTAGAGCCACAGCTAGAGCAAGAGAAGGCAGACACCAGGAGGACACTCATAAGGACAGGGCCCCAGC
CCTGGGAGTGGAGGGTGTGAGCAGAGGCCCTGGGACTAGGGCCTGGGATGGACAACCCTCCTTACTGA
CCCTCCAGAGTGCCTGGGAGCTGAGGGCCGGCTGGCTCTCAAGCTGTTCCGTGACCTCTTTGCCAACT
ACACAAGTGCCCTGAGACCTGTGGCAGACACAGACCAGACTCTTGAATGTGACCCTGGGAGGTGACAC
TGTCCCAGATCATTCGACATGGTGCGTTGTGGTGGGTGGTACAGCTGTGGAGTCTTACCTGTCACAGT
GTCAAGAAATGAAAGGGGTGAGAGACTGGGATTATTCTCCATGG

The following amino acid sequence <SEQ ID NO. 70> is a predicted
amino acid sequence derived from the DNA sequence of SEQ ID NO. 19:

<SEQ ID NO: 70>

AEGRLALKLFRDLFANYTSALRPVADTDQTL

The following DNA sequence Ion64 was identified in H. sapiens:

<SEQ ID NO. 20>

TTAGTGACGCCCATTATCCAAATCTTCTAACTATTCAAAAAGGGAATCCTACAAAAATAAATAATGCA
GTATTGTTTTATTGAGTTATACCTATATGCCCCACATACTCCACCAAAGATTTATTATTGATCTATCC
AGTCTCACCCATTTCTCTATTTTTCTATTTGTCTAATAAAGCAGTCCTCATTTGTTCCTTTGTCTATC
TGCCATCCGTCCGTCCTTCCTTCCTTCTTTCCACAGACTTCCTTCTACATCCCTGCCTCTGTCTTCCC
CATCATCAGTACATGACATCCCTATCTACCCATTGTTTAGACATCATCCCTACACTCACTGATTCTAC
ATTTTAATTATTTCTCAAATTCATTTACCTGGTGATTTTTCTCCATAAGCACCCTAATCCTGACCTAT
GATTCATCTCTATACTGAGAGTCTCTTCATATTGTTTTCATACTATTTATTACAACAATAATTATAAT
TAGTAACTGTGTTTAATGTCTGTGTACCACTAAACTATACCACAGCTCC

The following amino acid sequence <SEQ ID NO. 71> is a predicted
amino acid sequence derived from the DNA sequence of SEQ ID NO. 20:

<SEQ ID NO: 71>

QSHPFLYFSICLIKQSSFVPLSICHPSVLPSFFFQTSFYIPAS

The following DNA sequence Ion65 was identified in H. sapiens:

<SEQ ID NO. 21>

TTTACAATAAGCAAAGGTGACAGCAACCCAAGTGTCCACTGACAGGATGAACGGGTAAACAAAACATG
GTATATACATACAATGGGAATATTATTTAGCCTTAAAAAGGAAGGAAATTCCGACACATGCTACAATA
TTATGTTAAATCAGCAAGTCACAAAAGAACAAATACTGTATGATTTCATTTATATTAAGTACTTAGGG
TAGCCAAATTCATAGACACACAAGGTAGCATGGTGGTTGCCAGGAGCTGGGGGCAGGGGGAAACGGGA
GTTATCGTTTAATAGATAGGAAGTTTCAGTTTGGGAAGATGGAAAAAGTTATGGAGATGTATGGTGGT
GACATTTGCACAACAATATAAATATACGTAATGCCACTAAGCTGTATACTTAAGGATGGTTAAAATAG
TAAGTTTAATGTTATATATATTTAACCACAGTTTTTAAAAATCCAACTAGAGTTCATTGATTCTTTAA
GTACTTCTGTACTTTCTGAAATAAAAAGATGTTCAAGCCCTTCTTATATTTTCCTTGCCCTACTCCTG
CTGCTAGCCATTTCTTCAAGAATTCTTAGTTCCTTTTAGTAGACTCATATTTAGAAACCAAGATCTGG
ACACTAGACATGCTCATTGCT

The following amino acid sequence <SEQ ID NO. 72> is a predicted
amino acid sequence derived from the DNA sequence of SEQ ID NO. 21:

<SEQ ID NO: 72>

HYVLYCCANVTTIHLHNFFHLPKLKLPIYTITPVSPCPQLLATTMLPCVSMATLSTYKNHTVFLL

The following DNA sequence Ion66 was identified in H. sapiens:

<SEQ ID NO. 22>

CTTCTGCCTCTTTTTTACATATTTCTATTTTTAAAGTTTGTGAGTCAAAGAAGTTTTCACATATCCTA
AATGCTTATTGGAATATGTATAATTACATTTGGAATGTTGATGCATACACTTCTGTTTTTTTGTTTTT
CTTAGAGGAAAAGGTGTATTTTCCTCCATTGATTTGTGTAAATTTTTTTTCAAAAGCTTAATAAGTAT
TTTATTTTGTTTCTGTTCATTTTTATGGCATTAGGACAATTTAATAATATTCCCAGTGTAAGAAAAAC
CTCTTCTTTCAGTATAGCAAAATCCAAATAATTGAAAAGATTTTATTTGTTTTCATGTGGAGAAAGAG
GTGAGTCCTCCGATTTTATGAATCTCTTTAGTGCAGTAGGACATTAAATTTGCTCCCCTTTTCTACTT
CTTGCCATCACTAACCAATTGCCAAATGACACATCTTCCTGTTTTGTTTCCCCAGAAGCTATCTGCAT
TTTTAAGAGCATCTGTATTTGTATCTAGC

The following amino acid sequence <SEQ ID NOS. 73> is a predicted
amino acid sequence derived from the DNA sequence of SEQ ID NO. 22:

<SEQ ID NO: 73>

FSHILNAYWNMYNYIWNVDAYTSVFLFFLEEKVYFPPLICVN

The following DNA sequence Ion67 was identified in H. sapiens:

<SEQ ID NO. 23>

CCCACAAGTGTCAAAGGAAAAACGGAATAAGAATTCATTCAATAAAACAGGCCTTAAAGATGAATTTT
TTTAAAAAAAGGTAGAATAATGTTAACATGGAAAGTGAAATAGAGAGACAAAATTGAGAACTAGGCAA
AACATTACAGAGTTACCAAGTTAACCATAAAGGGAAAGGAATGTAGTAATGGCAAAGAGAAAATCCTT
GAGATAATTACTCTGAATTCAGAAAAAAAAAAAAGGAGACAAGGAATAATCACAGAGTTGATGAAAAA
GATGGAAGGCAGAGATGATACAACATAGGAATAATTGGTTTCCTTTAATTAGGGACCCATACTAATGG
AACAGAAATAAGTTTACAGAAAACTTTTCCCTAAAGGAAGGAAGAAATAAACTATATATTGAAATGAC
GTGTGGTATATAAGAAAAAAACTGATTGATAAAGAAGAATTTACATGGAAACCTCACTTCAAATAAAA
TCTGAAGACCTTCAATTGCCTCAAAGCCCAAGGTGACACATATGTCCATTGCCTCTGTGACTTCATCT
CATATTTATTCTTGGAAGAACTCACTCTTCACTGGCCATGCTTATCTTCCTTGCTGTCACTCAATATG
TCAGTGACAATAATGCCCATGGTC

The following amino acid sequence <SEQ ID NO. 74> is a predicted
amino acid sequence derived from the DNA sequence of SEQ ID NO. 23:

<SEQ ID NO: 74>

ETNYSYVVSSLPSIFFINSVIIPCLLFFFSEFRVIISRIFSLP

The following DNA sequence Ion68 was identified in H. sapiens:

<SEQ ID NO. 24>

ATAGTCAAACCAAAACTGTATATGTACTTTAACTCCCATGGTTTCCCATTCATTAAGCACAAATTGAA
TGTGGAAAGGGCCAAATATTCTATTTTTCTCCACCTACCTTCCTTTTTCAGGGTGATTTTCTTTGAGT
TTGGAGAATGGGTTCTGGAAACTGTAAAAGGCAGAAAATAATATTTATTTTACTAGTGCTGTTCTGTC
CTTCATTGGTTCCCCTAGCTAAGATTGACTGTCATTGATATTTATGAAGTTGGCATCCAAATGCTGAC
TCCATTGTGCAAAAAACAGAGAGTTTTAAGAGAACTTGTAGGATAGAAATTCACTTTAGTTTGGACTC
TCTAAATTCTCTCCTCTTAACTCTTGCCTGCAATAGTACACCACAATTTTCCCCCTTCATCAGGTGAC
CTCTTTGCATAAAATATTTAAAAGAAGGGCTTATGCTTAGCAAGAGTCCACGTGGCCTACTTTACATA
CAAAAAACTCAAAGATTCTTATTTTGTCAATTCTCTTTTCCTTCAAAAAAAAAATAATGAGAGGAAAA
GAAATCTGGCACCTCATTGGCAGAGATCACCTGC

The following amino acid sequence <SEQ ID NO. 75> is a predicted
amino acid sequence derived from the DNA sequence of SEQ ID NO. 24:

<SEQ ID NO: 75>

FFEFGEWVLETVKGRKYLFYCC

The following DNA sequence Ion69 was identified in H. sapiens:

<SEQ ID NO. 25>

ATTATGACAGTTGATCCTCATAACAACCTCTGGAGCTACATACTGGGTGCTGTTGTTATTCTCACTTT
ACAGATGAGTAAACTGAAGGTAAGAAAAGTTGAGTGCCCCGCCCAGGGTTGCAAAGCGAGGAAGTGGT
GGAGCTGGGATTGGGTGTGCCACAGTCTCTTTCTTTCGGCAGACTGAACATGCCTAGGCTCCTAATGA
TTCTGCTATCTTCCTTCCTTTCCCTGAGCCCCGGGCTGTGCAACCTGTGGCCAGCTTTCCTGACGGGG
TACATCTCAACCCTACCCCATCCCTGAAAGAAGGGGCAACACGCAACACCCATTCACTCCCTCCCAAA
TGCTGGCACTGTGCTGGGGGCTGGGCTGTGATGGTGACGGTCCCTGCCCTCGCAAAGGATACTGTGTA
TGGGCACTGCGCTGTGATGTGTTGGCTGTCATAGGCACACGCAGGAGGGAGACAGGGCTGAGGAAGTG
GAGAGAGTGAGACAGGCAAAGGGAAGCGGGAAGAGTGTTCCAGGTAGAGGGAGAGTCTGAGCAGAGGC
CCAGAGACTGAAGAGACAGGCACATCTGAGAAGCTGAAGGGAGTCCAGTGGGTACGTGCATCAGGACG
CATGCTGGGAGGTCCCTGGGGTGGGGTTATGAAAGGTGCCAAAGAGACTGAATGGCCACACAGAGCAC
ACTGAAGCCACTACAGTTGCATATTCAGAAATGCCTGAGTTCCTGGA

The following amino acid sequence <SEQ ID NO. 76> is a predicted
amino acid sequence derived from the DNA sequence of SEQ ID NO. 25:

<SEQ ID NO: 76>

EKLSAPPRVAKRGSGGAGIGCATVSFFGQTEHAAPNDSAIFLPFPEPRAVQPVASFPD

The following DNA sequence Ion70 was identified in H. sapiens:

<SEQ ID NO. 26>

CAAATACATTCAGAAAACTCTGTACTTAATTCACCTCTTTAGGACTCATAATACATATTAGCACAGTC
AAGACACTGAGAAAGTCCTGCAGTAAATAAATGTGGTTTATGTTATTTAATCCAGTGTTTTAATATTA
GGGCACTTTTGCTAAATTACTGTGTGGTAACGAATAACCTCAAAATCCCAGTGGCTTATAACCACAAA
GGTTGATTTGTTGCTCATATTTCGTGTCAGCTGTGCTTTGGCTCTGCTCCAGATGTCTTCTTCATTTC
AGATGTAGGCTAAAGGTGCAGCCTTTTTTCAGGAATATGCCATTCTTATGATAAAGGGAAAAGAGCAA
AAGCCATGCCAGACAATGTCTCCTAAAGTGTCTTGCCCAAATGTGTCATGTACCGTGTCTCTCACATT
CCATTGTCCAAAGCAAATCACATGGACAAGGCCAATGTCACTAAAATGGAAAGTCACAGAGCCTCCCA
CAGTGCAGTGCTGCCAGTCACATGGAAATGCACTGTATCTATATAATCCTCTTAGAGGAAACGAACAA
TAATGTAATAATGAAATCTGCCACAAAATACACTTATTTTTACACCAAATCTTTTTTTAATTTAATTA
CCATATGATTCAGCAATTTTACTCTTAAGTATATATTCAAAAGAACTGTAGACAAGCATTCAAATGAA
AACTTGTAATGAATGTTCATAGCAGCACTATTCATAGTAGT

The following amino acid sequence <SEQ ID NO. 77> is a predicted
amino acid sequence derived from the DNA sequence of SEQ ID NO. 26:

<SEQ ID NO: 77>

WQISLLHYCSFPLRGLYTYSAFPCDWQHCTVGGSVTFHFSDIGLVHVICFGQWNVRDT

The following DNA sequence Ion71 was identified in H. sapiens:

<SEQ ID NO. 27>

TATGGAATGAATGAATGAATGCATTGAAAGCCTACTTACCTAAAATCTCCTATATATTCAAATGATTA
ATCAAAGATCTTTCATTCAACAAAATGAACTGAGTGCATTTAGAAGGCATTGTGGGGTGAAGGAGATG
TGGCCCCTTCCTCTCTGGAGCTTAGAGTCTGTCTCCACCATTGAATCTGAAAAGCTAGCCAAATACAT
GAGTAAAAAAATTAAAATCCAAATCTTTTACCAATATAACATCGGATGACATGGCTGTAATGATCAAA
TAATTACCTGATTCTTTCCGATTCGGTTTTAAATGTTAAACATTCAGTGATGGTTAACATACTCGCTG
ATGTGAAAGGGTGGGGCCTGACTCATTACTGCGGCTAGGACAAGGGCAAATCCTGCCTCAGAACTGTC
ATTCAGAGCCTCTTGTTTGTCCTCTGTAGTCAGCTCAGTCACAGTAAGGTATGTCGTTTCTCTCAACA
TGTCATTCTTGTTTTATGTACTCAAATGCTTCCTTCTCATTGTCAACATCTGCTCTGAACTTTAAGTC
AGGCCCACTTGTTTGTAGAATAGCTCATTGACATAAAGCAAATAACACATCCCAGCCAGTCAAATCCA
AGAAACTCAGCTTTAAAAACACATTTGTATTAAAGAATTTCACTGCAAATCCATTCATTATGTTTACC
T

The following amino acid sequence <SEQ ID NO. 78> is a predicted
amino acid sequence derived from the DNA sequence of SEQ ID NO. 27:

<SEQ ID NO: 78>

WICSEILYKCVFKAEFLGFDWLGCVICFMSMSYSTNK

The following DNA sequence Ion72 was identified in H. sapiens:

<SEQ ID NO. 28>

CTCTCTTATGCTCTCCAGCAAAATAACTTCAGTGACTTTATCAGAAATGGGGTTTTAGACAGGATGTT
TCTTTGGTTAGATTTGGTATCATGTGTCTTAGGTATTTATATCTTTATCCCTTAACCATACACATACT
TTACTTGGGGTAACCTTACTAAATAAGATCTTCAATTAAGCTTAGAACTTTGTAGGATATTAGAAAGC
CAGAGTCCATATCTGTTTGTGGGGACAACTCAGACATCCCATCTTCCATTGACTATATTTTTGAGTGA
CTTTTTCGTAATTAGACTCTCTACCTTCAAATTCAGCTTCTGTGGGATCATTGATTAAA

The following amino acid sequence <SEQ ID NO. 79> is a predicted
amino acid sequence derived from the DNA sequence of SEQ ID NO. 28:

<SEQ ID NO: 79>

VLDRMFLWLDLVSCVLGIYIFIP

The following DNA sequence Ion73 was identified in H. sapiens:

<SEQ ID NO. 29>

AGCTGAGCAGAGTCTATGCAGGCCCATTGGCTGCCTAGCCAGTGGTGATCCCGCTCCCACCCTCATTT
CTTCTTTGTTAAGAAAACCATGACCTCATTAAATATTGGACACCTATAAACCTCAGGGACCTTGGTCC
AGCCTCCCCGCCACGTATTGGTGAGTCTAAGTCAACTCTGGTCATTTCATTCCTCTGGACATTGATTG
CTTGAGGCTTCGGCATGAGCTGCCTCTTCATCTGAGCCTGAGCCACAGGTGCCCTCTGCACTTACCAC
ACTGATGCACTGCGCCAGGGAGAGCTCTGTCTCGATGGAGATGAGCTGTGAGGAGCTGGTGGCTCGGC
AGATCAGGTTGTTGTAACGGGTTTTGTTCAGAAGCTCGTCCATCAGCTTCTGCTCAGCATGAGCCATG
CGGCAGTCCCCTGGGTAAACACACAGACATGCTGGGCCCTTGTGCAGCTGTCCCACACTGCAGATGAC
AGCTACAAAGCAGGAGCCAAGAGGGCCAGGGGAGCACAGGCACCCCGGGGGCCGCCTGAAGCAGTGAA
CGTGCTGGCGGACCAGGCTCTCCCTGGGGACTTCAAATGACATTCATGACAGAGCTCAGCTACTTT

The following amino acid sequence <SEQ ID NO. 80> is a predicted
amino acid sequence derived from the DNA sequence of SEQ ID NO. 29:

<SEQ ID NO: 80>

GDCRMAHAEQKLMDDLLNKTRYNNLICPATSSSQLISIETELSLAQCISVVSAE

The following DNA sequence Ion74 was identified in H. sapiens:

<SEQ ID NO. 30>

TCTGCAGGCCCATTGGCTGCCTAGCCAGTGGTGATCTCGCTCCCACCCTCATTTCTTCTTTGTTAACA
AAACCATGACCTCATTAAATACTGGACACCTATAAACCTCATGGACCCTCCTCCAGCCTCCCCACCGT
GTACCGGTGAGTCTAAGTCAACTCTAGTCATTTCATTCCTCTGGACATTGACTGCTTAGGGCTTGGGC
ATGAGCTGCCTCTTCACCTGAGCCTGAGCCACAGGTACCCTCTGCACCTACCACGCTGATGCACTGGG
CCAGGGAGAGCGCCGTCTGGATGGAGATGAGCTGTGAGGAGCTGGTGGCTGGGCGGATCAGGTTGTTG
TAACAGGTTTTGTTCAGAAGGTCGTCCATCAGTTTCTGCTCGGCATGGGCCATGCGGCAGTCCCCTGG
GTAAACACACAGACATGCTGGGCCCTTGTGCAGCTGTCTCCCACTCCAGCTGACAGCTATGAAGCAGC
AGCTGAGAGGGCCAGGGAGCACAGACACCCTGAGAGCTGGCTGAAGCAGTGAAGGTGCTGGCCGGCCT
GGCTTTCCCTGGGGACTTCAAATGACATTCACGACAGAGCTCAGCTACCTCCTCCCCATGCCATACCT
CT

The following amino acid sequence <SEQ ID NO. 81> is a predicted
amino acid sequence derived from the DNA sequence of SEQ ID NO. 30:

<SEQ ID NO: 81>

GDCRMAHAEQKLMDDLLNKTCYNNLIRPATSSSQLISIQTALSLAQCISV

The following DNA sequence Ion75 was identified in H. sapiens:

<SEQ ID NO. 31>

CTGTGAGGAGCTGGTGGCTGGGCGGATCAGGTTGTTGTAACAGGTTTTGTTCAGGAAGTCGTCCATCA
GTTTCTGCTCGGCATGGGCCATGCGGCAGTCCCCTGGGTAAACACACAGACATGCTGGGCCCTTGTGC
AGCTGTCTCCCACTGCAGCTTGACAGCTATGAAAGCAGGAGCTGAGAGGGCCAGGGAGCACA

The following amino acid sequence <SEQ ID NO. 82> is a predicted
amino acid sequence derived from the DNA sequence of SEQ ID NO. 31:

<SEQ ID NO: 82>

GDCRMAHAEQKLMDDFLNKTCYNNLIRPATSSSQ

The following DNA sequence Ion76 was identified in H. sapiens:

<SEQ ID NO. 32>

AGCTCCATCTCGATGGAGATGAGCTGTGAGGAGCTGGCGGCTGGGCGGGATCAGGTTGTGGTAACGGG
TTTTGTTCAGAAGGTCGTCCATCAGCTTCTGCTCGCCAGGGCCATGCGGCAGAACCCTGCGTAAACAC
ACAGGACCTGCTTGGTCCTTGTGCAGCTGTCCCCCACTGCAGCTGACAGCTATGAAGCAGGAGCTGAG
AGGGCCAGGGAGCACAGACACCCTGAGAGCTGGCTGAAGCAGTGAAGGGGCTGGCCGGCCTGGCTCTC
CCTGGGGACTTCAAATGACATTCATGACAGAGCTCAGCTACCTCCTCCCATGCCATACCTCTTCCTCC
TCCTCCTCCCTCAATCAATGAACAGCATCCCACGCTCTACACATCTGATACAAAACTGGCTATCTCTT
CCTGACCCCTCCCTTGGTTCATATAAGTGGCCACCAAGTCCTGTCTGTCCTCCCATCTCCACGGCTAC
AGCCATGTCCCTGCCTCCCCCGCCCTGCCCACCTTCTATTCTCTCCACCTGCACCCTGCCCCTG

The following amino acid sequence <SEQ ID NOS. 83> is a predicted
amino acid sequence derived from the DNA sequence of SEQ ID NO. 32:

<SEQ ID NO: 83>

AEQKLNDDLLNKTRYHNLIPPSRQLLTAHL

The following DNA sequence Ion77 was identified in H. sapiens:

<SEQ ID NO. 33>

AGACACCCACTTTTGTATCAGATGTGTAGAGCGTGGGATGCTGTTCATTGATCGAAGGAGGAGGAGGA
GGAAGAGGTGTGGCATGCGCGGAAGTAGCTGAGCTCTGTCATGAATGTCATTTGAAGTCCCCAGGGAG
AGCCTGGTCCGCCACCACCTTCACTGCTTCAGCCGGCCCCCGGGGTGCCTGTGCTCCCTGGCCCTCTT
GGCTCCTGCTTTGTAGCTGTCATCTGCAGTGTGGGACAGCTGCACAAGGGCCCAGCATGTCTGTGTGT
TTACCCAGGGGACTGCCGCATGGCTCATGCTGAGCAGAAGCTGATGGACCACCTTCTGAACA

The following amino acid sequence <SEQ ID NO. 84> is a predicted
amino acid sequence derived from the DNA sequence of SEQ ID NO. 33:

<SEQ ID NO: 84>

GDCRMAHAEQKLMDDLLN

The following DNA sequence Ion78 was identified in H. sapiens:

<SEQ ID NO. 34>

TCTTATTTTTCCAATGTAGTTTCTAGAACCGTTAGCACAGAAAGTTATAAACATTGTATAATTATTCA
TCTAAATGAATTGTAATAATAACTACAAAAAATTATGTCTACTGGCTGTAACATAACTTAGTAATTAT
TCTGTTTGTATGTACTTAGGTAGCTTCCAGAGGTTTATGGCTAAATGATCTCTAATAATTATTCTTAT
TTTCAAATTTAAATGTCAATTGCTCAATATATACATACAATAAAGGCTTTATAACTATGTGTATTAGT
TTGCTAGGAATGTCATAACAAAATACCATAGACTATGTGGTTTAAACAGCAGAAATGCATTTTCTCAC
AGCTTCAAAAAGGCTCTAAGTCTGGTATCAAGGTGTTAGCAAATTTGGTTTTTCCTAAGGTCTATCTT
CTTTTCTTTCAGATGGCTGCCTTCTTCCTGTGTCCTCACATGGGCTTTTCTCTGTGCATATGCATCCT
GTGTCTATGTCCAAATTTTCTTTTTAAAATAATGACCCAGTCATACTGAATGAAGGTCCACTCATATG
ATTTCATCTAAGCTTAATTACCACTTTAGAGGCCCTATTTCTAAATATGGTCATATTCTGTGGAACTG
AGAATTAGCTCTTCAACATATGAATTTTGGGGGACAAAATTCAGCATATATTTCCTGATACATAGAGC

The following amino acid sequence <SEQ ID NO. 85> is a predicted
amino acid sequence derived from the DNA sequence of SEQ ID NO. 34:

<SEQ ID NO: 85>

NLVFPKVYLLFFQMAAFFLCPHMGFSLCICILCLCPNFLFKIM

The following DNA sequence Ion79 was identified in H. sapiens:

<SEQ ID NO. 35>

TTCAATCTGCAATGTCCTTGCACTGACCAGGGCTCCATTTCCTTTATCAGAGGCTATCATGGAAATGA
TGTGGGAGTTCACCTGGCTGAGACGGAATGACTCTGTGCATGGGCTGGAAACCCTGTGGCTTGCTTAG
TACACCATACAATGGTATTTCACCTTGGACACCAGATTGCAGCAGGAGACAGGTAACTCATGTGACAA
TTTTTTTTTTTTAATTTTTACCATTGTTTTCGTAGATATTCCTAGGCCAGTTCTAAGAGTTTGTTTCT
TGGGAGATTAGTGCTGGAGGCCAGAAGTCTGAGATCAAGGTTGGTTTTTTCTGAGGCCTCTCTCCTTG
GCTTGGAAACAGCCGTTTTCTCGGTGTCTTCACATGGTCTTTTGCTCTGTACCTGTCCAAATTTCCTT
TTCTTATAAGGACATCACTTGTATAAGATAAGGGTTTTCCCCTCATTTTAACTTAATTACCTCTTTAA
AGGCCCTATCTCCAAACACAGTTACATTCCGAGGTACTGCAGGTCAGGGCTTCAGCACATGAATTTTG
GGCAAGGATGGAGAGGGTTGGAAACAATACAATTCACCCCGTAACACCAGATCTGACTCCTCTCACTA
GCCTCCT

The following amino acid sequence <SEQ ID NO. 86> is a predicted
amino acid sequence derived from the DNA sequence of SEQ ID NO. 35:

<SEQ ID NO: 86>

EFTWLRRNDSVHGLETLWLAYTIQWYFTLDTRLQQETGN

The following DNA sequence Ion80 was identified in H. sapiens:

<SEQ ID NO. 36>

ATTGCCTGCTCTGGAAGCATGCAAAGTGCACCAAATTCAGTCCAAAGGTCTGGGAGTAAATTTAGCTC
TGCCACTTACTTGCCTTGTGACCTTGGACAATGATCATCTATAAAGGAGTGATGAGAAATAGTACTAC
TTCTTTGTTATATGTTGTGTGTGTGTGTTTTGCGTGTGCGCGCATGTGTGGGTGCGCGTATTTAAAAA
GCTAAGAAATGCAAAAGGGTCAAAAGCGCTAAGCCTGGGCTCAAGAGGTGCTCAGGGAAAGCTGATTG
TCAGTCAAAAAGTCAAACCTGCACGTTTCCTACCACCACTTGCTGGTAGCGGTAGCGGGCAATGACTC
TTCGGGGTCTCCTGTGTCGCCTAGGCTGGCGCCGAGGTCCTCGACTGTAGAAAAGATAGTTGATGTAG
ACATACTCCAGCAAGGACAGGAACACAAAGAACAAGCACACGAGGATATAGATATCAATGGCCTTGAT
ACAGGAAATGTTGGGGAGCTTATCCCGCAGATGTGAGTCGATGGTGGTCAGGATGAGCATTGAAGTTA
AGCCTGTAAGCAACACAGTACAGACTTAGTCTCCTCTGATGGCTAACGTTCTTGGCAACCT

The following amino acid sequence <SEQ ID NO. 87> is a predicted
amino acid sequence derived from the DNA sequence of SEQ ID NO. 36:

<SEQ ID NO: 87>

GLTSMLILTTIDSHLRDKLPNISCIKAIDTYIXXXXXXXXXXXLEYVYINYLFY

The following DNA sequence Ion81 was identified in H. sapiens:

<SEQ ID NO. 37>

AAACATTCAAACTGTATCAGAGGCCAAGGCAGTTCCAGGCTGAGTGAACAGCAGTGTCAAATACTGCT
CAGGTCAGAGCTGGTGTGGCCAGTGAACTGGGAAATTTAACATCAGAGGGGGCAATCTTGACTTTCCT
CAAAGCATTCTCAGTGGAGTGGTAGGAGTAGGAGTGAGGTCCAGAAGATTTGCGGATGAGTGAGTGGC
TGAGATGGGAAAACAGCAAGTGTAGAAAACTCATACAAGTTTGGTTGTGAAGTGACAGAAAGAGTAGC
TAGAGAAGTGGAAGGATTTTCCTTAGCTGGTAGAGATCCAGGGATGCTCCATTGCTTATGAGGGGACA
GGAAAGAGGGGAGGGTTGAAGATATGGGATGAATGACAGGGAAGAAAGCATTCCCAAACACAGAGGAG
GTCCCCCAAAATGGATCCTGATACAGGTAAGTGGAAAGGTTTGTGGCAGAATGTTGAGAAACCATCCA
TTCAATGGCTTCTGTTTAGTCTCTGATATGAAAGACAAAGTCACCTGCCCAGATGGATGAAAAGATAG
TGGGATAGAAAACTGGAAAAAAAACAAAAAAGGGAAAAAGGTTTGAAATAGCCTTTGAGAAGCATGAA
GAGAGAGCTGGAGGCTTGCTGAACTCTGCTGAGAGCCAGTGGAAGCTGGAGACTG

The following amino acid sequence <SEQ ID NO. 88> is a predicted
amino acid sequence derived from the DNA sequence of SEQ ID NO. 37:

<SEQ ID NO: 88>

LSFISETKQKPLNGWFLNILPQTFPLTCIRIHFGGPPLCLGM

The following DNA sequence Ion82 was identified in H. sapiens:

<SEQ ID NO. 38>

CTTTTAAACACAGTTGGGACACTACCATTAAAGAGGAATCTTCATCACTAAAAGTAAGGTAATTTTGT
TAGAAAATGCAAATCCTAACACAAAAAATCGGATCAAAGGTAAATCACAAATAATGTTTGAGGTACAA
AGAATCTACCACTGTGGCAAAATTCAGGCCATAATAAACCACTCTTTACACAGGGGATCCAATGGGAG
ACATTTGAAAAACAGAAATACACTTTTCTTGGTGAGCAATGTTAGGTACTCCAGTTTCATCTTAACTT
TGTCTTTGGTTATGGGTCTCAAGCGTCCCTATTTCTGTAAACAAACACATAAATATTCAAAAGAGTAT
CTCTAAGTAAGTTGAGGTTTATAAAATAGAAATTTTTCTTTTTAACATACCGAGGCTTTATTTTTTAG
CTTTCTGTCTTTAGTAGCACTCTTTCCTTTTTGGTTGCTGGTAAAATAATCCAAGGTTCCATATTCCA
TCAAGGGCTGCAAAAACAAAAATGAAACAAACAGAAACAAAG

The following amino acid sequence <SEQ ID NO. 89> is a predicted
amino acid sequence derived from the DNA sequence of SEQ ID NO. 38:

<SEQ ID NO: 89>

LFLFVSFLFLQPLMEYGTLHYFTSNQKGKTATKDRKLKNKASV

The following DNA sequence Ion83 was identified in H. sapiens:

<SEQ ID NO. 39>

CTCTATGTAGCCCCAACTAAACATGTCTGTGGGCTAGATTAGCCCTTTGCCCAGCTGGCCACCAGTTG
ACCATTTCTGTAGACAAGATTCTCAGAAAGGCAACCACAGCCTCAACTTTTACAGCATTATTTTCTAC
CTAAAGAGGCATGTGCATAAATGGCAGGATGCCCAGCACACCTCATTTTACTGTGTTTCACTTTATTG
TACTTCACAATATTGCATTTTTTAACAAATGGAAGGTTTCTGGCAACCCTGTGTCAAGCAAAATCTAT
CAGTGCCATTTGTCCAACAGCATGCGCTCCCTTCCTGTCTCTGGGTCACATTTTGGTAATTTTTGCGA
CATTTCACAGTTTCTCATTATTATTATATCTGTTATGGTGATCTGTGATCAGTGATCTTTGATATTCC
TATTCTAATTGTTTCAGGGAGCCACAAACTGTCCCCATATAAGATGGAAAACTTCCAATAAATGCTGT
GTGTGTTCTGACTGCTCATCACTGATTGGCTGTTCCCTCATCTCTCTTCTTCTCCTAGGGCCTCCCTA
TTCCCTGAGAGACATCAATACTGAAATTAGGCCAATCAATAACCCTACAATGGCCTCTATGTGTTCAA
GTGAA

The following amino acid sequence <SEQ ID NO. 90> is a predicted
amino acid sequence derived from the DNA sequence of SEQ ID NO. 39:

<SEQ ID NO: 90>

LASWPPVDHFCRQDSQKGNHSLNFYRIIFYLKRHVHKWQDAQHTSFYCVSLYCTSQILHFLTNGRFLA
TLCQANLSVPFVQQHALPSCLWVTFW

The following DNA sequence Ion84 was identified in H. sapiens:

<SEQ ID NO. 40>

TCCAGCTCAGAAACTACCAGCCTTCATCAACATGCTGAGCTTAGGGGCATGGATATGTGGAGAGCAGG
ACCCTCAGTGGTGCCCTTGTGTCCCCAGTCCTGGCTGGACACTCGCCTGGCCTGGAACACTAGTGCAC
ACCCGCGGCACGCCATCACGCTGCCCTGGGAGTCTCTCTGGACACCAAGGCTCACCATCCTGGAGGCG
TAAGTGAGACAGTTCCTGCCCCAGGAATCTGCCATGCATAGCCCTCCTTTTCCCCATCTACAACCTAG
AGGCTGTCTGAGTGAATATGACCCTCCTGGCGGTCCCCGCCGGACTAGCAGTGCACCTTCACTGCCTC
GAATTCCCCTCCCACTGCCAGAACTCTGAAAGCAGCTGGGGTTGGCGTTGGGATGCCACGGTCTCCCC
CCGGCCCCGTCCAAGAAGCGGCTGGGGCTCTGGCTGTGGTGCCTTTCCCCACAGGCTCTGGGTGGACT
GGAGGGACCAGAGCCCCCAGGCTCGAGTAGACCAGGACGGCCACGTGAAGCTCAACCTGGCCCTCACC
ACGGAGACCAACTGCAACTTTGAGCTCCTCCACTTCCCCCGGGACCACAGCAACTGCAGCCTCAGCTT
CTACGCTCTCAGCAACACGGGTGCTGACAGGGCAGGGGCTCCAGGGTTGAGGAGGGGA

The following amino acid sequence <SEQ ID NO. 91> is a predicted
amino acid sequence derived from the DNA sequence of SEQ ID NO. 40:

<SEQ ID NO: 91>

RVDQDGHVKLNLALTTETNCNFELLHFPRDHSNCSLSFYALSNT

The following DNA sequence Ion85 was identified in H. sapiens:

<SEQ ID NO. 41>

AGGCCATGGCAACCTGAGCCTCTGGCCTTGCTGCAAGGGGCCGAGCCACTGCAGTCGCCATGGCTGTG
GAGGGCAGTTGCTCTGGGGAGGACAGAAGACTGATGTGCTCGGACCTCTGGGATTGCAGAGCTGCTGC
GAATGTTTGAGTCTGTCACCCTAGAGAGGCGCCCTGAGGCTACCGCTGAGCACAGAGATGGGCTGCCA
CTCGAGTGGGGGGCGCACTGGGAGAGCAGGTGCTGCCCGCCTAAGCCTGGGGTAGACTGCTCTGAACA
CAGATCTGGGAGTTCGCCTTCTGTCTGCCTTTGCCCCTTCCCCTTGCCCCGCACCCTGCCCCTGCACC
ACAGACCTGGGAGTTCCCCTCCCCCACCTTCCTCCTCCCCTCCTCAACCCTGCAGCCCCTGCCCTGTC
ACCACCCGTGTTGCTGAGAGCGTAGAAGCTGAGGCTGCAGTTGCTGTGGTCCCGGGGGAAGTGGAGGA
GCTCAAACTTGCAGTTGGTCTCCGTGGTGAGGGCCAGGTTGAGCTTCACGTGGCCGTCCTGGTCTACT
CGAGCCTGGGGGCTCTGGTCCCTCCAGTCCACCCAGAGCCTGTGGGGAAAGGCACCACAGCCAGAGCC
CCAGCCCCTTCTTGGACGGGGCCGGGGGGAGACCCTGGCA

The following amino acid sequence <SEQ ID NO. 92> is a predicted
amino acid sequence derived from the DNA sequence of SEQ ID NO. 41:

<SEQ ID NO: 92>

RVDQDGHVKLNLALTTETNCNFELLHFPRDHSNCSLSFYALSNT

The following DNA sequence Ion86 was identified in H. sapiens:

<SEQ ID NO. 42>

AATTATAGAAAATCCAAATATCCTGGCTGGGGTGAGAGTCTGTAAGCTAGCCAGAGAAAACAGCTAAG
GCTAAGAAAATAAAATATAGGAGAAAATTCTAGAAAATCCAGATATCCTGGCTGGGGTGAGAGTCTGT
AAGCTAGCCAGAGAAAAGAGCTGAGGCGAAGACAATAAAATATAGGAGAAAATTCTAGAAAAATGAAA
ATTGGTTTATTGTCCCAGATCTGTACCCTTCTCCCCCTCTGATTGTTCACTTGATTTTAGATGGTGAA
TGACAAATATTGGTGAAGAAAATCATTCCATGAAACACTGGTAACCATTTGTCCGAAACGCCTTCATG
GCAGCACTGCCGTGGCTCAGTACATTGCACCTGCACTTCCAAAGTGAAGGTGACTGTTACCTGAAACC
CATGTGCCTGGCACACATGACCAGCCTTGGACACAAGAGGCCTTTGATCAGAAACTGGGAGGCACTCC
CACATTCCCACAATGAAATTCCGTGGGTGCCTGTACCCTGAGTTCATCCAACACATGGTTACTGATCA
TGTAGGGTGTACCAGGCTATGTCAGACGTTAGAGACACCATGAAGAGCAAACAGTTAGCTTATGGCGA
GTGCCTAACGCACACCTGCCATTTACATCTTTGTCCTCATGATTCTTCCCACTGAACCAATGGCACTG

The following amino acid sequence <SEQ ID NOS. 93> is a predicted
amino acid sequence derived from the DNA sequence of SEQ ID NO. 42:

<SEQ ID NO: 93>

LEFSPIFYCLRLSSFLWLAYRLSPQPGYLDFLEFSPIFYFLSLSCFLWLAYRLSPQPGY

The following DNA sequence Ion87 was identified in H. sapiens:

<SEQ ID NO. 43>

ATGTCTCTTTGTTTAATTAGTTTTGGGTGGCTCAATTTTTAGGACTATTGTTCCTGTTTTTCTTTCCT
CAGTTTTAATTGCCAATTTAAGCTCTGGACAAAATCTGAAAATTTACAACTGGAATTTTACAAGAAGG
CCTCGTATTATAAAGTTTGTTCCTTGGTTTGTGAGACTTGGGTTGTGGACAGTTTGAATAACGTTTTC
ATAGAAAAGCATCAGTGAAAGAAAGAAAATAAAATATATTTTAAACTAACTTTCCTCCTTCCAATAAA
ACTTCTAAAAGTCAATACATATGACTTTTTCAAAAACATAAAAAAAAATGCCAGATATAGGGCTCTTC
ACCCAAAGATTAAAATAAGTTTTTTTTAAAACAAACAAACAAACAAACAAAAAGAACATATGGCTGAA
ATAAAAGTGCCTCTTGGTAGAATATGCAATGAAAGTGTAGGTTGGGTCCAGAGAAACACTTGTGTGCA
GACATCAATTCTCAGGAGACAATGAGGAGTGAAGCAAACAAGATTGAATGGCGGAAAGTTGAAGGGTG
ATACTGTTGAAATAG

The following amino acid sequence <SEQ ID NO. 94> is a predicted
amino acid sequence derived from the DNA sequence of SEQ ID NO. 43:

<SEQ ID NO: 94>

FNFPPFNLVCFTPHCLLRIDVCTQLFLWTQPTLSLHIL

The following DNA sequence Ion88 was identified in H. sapiens:

<SEQ ID NO. 44>

TCTCCTTCATAGATTACTCTTTTCATTACCCTTGTGCCATATAACATCTTAGCTGTGTGACACCAGGG
AGAAAGGTGTTGGTCACCTACCCTTGGCAGTAGGAAGTCTTTCAGATCTGATATTAATTGTGTATTCA
AATGTCAAGGTATCCTAGTACAGAAAATATCAGTGGGTTATTCTGATAAGGAAAATACTATTTGCTAA
TTTTAGAAAAGAGAATATGCTAAAAGTTACACCTCAGAGGGAATATCATTTGATATGGTGAACAGGAA
ACCCAAGAAGTTGTGAATTCCATTCAAAAGATGAAACTGCTTAGAAGATAATGTAAGGTTCTCACCCA
ACATGAGCACTGCACTCAAGGCCATTTCTAGGATGAAAGGGTGGGATGATTATCTATTATTCCAGCCA
TGAATTATTTCTGTGGCCTCCAGAAGATGCAACTGAATTGTAGCTATGTGTCCAGAATCGGTTCCTTC
TGGTGGCTTCTTGGTCTCGCTGACTTCAAGAATGAAGCCGTCGACCCTCACGGTGAGTGTTGCAGTTC
TTAAAGATGGTGTGTCTGGACTTTGTTCCTTCAGATATTCAGATGTGTCCCGGAGTTTCTTTCTTCTG
GTGGGTTTTGTGGTCTTACTGACTTCAGGAGTGAAGC

The following amino acid sequence <SEQ ID NO. 95> is a predicted
amino acid sequence derived from the DNA sequence of SEQ ID NO. 44:

<SEQ ID NO: 95>

ASRRCNIVAMCPESVPSGGFLVSLTSRMKPWTLTVSVAVLKDGVSG

The following DNA sequence Ion89 was identified in H. sapiens:

<SEQ ID NO. 45>

AGCAGTCGCATGATAGCTTCATTCCTTGGAGTCTATTGTGTGTGTTTGGGGCCCCGTAAAATATTAGA
AAGCGATGGAAATTTTAGGGCTCCGTATAATATTGTATTTACATAACCACTCAGCTCTCAACTACTCT
CAAAGAGTACCTACTGAAGATCATGTCTTCAACTTGCTAAGGCTGATCTGGGTATTAGCCAACTCTCT
GAGTTGAACGAAACAGATGTAACCAGGTCATCTCATGAAATGGAGCTCTATTGTTCAGTAGATGAGGT
AGTAAGTGGAGCAGACACTGCTGTTTGCCTTCTCCCTGGCTAACAGAGGACTGACATTGACTGGATTA
AAGGATAGAGCTACCCTGTACTTCAGGCGGCTGCATTCCTCCCTGCCGGCACCAGTGATTGATTTAGG
AATGGGTAGAGGGTGCAATTCTGACCAATGAGACGTGGGAGAAGCTTGCTGGGGAGTTGGTGGGGTAT
TTTCCTTTTGCTTTAAAAGGGGCAAAGGAAAGGTACATTCCCTTTTTTTTTCCTTTTTCATCTCTGGA
TGTCATTGCCTGGAACTTTTGCAGGCTTCTGATACCATGAG

The following amino acid sequence <SEQ ID NO. 96> is a predicted
amino acid sequence derived from the DNA sequence of SEQ ID NO. 45:

<SEQ ID NO: 96>

GAILTNETWEKLAGELVGYFPFALKGAKERYIPFFFPFSSLDV

The following DNA sequence Ion90 was identified in H. sapiens:

<SEQ ID NO. 46>

CCCAGCAGAACATAAGGTTGTGGCTGGGACATGAATGCACCCCAGGGAGCACTGAACTGCTCTGAGCT
GCCGACTAGGGCCATAGGCTAGCTATGTGGGCCCATATTGAGGTAGGGGCTGAGCAGTCCCAGCGGCA
CCGCCCAGGCTGCCTGCTCTGGGGTCCCTGCAAAAGCCGCCGCTGAGCCCACGGACTTCCGGGTCGTA
AGCACGTGCGGCCTGAACATCTGCTTGGCTGGGTCAGCTCCTATGACAATGCCCGGGCGATCGTGCCC
TCCAGCGCTGCCTGCATGCCGAGGAGGAAGCGAGTCCCCACGTGAATAATCGGGCTCCGCCGGCTCAC
AGCGGATGTCAGAAGGTCAGGTCGCTCTGCTCCTTTCGCCTCCGTTTTTCTTCCTCATGGAAACTTTC
TTCAGCTGCAGAAAAAGCTGGTCCTTTTCTTTCTGCCTGGCCCACAGCTTCTCCTGCAAAGTCAAATT
TTGTTCCTTCGGTCTCCTCTGGTGACATTCTCTCTTCCATCTCCTTTCTCTTCCTTCGTCTGCTCTTC
CTCCATCTTCTCGCCATCACCTTATCCGCCGCCTCCTCTTCCCTCTCGCCCCGCAGCCTGCGCTCCCG
CCGGGGGCGCTCCGGACACACTGTCTGCGC

The following amino acid sequence <SEQ ID NO. 97> is a predicted
amino acid sequence derived from the DNA sequence of SEQ ID NO. 46:

<SEQ ID NO: 97>

KRECHQRRPKEQILTLQEKLWARQKEKDQLFLQLKKVSMRKKNGGERSRATPSDIRCEPAEPDYSRGD
SLPPRHAGSAGGHDRPGIVIAADPAKQMFRPHVLTTRKSVGSAAAFAGTPEQAAWAVPLGLLSPYLNM
GPHSPMALVGSSEQFSAPWGAFMSQPQP

The following DNA sequence Ion91 was identified in H. sapiens:

<SEQ ID NO. 47>

CTTTTTCTGCAGCTGAAGAAGTTTCCATGAGGAAGAAAAACGGAGGCGAAAGGAGCAGAGCGACCTGA
CCTTCTGACATCCGCTGTGAGCCGGCGGAGCCCGATTATTCACGTGGGGACTCGCTTCCTCCTCGGCA
TGCAGGCAGCGCTGGAGGGCACGATCGCGCGGGCATTGTCATAGCAGCTGACCCAGCCAAGCAGATGT
TCAGGCCCCACGTGCTTACGACCCGGAAGTCCGTGCGCTCAGCGGCGGCTTTTGCAGGGACCCCAGAG
CAGGCAGCCTGGGCGGTGCCGCTGGGACTGCTCAGCCCCTACCTCAATATGGGCCCACATAGCTAGCC
TATGGCCCTAGTCGGCAGCTCAGAGCAGTTCAGTGCTCCCTGGGGTGCATTCATGTCCCAGCCACAAC
CTTATGTTCTGCTGGGCCACTTTCAGCACACCCAGACAGGGTTCCTCTTCTGGTGCTGCTCTGTCTTT
GAAACCGCAGATAGACCATGCTAACCAGCACACAGGTTTCCCTGGTCCATCCTCCCTGACCCCCATGC
ATGCCCAGGCTCTGCATCCAGGCCCTAGACTCCTTGCCTAATCCCAGCTCCCCGCGAAGATGCAGCCA
GCAGGAACGTCTAGGTTTTGCAGCTACCAACCAACCAGGCCCTCA

The following amino acid sequence <SEQ ID NO. 98> is a predicted
amino acid sequence derived from the DNA sequence of SEQ ID NO. 47:

<SEQ ID NO: 98>

GSAGGHDRPGIVIAADPAKQMFRPHVLTTRKSVGSAAAFAGTPEQAAWAVPLGLLSPYLNNGPHSPMALVG
SSEQFSAPWGAPMSQPQPYVLLGHFQHTQTGFL

The following DNA sequence Ion92 was identified in H. sapiens:

<SEQ ID NO. 48>

TCTGAAGCTGCCGTGTATGAACATACATCTACACATACACACACACACACACACACACACACACACAC
ACACACACACACACACACACACACCCCCCGTAGTGATAAAACTATGTAAATGATATTTCCATAATTAA
TACGTTTATATTATGTTACTTTTAATGGATGAATATGTATCGAAGCCCCATTTCATTTACATACACGT
GTATGTATATCCTTCCTCCCTTCCTTCATTCATTATTTATTAATAATTTTCGTTTATTTATTTTCTTT
TCTTTTGGGGCCGGCCCGCCTGGTCTTCTGTCTCTGCGCTCTGCTGACCTCAGCCTCCCAAATAGCTG
GGACTACAGGGGATCTCTTAAGCCCGGGAGGGAGAGGTTAACGTGGGCTGTGATCGCACACTTCCACT
CCACCTTACGTGGGCTGCGGTGGGGTGGGGTGGGGTGG

The following amino acid sequence <SEQ ID NO. 99> is a predicted
amino acid sequence derived from the DNA sequence of SEQ ID NO. 48:

<SEQ ID NO: 99>

CIEAPFHLHTRVCISFLPSFIHYLLIIFVYLFSFLLGPARLVFCLCALVTSASQIAGTTGDL

The following DNA sequence Ion93 was identified in H. sapiens:

<SEQ ID NO. 49>

TCTATAGCTCCCACCCTATTCACAGAAGCCTGGTGGATATCTTCTGACCGTAGCACTTTATAGACAAC
CCAGTAGAAGATATTGAAGATGAGGAAAGTGAAAGGGAAGACAGCCCGGGAGATGGTGTCAATTCTCT
TGGCTCAGTCCACGTAGAGTTTCCGCGTGGTTTCTCCTTCCCTTAGAAGAGGGGCTGGAGGTTGGGGA
CTATAAATGCCAGAACCTTCCATTGGACCTCCATCTCTTGCCTGCAGGCAGTGGCCCAAGCCATAGCC
ACGGAAATAGAAACGACTTTCTTGGATGATATCTTCCTCCTGGAATTACAAGGAAGAAACGGCAGAAT
TTGAGGTCAAAGCTCAAAGGCAGAGGGATAGAGAACAGACCACCCATCAATATCTCATAGGGAATGTT
ATGCAGACAAGGTGCCTTGGGTACACAGGCCCATTGCATGCTTTTTATGGTCACAACACTACTCATGA
GATAGATGTATGCTAAGCAGCTCTACGTGCTATATATAGTGTATGTCATGATTCCATGGCAGATAGCC
TCTAAGCTAC

The following amino acid sequence <SEQ ID NO. 100> is a predicted
amino acid sequence derived from the DNA sequence of SEQ ID NO. 49:

<SEQ ID NO: 100>

QEEDIIQESRFYFRGYGLGHCLQARDGGPMEGSGIYSPQPPAPLLREGETTRKLYVDAKRIDTISRAV
FPFTFLIFNIFYWVVYKVLRSEDIHQ

The following DNA sequence Ion94 was identified in H. sapiens:

<SEQ ID NO. 50>

TCATTAATTTATTACTAAGCACTAGTGGAAATCTAACTTTATTTACCCCCATCAACTTGGCTTGTGTT
ACCAGAACAAGAAGGCAACCAAACATGAAATGCTTTGGGAAATGACCCACTAGACTGAACGTCCAAAT
CACTTTTGCTGTTACATACTGTATGACAGCGGTTCTCAAACCTCTGTGTGCAGAACACCCCTGAGAAC
TTGTTAAAATAACGGTTCCTGAGCCCCAGCCCAGAGCGTATGGTTCAGTAGTTTGGGGCTGAGGTTGG
AGAATTTGCATTTGTAGTAAGTTCCCAGGTGATACTGCTGCTGCCACTGGTCCTGGACTACACTTTGA
GGAGCCTGCTGAACACAGCACCTCAGCCTCTACTTGAAGGACAAACTAGCTTCTTACTGGATTCAGTG
GCAAGATTAAGCCCACTGGTTCTCAAACACAATCCCCTTGGGAACACCAGTGCTCTACCAC

The following amino acid sequence <SEQ ID NO. 101> is a predicted
amino acid sequence derived from the DNA sequence of SEQ ID NO. 50:

<SEQ ID NO: 101>

ENRCHTVCNSKSDLDVQSSGSFPKAFHVWLPSCSGNTSQVDGG

The following DNA sequence Ion95 was identified in H. sapiens:

<SEQ ID NO. 51>

TCAGCATTTTGTTGCCAAGTTTTCTGAGACCTCTGGCCATTAAGCCTTCACTGGGGGTGTGGTCTGTC
TCTGAAGTCTACTCCCATTGCAAATGGATTTTGACGGTAATGGTCAACACGCCTGGGCAAAGAATGGG
TCATGCCCATTCTTACTGGAAAGATTTGGAACATTTCCCTGTAAATTGTATATTATTTGGATTTATTT
CTCTAACTGAATGGACGTTTTTCTATATGTTGCCAAATCTTCCAGTAATCCTTCTCATTCAGTCTAAT
TAAGGAGATTAAAAGTGACAGCATTTTTCTTGTTGAATTAATGATGGGTTTTTACATTTTCACTTTTC
AAAAAATATAATCACCACTGTGTTTTGCAGAAACAATAGTATGATAAAATCAAGGAGAAATACAACTA
GAGAAGAGGCAAAAAAATCTCAATATTATGATTATAA

The following amino acid sequence <SEQ ID NO. 102> is a predicted
amino acid sequence derived from the DNA sequence of SEQ ID NO. 51:

<SEQ ID NO: 102>

AIKPSLGVWSVSEVYSHCKWILTVMVNTPGQRMGHAHSYWKDLEHFPVNCILFGFISLTEWTFFYMLP
NLP

The following DNA sequence Ion31d6 was identified in H. sapiens:

<SEQ ID NO. 103>

GGAATTCCCGGGATGCTCACCAACATCAGCGTCCCCACCCAAGTCAACATCTCCTTCGCGATGTCTGC
CATCCTAGATGTGAATGAACAGCTGCACCTCTTGTCATCATTCCTGTGGCTGGAAATGGTTTGGGATA
ACCCATTTATCAGCTGGAACCCAGAGGAATGTGAGGGCATCACGAAGATGAGTATGGCAGCCAAGAAC
CTCTGGCTCCCAGACATTTTCATCATTGAACTCATGGATGTGGATAAGACCCCAAAAGGCCTCACAGC
ATATGTAAGTAATGAAGGTCGCATCAGGTATAAGAAACCCATGAAGGTGGACAGTATCTGTAACCTGG
ACATCTTCTACTTCCCCTTCGACCAGCAGAACTGCACACTCACCTTCAGCTCATTCCTCTACACAGTG
GACAGCATGTTGCTGGACATGGAGAAAGAAGTGTGGGAAATAACAGACGCATCCCGGAACATCCTTCA
GACCCATGGAGAATGGGAGCTCCTGGGCCTCAGCAAGGCCACCGCAAAGTTGTCCAGGGGAGGCAACC
TGTATGATCAGATCGTGTTCTATGTGGCCATCAGGCGCAGGCCCAGCCTCTATGTCATAAACCTTCTC
GTGCCCAGTGGCTTTCTGGTTGCCATCGATGCCCTCAGCTTCTACCTGCCAGTGAAAAGTGGGAATCG
TGTCCCATTCAAGATAACGCTCCTGCTGGGCTACAACGTCTTCCTGCTCATGATGAGTGACTTGCTCC
CCACCAGTGGCACCCCCCTCATCGGTGTCTACTTCGCCCTGTGCCTGTCCCTGATGGTGGGCAGCCTG
CTGGAGACCATCTTCATCACCCACCTGCTGCACGTGGCCACCACCCAGCCCCCACCCCTGCCTCGGTG
GCTCCACTCCCTGCTGCTCCACTGCAACAGCCCGGGGAGATGCTGTCCCACTGCGCCCCAGAAGGAAA
ATAAGGGCCCGGGTCTCACCCCCACCCACCTGCCCGGTGAGGTGTGA

The following amino acid sequence <SEQ ID NO. 105> is a predicted
amino acid sequence derived from the DNA sequence of SEQ ID NO. 103:

<SEQ ID NO: 105>

GIPGMVTNISVPTQVNISFAMSAILDVNEQLHLLSSFLWLEMVWDNPFISWNPEECEGITKMSMAAKN
LWLPDIFIIELMDVDKTPKGLTAYVSNEGRIRYKKPMKVDSICNLDIFYFPFDQQNCTLTFSSFLYTV
DSMLLDMEKEVWEITDASRNILQTHGEWELLGLSKATAKLSRGGNLYDQIVFYVAIRRRPSLYVINLL
VPSGFLVAIDALSFYLPVKSGNRVPFKITLLLGYNVFLLMNSDLLPTSGTPLIGVYFALCLSLMVCSL
LETIFITHLLHVATTQPPPLPRWLHSLLLHCNSPGRCCPTAPQKENKGPGLTPTHLPGEV

The following DNA sequence Ion31c4 was identified in H. sapiens:

<SEQ ID NO. 104>

TGGTACCGGTCCGGAATTCCCGGGATCACGCCCTGCCTTGGGGCCCCTCTCATATAGGGAGCACAGGG
TTGCTCTCCTTCATCTCACACATTCGATGTCCACTACAGGAAGGGGCGTTACTTTCACCATCAATTGC
TCAGGGTTTGGCCAGCACGGGGCGGATCCCACTGCTCTGAATTCAGTGTTTAATAGAAAGCCCTTCCG
TCCGGTCACCAACATCAGCGTCCCCACCCAAGTCAACATCTCCTTCGCGATGTCTGCCATCCTAGATG
TGAATGAACAGCTGCACCTCTTGTCATCATTCCTGTGGCTGGAAATGGTTTGGGATAACCCATTTATC
AGCTGGAACCCAGAGGAATGTGAGGGCATCACGAAGATGAGTATGGCAGCCAAGAACCTGTGGCTCCC
AGACATTTTCATCATTGAACTCATGGATGTGGATAAGACCCCAAAAGGCCTCACAGCATATGTAAGTA
ATGAAGGTCGCATCAGGTATAAGAAACCCATGAAGGTGGACAGTATCTGTAACCTGGACATCTTCTAC
TTCCCCTTCGACCAGCAGAACTGCACACTCACCTTCAGCTCATTCCTCTACACAGTGGACAGCATGTT
GCTGGACATGGAGAAAGAAGTGTGGGAAATAACAGACCCATCCCGGAACATCCTTCAGACCCATGGAG
AATGGGAGCTCCTGGGCCTCAGCAAGGCCACCGCAAAGTTGTCCAGGGGAGGCAACCTGTATGATCAG
ATCGTGTTCTATGTGGCCATCAGGCGCAGGCCCAGCCTCTATGTCATAAACCTTCTCGTGCCCAGTGG
CTTTCTGGTTGCCATCGATGCCCTCAGCTTCTACCTGCCAGTGAAAAGTGGGAATCGTCTCCCATTCA
AGATAACGCTCCTGCTGGGCTACAACGTCTTCCTGCTCATGATGAGTGACTTGCTCCCCACCAGTGGC
ACCCCCCTCATCGGTGTCTACTTCGCCCTGTGCCTGTCCCTGATGGTGGGCAGCCTGCTGGAGACCAT
CTTCATCACCCACCTGCTGCACGTGGCCACCACGCAGCCCCCACCCCTGCCTCGGTGGCTCCACTCCC
TCCTGCTCCACTGCAACAGCCCGGGGAGATGCTGTCCCACTGCGCCCCAGAAGGAAAATAAGGGCCCG
GGTCTCACCCCCACCCACCTGCCCGGTGTGAAGGAGCCAGAGGTATCAGCAGGGCAGATGCCGGGCCC
TGCGGAGGCAGAGCTGACAGGGGGCTCAGAATGGACAAGGGCCCAGCGGGAACACGAGGCCCAGAAGC
AGCACTCAGTGGAGCTGTGGTTGCAGTTCAGCCACGCGATGGACGCCATGCTCTTCCGCCTCTACCTG
CTCTTCATGGCCTCCTCTATCATCACCGTCATATGCCTCTGGAACACCTAGGCAGGTGCTCACCTGCC
AACTTCAGTCTGGAGCTTCTCTTGCCTCCAGGGACTGGCCAGGTCTCCCCCCTTTCCTGACTACCAAC
TATCATATCCCCAAAGATGACTGAGTCTCTCCTGTATTCCATGTATCCCAATCCGGTCCTGCTGATCA
ATTCCAATCCCAGACATTTCTCCCTGTTCCTGCATTTTGTTGGCTTCCTTCAGTCCTACCATATGGTT
CTAGGTCCCTCTTACGTCATCTGCATAGCAGACTATACCTCTTCTGCCCGCTGACTTGCCCAATAAAT
AATTCTGCAGAGAAAAAAAAAAAAAAAAAAAAAAAAAAAAAAAAAAAAAAAAAAAAAAAAAAAAAAGG
GCGGCCGCTCT

The following amino acid sequence <SEQ ID NO. 106> is a predicted
amino acid sequence derived from the DNA sequence of SEQ ID NO. 104:

<SEQ ID NO: 104>

GTGPEFPGSRPALGPLSYREHRVALLHLTHSMSTTGRGVTFTINCSGFGQHGADPTALNSVFNRKPFR
PVTNISVPTQVNISFAMSAILDVNEQLHLLSSFLWLEMVWDNPFISWNPEECEGITKMSMAAKNLWLP
DIFIIELMDVDKTPKGLTAYVSNEGRIRYKKPMKVDSICNLDIFYFPFDQQNCTLTFSSFLYTVDSML
LDMEKEVWEITDASRNILQTHGEWELLGLSKATAKLSRGGNLYDQIVFYVAIRRRPSLYVINLLVPSC
FLVAIDALSFYLPVKSGNRVPFKITLLLGYNVFLLMMSDLLPTSGTPLIGVYFALCLSLMVGSLLETI
FITHLLHVATTQPPPLPRWLHSLLLHCNSPGRCCPTAPQKENKGPCLTPTHLPGVKEPEVSAGQMPGP
AEAELTGGSEWTRAQREHEAQKQHSVELWLQFSHAMDAMLFRLYLLFMASSIITVICLWNTAGAHLPT
SVWSFSCLQGLARSPPFPEYQLSYPQRLSLCCIPCIPIRSCSIPIPDISPCSCILLASFSFTIWFVPL
TSSAQTIPLLPADLPNKFCREKKKKKKKKKKKKKKKKKRAAA

The following DNA sequence Ion52 was identified in H. sapiens:

<SEQ ID NO. 107>

CTGGAAAGCTCCATCGCGTGGCTGAACTGCAACCACAGCTCCACTGAGTGCTGCTTCTGGGCCTCGTG
TTCCCCCTGGGCCCTTGTCCATTCTGAGCCCCCTGTCAGCTCTGCCTCCGCAGGGCCCGGCATCTGCC
CTGCTGATACCTCTGGCTCCTTCACACCTACAGAAAGACAGAGACTCAGCCATCGGCTGCAAATGTCA
CCTGTGGAGGGAGGGAGACAGGGAAGAAGGCAGGAGCAGAGAAGTGGAGGTGGGGGAAGAGGAATGTG
ACTTCCCTCACCGGGCAGGTGGGTGGGGCGTGAGACCCGGGCCCTTATTTTCCTTCTGGGGCGCAGTG
GGACAGCATCTCCCCGGGCTGTTGCAGTGCAGCAGCAGGGACTGGAGCCACCGAGGCAGGGGTGGGGG
CTGGGTGGTGGCCACGTGCAGCAGGTGGGTGATGAAGATGGTCTCCAGCACGCTGCCCACCATCAGGG
ACAGGCACA

The following amino acid sequence <SEQ ID NO. 109> is a predicted
amino acid sequence derived from the DNA sequence of SEQ ID NO. 107

<SEQ ID NO: 109>

CLSLMVGSLLETIFITHLLHVATTQPPPLPRWLHSLLL

The following DNA sequence Ion 111 was identified in H. sapiens:

<SEQ ID NO. 108>

CCCAGCACTTTGGGAGGCCAAGGTGGGTGGATCACTTCAGTTCAGGAGTTTGAGACCAGCCTGGGCAA
CATGGTGAAACCTCATCTCTTAAAAAAAAAAAAAAAAAAAAAATTAGCCAGGCCTGGTGGTGCGCCTG
TAGTCCCAGCTACTTGGGAGGCTGAGGCTGAGACAGGAGGATCATTTGAGCCCAGGACATGGAAGTTG
CAGTGACCTGAGAGCATGCCACTCTACTCCAGCCTGGGTGACAGAGCAAGATCCTGTCTCAAAAAAAA
AAAAAAAAAAAAAGGAGAGAGAGAAACTGCGGCCCCTGCCTCTTGCGTTATCTCTCCTCCAGCATGGA
TGTGGATAAAACCCCAAAAGGCCTCACAGCATATGTAAGTAATGAAGGTCGCATCAGGTATAAAAAAC
CCATGAAGGGGGACAGTATCTGTAACCTGGACATCTTCTACTTCCCCTTCGACCAGCAAAACTGCACA
CTCACCTTCAGCTCATTCCTCTACACAGGTAAGTTGCAGTGAGGTCTCAGGGATGGGGTGAATGAGAG
CAACCAACAAATTTAAAGAAACTATGAGTAAATGGTGACC

The following amino acid sequence <SEQ ID NO. 110> is a predicted
amino acid sequence derived from the DNA sequence of SEQ ID NO.108:

<SEQ ID NO: 110>

LSSSMDVDKTPKGLTAYVSNEGRIRYKKPMKGDSICNLDIFYFPFDQQNCTLTFSSFLYT

### EXAMPLES

### Example 1

Identication of Ion Channel Sequences in GenBank/EMBL

A brief description of the searching mechanism follows. The BLAST algorithm, Basic Local Alignment Search Tool, is suitable for determining sequence similarity (Altschul et al ., J. Mol. Biol., 1900, 215, 403-410, which is incorporated herin by reference in its entirety). Software for performing BLAST analyses is publicly available through the National Center for Biotechnology Information (http://www.ncbi.nlm.nih.gov/). This algorithm involves first identifying high scoring sequence pair (HSPs) by identifying short words of length “W” in the query sequence that either match or satisfy some positive valued threshold score “T” when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The BLAST algorithm parameters W, T and X determine the sensitivity and speed of the alignment. The BLAST program uses as defaults a word length (W) of 11, the BLOSUM62 scoring matrix (see Henikoff et al., Proc. Natl. Acad. Sci. USA, 1992, 89,10915-19, which is incorporated herein by reference in its entirety) alignments (B) of 50, expectation (E) of 10, M=5, N=4, and a comparison of both strands.

The BLAST algorithm (Karlin et al., Proc. Natl. Acad. Sci. USA, 1993, 90, 5873-5787, which is incorporated herein by reference in its entirety) and Gapped BLAST (Altschul et al., Nuc. Acids Res., 1997, 25, 3389-3402, which is incorporated herein by reference in its entirety) perform a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a nucleic acid is considered similar to an ion channel gene or cDNA if the smallest sum probability in comparison of the test nucleic acid to an ion channel nucleic acid is less than about 1, preferably less than about 0.1, more preferably less than about 0.01, and most preferably less than about 0.001.

The Celera database was searched with the NCBI program BLAST (Altschul et al., Nuc. Acids Res., 1997, 25, 3389, which is incorporated herein by reference in its entirety), using the known protein sequences of ion channels from the SWISSPROT database as query sequences to find patterns suggestive of novel ion channels. Specifically, one of the BLAST programs TBLASTN was used to compare protein sequences to the DNA database dynamically translated in six reading frames. Alternatively, a second search strategy was developed using a hidden Markov model (HMM)(Krogh, A., Brown, B., Mian, I S., Sjolander, K and D.Haussler, Hidden Markov models in computational biology: applications to protein modeling. J Mol Biol 1994, 235;1501-1531) ) to query that nucleotide database translated in six reading frames. HMMs, as used herein, describe the probability distribution of conserved sequence when compared to a related protein family. Because of this different search algorithm, the use of HMMs may yield different and possibly more relevant results than are generated by the BLAST search. Positive hits were further analyzed with the program BLASTX against the non-redundant protein and nucleotide databases maintained at NCBI to determine which hits were most likely to encode novel ion channels, using the standard (default) parameters. This search strategy, together with the insight of the inventors, identified SEQ ID NO:1 to SEQ ID NO:51 and SEQ ID NOS:103, 104, 107, and 108, as candidate sequences.

### Example 2

Detection of Open Reading Frames and Prediction of the Primary Transcript for Ion Channels

The predictions of the primary transcript and mature mRNA were made manually. Consensus sequences found in textbooks (i.e., Lodish, H. et al. Molecular Cell Biology, 1997, ISBN: 0-7167-2380-8) and regions of similarity to known ion channels were used to discover the primary transcripts of the ion channel polypeptides.

### Example 3

Cloning of Ion Channel cDNA

To isolate cDNA clones encoding full length ion channel proteins, DNA fragments corresponding to a portion of SEQ ID NO:1 to SEQ ID NO:51, and SEQ ID NOS:103, 104, 107, and 108, or complementary nucleotide sequence thereof, can be used as probes for hybridization screening of a phage, phagemid, or plasmid cDNA library. The DNA fragments are amplified by PCR. The PCR reaction mixture of 50 μl contains polymerase mixture (0.2 mM dNTPs, 1×PCR Buffer and 0.75 μl Expand High Fidelity Polymerase (Roche Biochemicals)), 100 ng to 1 μg of human cDNA, and 50 pmoles of forward primer and 50 pmoles of reverse primer. Primers may be readily designed by those of skill in the art based on the nucleotide sequences provided herein. Amplification is performed in an Applied Biosystems PE2400 thermocycler using for example, the following program: 95° C. for 15 seconds, 52° C. for 30 seconds and 72° C. for 90 seconds; repeated for 25 cycles. The actual PCR conditions will depend, for example on the physical characteristics of the oligonucleotide primers and the length of the PCR product. The amplified product can be separated from the plasmid by agarose gel electrophoresis, and purified by Qiaquick™ gel extraction kit (Qiagen).

A lambda phage library containing cDNAs cloned into lambda ZAPII phage-vector is plated with E. coli XL-1 blue host, on 15 cm LB-agar plates at a density of 50,000 pfu per plate, and grown overnight at 37° C.; (plated as described by Sambrook et al., supra). Phage plaques are transferred to nylon membranes (Amersham Hybond NJ), denatured for 2 minutes in denaturation solution (0.5 M NaOH, 1.5 M NaCl), renatured for 5 minutes in renaturation solution (1 M Tris pH 7.5, 1.5 M NaCl), and washed briefly in 2×SSC (20×SSC: 3 M NaCl, 0.3 M Na-citrate). Filter membranes are dried and incubated at 80° C. for 120 minutes to cross-link the phage DNA to the membranes.

The membranes are hybridized with a DNA probe prepared as described above. A DNA fragment (25 ng) is labeled with α-³²P-dCTP (NEN) using Rediprime™ random priming (Amersham Pharmacia Biotech), according to manufacturers instructions. Labeled DNA is separated from unincorporated nucleotides by S200 spin columns (Amersham Pharmacia Biotech), denatured at 95° C. for 5 minutes and kept on ice. The DNA-containing membranes (above) are pre-hybridized in 50 ml ExpressHyb™ (Clontech) solution at 68° C. for 90 minutes. Subsequently, the labeled DNA probe is added to the hybridization solution, and the probe is left to hybridize to the membranes at 68° C. for 70 minutes. The membranes are washed five times in 2×SSC, 0.1% SDS at 42° C. for 5 minutes each, and finally washed 30 minutes in 0.1×SSC, 0.2% SDS. Filters are exposed to Kodak XAR film (Eastman Kodak Company, Rochester, N.Y., USA) with an intensifying screen at −80° C. for 16 hours. One positive colony is isolated from the plates, and replated with about 1000 pfu on a 15 cm LB plate. Plating, plaque lift to filters, and hybridization are performed as described above. About four positive phage plaques may be isolated form this secondary screening.

cDNA containing plasmids (pBluescript SK-) are rescued from the isolated phages by in vivo excision by culturing XL-1 blue cells co-infected with the isolated phages and with the Excision helper phage, as described by the manufacturer (Stratagene). XL-blue cells containing the plasmids are plated on LB plates and grown at 37° C. for 16 hours. Colonies (18) from each plate are re-plated on LB plates and grown. One colony from each plate is stricken onto a nylon filter in an ordered array, and the filter is placed on a LB plate to raise the colonies. The filter is hybridized with a labeled probe as described above. About three positive colonies are selected and grown up in LB medium. Plasmid DNA is isolated from the three clones by Qiagen Midi Kit (Qiagen) according to the manufacturer's instructions. The size of the insert is determined by digesting the plasmid with the restriction enzymes NotI and SalI, which establishes an insert size.

The clones are sequenced directly using an ABI377 fluorescence-based sequencer (Perkin-Elmer/Applied Biosystems Division, PE/ABD, Foster City, Calif.) and the ABI PRISM™ Ready Dye-Deoxy Terminator kit with Taq FSTM polymerase. Each ABI cycle sequencing reaction contains about 0.5 μg of plasmid DNA. Cycle-sequencing is performed using an initial denaturation at 98° C. for 1 minute, followed by 50 cycles using the following parameters: 98° C. for 30 seconds, annealing at 50° C. for 30 seconds, and extension at 60° C. for 4 minutes. Temperature cycles and times are controlled by a Perkin-Elmer 9600 thermocycler. Extension products are purified using Centriflex™ gel filtration cartridges (Advanced Genetic Technologies Corp., Gaithersburg, Md.). Each reaction product is loaded by pipette onto the column, which is centrifuged in a swinging bucket centrifuge (Sorvall model RT6000B tabletop centrifuge) at 1500×g for 4 minutes at room temperature. Column-purified samples are dried under vacuum for about 40 minutes and dissolved in 5 μl of DNA loading solution (83% deionized formamide, 8.3 mM EDTA, and 1.6 mg/ml Blue Dextran). The samples are heated to 90° C. for three minutes and loaded into the gel sample wells for sequence analysis using the ABI377 sequencer. Sequence analysis is performed by importing ABI377 files into the Sequencer program (Gene Codes, Ann Arbor, Mich.). Generally, sequence reads of up to about 700 bp are obtained. Potential sequencing errors are minimized by obtaining sequence information from both DNA strands and by re-sequencing difficult areas using primers annealing at different locations until all sequencing ambiguities are removed.

Ion31

The GENETRAPPERR (patent pending) cDNA Positive Selection System was used to isolate potentially full-length clones using the sequence of ion31 set forth herein as SEQ ID NO:1. The GENETRAPPER System isolates cDNA clones from DNA prepared from a cDNA library (representing 10¹² DNA molecules). In this system, an oligonucleotide complementary to a segment of the target cDNA, is biotinylated at the 3′-end with biotin-14-dCTP using terminal deoxynucleotidyl transferase (TdT). Simultaneously, a complex population of ds phagemid DNA containing cDNA inserts (10⁶ to 10⁷ individual members) is converted to ssDNA using Gene II (phage F1 endonuclease) and (E. coli) Exonuclease III (Exo III). Hybrids between the biotinylated oligonucleotide and ssDNA are formed in solution and then captured on streptavidin-coated paramagnetic beads. A magnet is used to retrieve the paramagnetic beads from the solution, leaving nonhybridized ssDNA behind. Subsequently, the captured ssDNA target is released from the biotinylated oligonucleotide that remains attached to the paramagnetic beads. After release, the desired cDNA clone is further enriched by using a nonbiotinylated target oligonucleotide to specifically prime conversion of the recovered ssDNA target to dsDNA. Following transformation and plating, typically, 20% to 100% of the colonies represent the cDNA clone of interest.

For ion31, the biotinylated oligonucleotide had the sequence as follows: TGCCAGTGAAAAGTGGGAATCGTGTCCCAT (SEQ ID NO:111). PCR primers having the sequence:

5′ CCCAGCCTCTATGTCATAAACC (SEQ ID NO:112) and:

3′ TCATGAGCAGGAAGACGTTG (SEQ ID NO:113) were used for library screening and colony screening, following the instructions provided by the manufacturer.

Two potentially full-length clones were isolated using this approach, ion31d6 and ion31c4. The two clones were sequenced, yielding the nucleotide sequences (SEQ ID NO: 103 and SEQ ID NO:104) and predicted amino acid sequences (SEQ ID NO:105 and SEQ ID NO:106) set forth in Table 5 and the attached Sequence Listing.

Analysis of the clones reveals high homology to ion1, filed in pending application Ser. No. 60/255,692 filed Dec. 15, 2000, which is incorporated by reference in its entirety, as well as to ion31, ion52, and ion111, described herein.

### Example 4

Northern Blot Analysis

Ion channel expression patterns can be determined through northern blot analysis of mRNA from different cell and tissue types. Typically, “blots” of isolated mRNA from such cells or tissues are prepared by standard methods or purchased, from commercial suppliers, and are subsequently probed with nucleotide probes representing a fragment of the polynucleotide encoding the ion channel polypeptide.

Those skilled in the art are familiar with standard PCR protocols for the generation of suitable probes using pairs of sense and antisense orientation oligonucleotide primers derived from SEQ ID NO:1 to SEQ ID NO:51 and SEQ ID NOS:103, 104, 107, and 108. During the PCR process, the probe is labeled radioactively with the use of α³²P-dCTP by Rediprime™ DNA labeling system (Amersham Pharmacia) so as to permit detection during analysis. The probe is further purified on a Nick Column (Amersham Pharmacia).

A multiple human tissue northern blot from Clontech (Human II#7767-1) is used in hybridization reactions with the probe to determine which tissues express ion channels. Pre-hybridization is carried out at 42° C. for 4 hours in 5×SSC, 1×Denhardt's reagent, 0.1% SDS, 50% formamide, 250 μg/ml salmon sperm DNA. Hybridization is performed overnight at 42° C. in the same mixture with the addition of about 1.5×10⁶ cpm/ml of labeled probe. The filters are washed several times at 42° C. in 0.2×SSC, 0.1% SDS. Filters were exposed to Kodak XAR film (Eastman Kodak Company, Rochester, N.Y., USA) with an intensifying screen at −80° C., allowing analysis of mRNA expression.

### Example 5

Expression of Ion Channel Polypeptides in Mammalian Cells

1. Expression of Ion Channel Polypeptides in HEK-293 cells

For expression of ion channel polypeptides in mammalian cells HEK-293 (transformed human, primary embryonic kidney cells), a plasmid bearing the relevant ion channel coding sequence is prepared, using vector pCDNA6 (Invitrogen). Vector pCDNA6 contains the CMV promoter and a blasticidin resistant gene for selection of stable transfectants. Many other vectors can be used containing, for example, different promoters, epitope tags for detection and/or purification of the protein, and resistance genes. The forward primer for amplification of this ion channel polypeptide encoding cDNA is determined by procedures as well known in the art and preferably contains a 5′ extension of nucleotides to introduce a restriction cloning site not present in the ion channel cDNA sequence, for example, a HindIII restriction site and nucleotides matching the ion channel nucleotide sequence. The reverse primer is also determined by procedures known in the art and preferably contains a 5′ extension of nucleotides to introduce a restriction cloning site not present in the ion channel cDNA sequence, for example, an XhoI restriction site, and nucleotides corresponding to the reverse complement of the ion channel nucleotide sequence. The PCR conditions are determined by the physical properties of the oligonucleotide primer and the length of the ion channel gene. The PCR product is gel purified and cloned into the HindIII-XhoI sites of the vector.

The plasmid DNA is purified using a Qiagen plasmid mini-prep kit and transfected into, for example, HEK-293 cells using DOTAP transfection media (Boehringer Mannhein, Indianapolis, Ind.). Transiently transfected cells are tested for ion channel activity and expression after 24-48 hours by established techniques of electrophysiology Electrophysiology, A Practical Approach, D I Wallis editor, IRL Press at Oxford University Press, (1993), and Voltage and patch Clamping with Microelectrodes, T G Smith, H Lecar, S J Redman and P W Gage, eds., Waverly Press, Inc for the American Physiology Society (1985). This provides one means by which ion channel activity can be characterized.

DNA is purified using Qiagen chromatography columns and transfected into HEK-293 cells using DOTAP transfection media (Boehringer Mannheim, Indianapolis, Ind.). Transiently transfected cells are tested for expression after 24 hours of transfection, using Western blots probed with anti-His and anti-ion channel peptide antibodies. Permanently transfected cells are selected with Zeocin and propagated. Production of the recombinant protein is detected from both cells and media by western blots probed with anti-His, anti-Myc or anti-ion channel peptide antibodies.

2. Expression of Ion Channel Polypeptides in COS Cells

For expression of ion channel polypeptides in COS7 cells, a polynucleotide molecule having a nucleotide of SEQ ID NO:1 to SEQ ID NO:51, and SEQ ID NOS:103, 104, 107, and 108, or complementary nucleotide sequences thereof, can be cloned into vector p3-CI. This vector is a pUC18-derived plasmid that contains the HCMV (human cytomegalovirus) intron located upstream from the bGH (bovine growth hormone) polyadenylation sequence and a multiple cloning site. In addition, the plasmid contains the dhrf (dihydrofolate reductase) gene which provides selection in the presence of the drug methotrexane (MTX) for selection of stable transformants. Many other vectors can be used containing, for example, different promoters, epitope tags for detection and/or purification of the protein, and resistance genes.

The forward primer is determined by procedures known in the art and preferably contains a 5′ extension which introduces an Xbal restriction site for cloning, followed by—nucleotides which correspond to a nucleotide sequence given in SEQ ID NO:1 to SEQ ID NO:51, and SEQ ID NOS:103, 104, 107, and 108, or portion thereof. The reverse primer is also determined by methods well known in the art and preferably contains a 5′—extension of nucleotides which introduces a SalI cloning site followed by nucleotides which correspond to the reverse complement of a nucleotide sequence given in SEQ ID NO:1 to SEQ ID NO:51, and SEQ ID NOS:103, 104, 107, and 108, or portion thereof.

The PCR consists of an initial denaturation step of 5 min at 95° C., 30 cycles of 30 sec denaturation at 95° C., 30 sec annealing at 58° C. and 30 sec extension at 72° C., followed by 5 min extension at 72° C. The PCR product is gel purified and ligated into the Xbal and SalI sites of vector p3-CI. This construct is transformed into E. coli cells for amplification and DNA purification. The DNA is purified with Qiagen chromatography columns and transfected into COS 7 cells using Lipofectamine™ reagent (Gibco/BRL), following the manufacturer's protocols. Forty-eight and 72 hours after transfection, the media and the cells are tested for recombinant protein expression.

Ion channel polypeptides expressed in cultured COS cells can be purified by disrupting cells via homogenization and purifying membranes by centrifugation, solubilizing the protein using a suitable detergent, and purifying the protein by, for example, chromatography. Purified ion channel is concentrated to 0.5 mg/ml in an Amicon concentrator fitted with a YM-10 membrane and stored at −80° C.

### Example 6

Expression of Ion Channel Polypeptides in Insect Cells

For expression of ion channel polypeptides in a baculovirus system, a polynucleotide molecule having a sequence selected from the group consisting of SEQ ID NO:1 to SEQ ID NO:51, and SEQ ID NOS:103, 104, 107, and 108, or a portion thereof, or complement thereof, is amplified by PCR. The forward primer is determined by methods known in the art and preferably constitutes a 5′ extension adding a NdeI cloning site, followed by nucleotides which corresponding to a nucleotide sequence provided in SEQ ID NO:1 to SEQ ID NO:51, and SEQ ID NOS:103, 104, 107, and 108, or a portion thereof. The reverse primer is also determined by methods known in the art and preferably constitutes a 5′ extension which introduces a KpnI cloning site, followed by nucleotides which correspond to the reverse complement of a nucleotide sequence provided in SEQ ID NO:1 to SEQ ID NO:51, and SEQ ID NOS:103, 104, 107, and 108, or a portion thereof.

The PCR product is gel purified, digested with NdeI and KpnI, and cloned into the corresponding sites of vector pACHTL-A (Pharmingen, San Diego, Calif.). The pAcHTL expression vector contains the strong polyhedrin promoter of the Autographa californica nuclear polyhedrosis virus (AcMNPV), and a 10OXHis tag upstream from the multiple cloning site. A protein kinase site for phosphorylation and a thrombin site for excision of the recombinant protein preceding the multiple cloning site is also present. Of course, many other baculovirus vectors can be used in place of pAcHTL-A, such as pAc373, pVL941 and pAcIM1. Other suitable vectors for the expression of ion channel polypeptides can be used, provided that such vector constructs include appropriately located signals for transcription, translation, and trafficking, such as an in-frame AUG and a signal peptide, as required. Such vectors are described in Luckow et al., Virology, 1989, 170, 31-39, among others.

The virus is grown and isolated using standard baculovirus expression methods, such as those described in Summers et al., A Manual of Methods for Baculovirus Vectors and Insect Cell Culture Procedures, Texas Agricultural Experimental Station Bulletin No. 1555 (1987).

In a preferred embodiment, pAcHLT-A containing the gene encoding the ion channel polypeptides is introduced into baculovirus using the “BaculoGold” transfection kit (Pharmingen, San Diego, Calif.) using methods provided by the manufacturer. Individual virus isolates are analyzed for protein production by radiolabeling infected cells with ³⁵S-methionine at 24 hours post infection. Infected cells are harvested at 48 hours post infection, and the labeled proteins are visualized by SDS-PAGE autoradiography. Viruses exhibiting high expression levels can be isolated and used for scaled up expression.

For expression of the ion channel polypeptides in Sf9 insect cells, a polynucleotide molecule having a sequence of SEQ ID NO:1 to SEQ ID NO:51, and SEQ ID NOS:103, 104, 107, and 108, or a portion thereof, is amplified by PCR using the primers and methods described above for baculovirus expression. The ion channel polypeptide encoding cDNA insert is cloned into vector pAcHLT-A (Pharmingen), between the NdeI and KpnI sites (after elimination of an internal NdeI site). DNA is purified using Qiagen chromatography columns. Preliminary Western blot experiments from non-purified plaques are tested for the presence of the recombinant protein of the expected size which reacts with the poly-His tag antibody. Because ion channel polypeptides are integral membrane proteins, preparation of the protein sample is facilitated using detergent extraction. Results are confirmed after further purification and expression optimization in HiG5 insect cells.

### Example 7

Interaction Trap/Two-Hybrid System

In order to assay for ion channel polypeptide-interacting proteins, the interaction trap/two-hybrid library screening method can be used. This assay was first described in Fields, et al., Nature, 1989, 340, 245, which is incorporated herein by reference in its entirety. A protocol is published in Current Protocols in Molecular Biology 1999, John Wiley & Sons, NY, and Ausubel, F. M. et al. 1992, Short Protocols in Molecular Biology, 4^{th} ed., Greene and Wiley-Interscience, New York, both of which are incorporated herein by reference in their entirety. Kits are available from Clontech, Palo Alto, Calif. (Matchmaker Two Hybrid System 3).

A fusion of the nucleotide sequences encoding all or a partial ion channel polypeptide and the yeast transcription factor GAL4 DNA-binding domain (DNA-BD) is constructed in an appropriate plasmid (i.e., pGBKT7), using standard subcloning techniques. Similarly, a GAL4 active domain (AD) fusion library is constructed in a second plasmid (i.e., pGADT7) from cDNA of potential ion channel polypeptide-binding proteins (for protocols on forming cDNA libraries, see Sambrook et al., supra. The DNA-BD/ion channel fusion construct is verified by sequencing, and tested for autonomous reporter gene activation and cell toxicity, both of which would prevent a successful two-hybrid analysis. Similar controls are performed with the AD/library fusion construct to ensure expression in host cells and lack of transcriptional activity. Yeast cells are transformed (ca. 10⁵ transformants/mg DNA) with both the ion channel and library fusion plasmids according to standard procedure (Ausubel, et al., supra). In vivo binding of DNA-BD/ion channel with AD/library proteins results in transcription of specific yeast plasmid reporter genes (i.e., lacZ, HIS3, ADE2, LEU2). Yeast cells are plated on nutrient-deficient media to screen for expression of reporter genes. Colonies are dually assayed for β-galactosidase activity upon growth in Xgal (5-bromo-4-chloro-3-indolyl-β-D-galactoside) supplemented media (filter assay for β-galactosidase activity is described in Breeden, et al., Cold Spring Harb. Symp. Quant. Biol., 1985, 50, 643, which is incorporated herein by reference in its entirety). Positive AD-library plasmids are rescued from transformants and reintroduced into the original yeast strain as well as other strains containing unrelated DNA-BD fusion proteins to confirm specific ion channel polypeptide/library protein interactions. Insert DNA is sequenced to verify the presence of an open reading frame fused to GAL4 AD and to determine the identity of the ion channel polypeptide-binding protein.

### Example 8

FRET Analysis of Protein-Protein Interactions Involving Ion Channel Polypeptides

In order to assay for ion channel polypeptide-interacting proteins, fluorescence resonance energy transfer (FRET) methods can be used. An example of this type of assay is described in Mahajan N P, et al., Nature Biotechnology, 1998, 16, 547, which is incorporated herein by reference in its entirety. This assay is based on the fact that when two fluorescent moieties having the appropriate excitation/emission properties are brought into close proximity, the donor fluorophore, when excited, can transfer its energy to the acceptor fluorophore whose emission is measured. The emission spectrum of the donor must overlap with the absorption spectrum of the acceptor while overlaps between the two absorption spectra and between the two emission spectra, respectively, should be minimized. An example of a useful donor/acceptor pair is Cyan Fluorescent Protein (CFP)/Yellow Fluorescent Protein (YFP) (Tsien, R Y (1998), Annual Rev Biochem 67, 509-544, which is incorporated by reference in its entirety).

A fusion of the nucleotide sequences encoding whole or partial ion channel polypeptides and CFP is constructed in an appropriate plasmid, using standard subcloning techniques. Similarly, a nucleotide encoding a YFP fusion of the possibly interacting target protein is constructed in a second plasmid. The CFP/ion channel polypeptide fusion construct is verified by sequencing. Similar controls are performed with the YFP/target protein construct. The expression of each protein can be monitored using fluorescence techniques (e.g., fluorescence microscopy or fluorescence spectroscopy). Host cells are transformed with both the CFP/ion channel polypeptide and YFP/target protein fusion plasmids according to standard procedure. In situ interactions between CFP/ion channel polypeptide and the YFP/target protein are detected by monitoring the YFP fluorescence after exciting the CFP fluorophore. The fluorescence is monitored using fluorescence microscopy or fluorescence spectroscopy. In addition, changes in the interaction due to e.g., external stimuli are measured using time-resolved fluorescence techniques.

Alternatively, a YFP fusion library may be constructed from cDNA of potential ion channel polypeptide-binding proteins (for protocols on forming cDNA libraries, see Sambrook et al., supra). Host cells are transformed with both the CFP/ion channel polypeptide and YFP fusion library plasmids. Clones exhibiting FRET are then isolated and the protein interacting with an ion channel polypeptide is identified by rescuing and sequencing the DNA encoding the YFP/target fusion protein.

### Example 9

Assays to Identify Modulators of Ion Channel Activity

Set forth below are several nonlimiting assays for identifying modulators (agonists and antagonists) of ion channel activity. Although the following assays typically measure calcium flux, it is contemplated that measurement of other ions may be made. Among the modulators that can be identified by these assays are natural ligand compounds of the ion channel; synthetic analogs and derivatives of natural ligands; antibodies, antibody fragments, and/or antibody-like compounds derived from natural antibodies or from antibody-like combinatorial libraries; and/or synthetic compounds identified by high-throughput screening of libraries; and the like. All modulators that bind ion channel are useful for identifying such ion channels in tissue samples (e.g., for diagnostic purposes, pathological purposes, and the like). Agonist and antagonist modulators are useful for up-regulating and down-regulating ion channel activity, respectively, to treat disease states characterized by abnormal levels of ion channels. The assays may be performed using single putative modulators, and/or may be performed using a known agonist in combination with candidate antagonists (or visa versa).

A. Aequorin Assays

In one assay, cells (e.g., CHO cells) are transiently co-transfected with both an ion channel expression construct and a construct that encodes the photoprotein apoaequorin. In the presence of the cofactor coelenterazine, apoaequorin will emit a measurable luminescence that is proportional to the amount of intracellular (cytoplasmic) free calcium. (See generally, Cobbold, et al. “Aequorin measurements of cytoplasmic free calcium,” In: McCormack J. G. and Cobbold P. H., eds., Cellular Calcium: A Practical Approach. Oxford:IRL Press (1991); Stables et al., Analytical Biochemistry 252: 115-26 (1997); and Haugland, Handbook of Fluorescent Probes and Research Chemicals. Sixth edition. Eugene Oreg.: Molecular Probes (1996).), each of which is incorporated by reference in its entirety.

In one exemplary assay, ion channel nucleic acid is subcloned into the commercial expression vector pzeoSV2 (Invitrogen) and transiently co-transfected along with a construct that encodes the photoprotein apoaquorin (Molecular Probes, Eugene, Oreg.) into CHO cells using the transfection reagent FuGENE 6 (Boehringer-Mannheim) and the transfection protocol provided in the product insert.

The cells are cultured for 24 hours at 37° C. in MEM (Gibco/BRL, Gaithersburg, Md.) supplemented with 10% fetal bovine serum, 2 mM glutamine, 10 U/ml penicillin and 10 μg/ml streptomycin, at which time the medium is changed to serum-free MEM containing 5 μM coelenterazine (Molecular Probes, Eugene, Oreg.). Culturing is then continued for two additional hours at 37° C. Subsequently, cells are detached from the plate using VERSENE (Gibco/BRL), washed, and resuspended at 200,000 cells/ml in serum-free MEM.

Dilutions of candidate ion channel modulator compounds are prepared in serum-free MEM and dispensed into wells of an opaque 96-well assay plate at 50 μl/well. Plates are then loaded onto an MLX microtiter plate luminometer (Dynex Technologies, Inc., Chantilly, Va.). The instrument is programmed to dispense 50μl cell suspensions into each well, one well at a time, and immediately read luminescence for 15 seconds. Dose-response curves for the candidate modulators are constructed using the area under the curve for each light signal peak. Data are analyzed with SlideWrite, using the equation for a one-site ligand, and EC₅₀ values are obtained. Changes in luminescence caused by the compounds are considered indicative of modulatory activity.

B. Intracellular Calcium Measurement Using FLIPR

Changes in intracellular calcium levels are another recognized indicator of ion channel activity, and such assays can be employed to screen for modulators of ion channel activity. For example, CHO cells stably transfected with an ion channel expression vector are plated at a density of 4×10⁴ cells/well in Packard black-walled, 96-well plates specially designed to discriminate fluorescence signals emanating from the various wells on the plate. The cells are incubated for 60 minutes at 37° C. in modified Dulbecco's PBS (D-PBS) containing 36 mg/L pyruvate and 1 g/L glucose with the addition of 1% fetal bovine serum and one of four calcium indicator dyes (Fluo-3™ AM, Fluo-4™ AM, Calcium Green™-1 AM, or Oregon Green™ 488 BAPTA-1 AM), each at a concentration of 4μM. Plates are washed once with modified D-PBS without 1% fetal bovine serum and incubated for 10 minutes at 37° C. to remove residual dye from the cellular membrane. In addition, a series of washes with modified D-PBS without 1% fetal bovine serum is performed immediately prior to activation of the calcium response.

A calcium response is initiated by the addition of one or more candidate receptor agonist compounds, calcium ionophore A23187 (10 μM; positive control), or ATP (4 μM; positive control). Fluorescence is measured by Molecular Device's FLIPR with an argon laser (excitation at 488 nm). (See, e.g., Kuntzweiler et al., Drug Development Research, 44(1):14-20 (1998)). The F-stop for the detector camera was set at 2.5 and the length of exposure was 0.4 milliseconds. Basal fluorescence of cells was measured for 20 seconds prior to addition of candidate agonist, ATP, or A23187, and the basal fluorescence level was subtracted from the response signal. The calcium signal is measured for approximately 200 seconds, taking readings every two seconds. Calcium ionophore A23187 and ATP increase the calcium signal 200% above baseline levels.

C. Extracellular Acidification Rate

In yet another assay, the effects of candidate modulators of ion channel activity are assayed by monitoring extracellular changes in pH induced by the test compounds. (See, e.g., Dunlop et al., Journal of Pharmacological and Toxicological Methods 40(1):47-55 (1998).) In one embodiment, CHO cells transfected with an ion channel expression vector are seeded into 12 mm capsule cups (Molecular Devices Corp.) at 4×10⁵ cells/cup in MEM supplemented with 10% fetal bovine serum, 2mM L-glutamine, 10 U/ml penicillin, and 10 μg/ml streptomycin. The cells are incubated in this medium at 37° C. in 5% CO₂ for 24 hours.

Extracellular acidification rates are measured using a Cytosensor microphysiometer (Molecular Devices Corp.). The capsule cups are loaded into the sensor chambers of the microphysiometer and the chambers are perfused with running buffer (bicarbonate-free MEM supplemented with 4mM L-glutamine, 10 units/ml penicillin, 10 μg/ml streptomycin, 26 mM NaCl) at a flow rate of 100 μl/minute. Candidate agonists or other agents are diluted into the running buffer and perfused through a second fluid path. During each 60-second pump cycle, the pump is run for 38 seconds and is off for the remaining 22 seconds. The pH of the running buffer in the sensor chamber is recorded during the cycle from 43-58 seconds, and the pump is re-started at 60 seconds to start the next cycle. The rate of acidification of the running buffer during the recording time is calculated by the Cytosoft program. Changes in the rate of acidification are calculated by subtracting the baseline value (the average of 4 rate measurements immediately before addition of a modulator candidate) from the highest rate measurement obtained after addition of a modulator candidate. The selected instrument detects 61 mV/pH unit. Modulators that act as agonists of the ion channel result in an increase in the rate of extracellular acidification compared to the rate in the absence of agonist. This response is blocked by modulators which act as antagonists of the ion channel.

### Example 10

High Throughput Screening for Modulators of Ion Channels Using FLIPR

One method to identify compounds that modulate the activity of an ion channel polypeptide is through the use of the FLIPR Fluorometric Imaging Plate Reader system. This system was developed by Dr. Vince Groppi of the Pharmacia Corporation to perform cell-based, high-throughput screening (HTS) assays measuring, for example, membrane potential. Changes in plasma membrane potential correlate with the modulation of ion channels as ions move into or out of the cell. The FLIPR system measures such changes in membrane potential. This is accomplished by loading cells expressing an ion channel gene with a cell-membrane permeant fluorescent indicator dye suitable for measuring changes in membrane potential such as diBAC (bis-(1,3-dibutylbarbituric acid) pentamethine oxonol, Molecular Probes). Thus the modulation of ion channel activity is assessed with FLIPR and detected as changes in the emission spectrum of the diBAC dye.

As an example, COS cells that have been transfected with an ion channel gene of interest are bathed in diBAC. Due to the presence of both endogenous potassium channels in the cells as well as the transfected channel, the addition of 30 mM extracellular potassium causes membrane depolarization which results in an increase in diBAC uptake by the cell, and thus an overall increase in fluorescence. When cells are treated with a potassium channel opener, such as chromakalim, the membrane is hyperpolarized, causing a net outflow of diBAC, and thus a reduction in fluorescence. In this manner the effect of unknown test compounds on membrane potential can be assessed using this assay.

### Example 11

Chimeric Receptors

A chimeric receptor can be used to measure the activity of ligand binding when the ligand's native receptor activity is not amenable to easy measurement. Such chimera may consist of a ligand-binding domain of one receptor fused to the pore-forming domain of another receptor. A useful example of such a chimera can be found in WO 00/73431 A2.

The pore-forming transmembrane domain of ion-34 (SEQ ID NO:4) can be fused, for example, with the extracellular domain of the alpha7 nicotinic acetylcholine receptor to form a chimeric receptor that binds alpha7 receptor ligands but passes current like that of ion-66. To generate this chimera, PCR primers are designed to amplify the 5′ region of the alpha7 receptor (GenBank accession number U62436) with a region of overlap with ion-66 on the 3′-most primer.

PCR is performed using the appropriate cDNA clone as a template using Platinum Taq polymerase (Life Technologies, Gaithersburg, Md.) according to the manufacturer's instructions. The PCR products from these two reactions are then diluted 1:1000 and pooled in a second PCR mixture with appropriately designed primers to generate the final chimeric cDNA by splice-overlap PCR. These primers also add an EcoRI restriction site to the 5′ end and a NotI site to the 3′ end to facilitate subcloning into pcDNA3.1 (Invitrogen). The PCR product is ligated into pcDNA3.1 and transformed into competent E. coli (Life Technologies, Gaithersburg, Md.). Isolated E. coli colonies selected on ampicillin-containing medium are isolated and expanded. The DNA from the plasmid in E. coli is isolated and sequenced to verify that the expected sequences are obtained. The DNA is then transformed into mammalian cells such as SH-EP1 cells using cationic lipid transfection reagent. Cells that are stably transformed are selected in the presence of 800 micrograms/ml geneticin. These cells are then assayed as described supra for changes in intracellular calcium or changes in membrane potential in response to ligands, e.g. nicotine.

### Example 12

Tissue Expression Profiling

Tissue expression profiling of ion31 and ion52 was tested by PCR using genomic DNA as a template in a 100 μL reaction mixture containing: 0.5 mM each forward and reverse primer, 1×PCR buffer II (Perkin-Elmer), 1.5 mM MgCl₂ (Perkin-Elmer), 0.2 mM each dNTP (Gibco-BRL), 0.5 mg human genomic DNA (Clontech) and 5 units AmpliTaq Gold (Perkin-Elmer) with the following thermocycling conditions in a Perkin-Elmer 9600 thermocycler: one cycle of 95° C. for 10 minutes; 35 cycles of 94° C. for 30 seconds, 55° C. for 30 seconds, 72° C. for 1 minute; one cycle of 72° C. for 10 minutes followed by a 4° C. soak.

Products were analyzed on 2% agarose gels containing 0.5 mg/mL ethidium bromide run in Tris-Acetate EDTA running buffer (Gibco-BRL). cDNAs from human tissue cDNA were panels obtained from OriGene (Rockville, Md.).

Ion31

The forward primer used was to detect expression of ion-31 was:

5′ GCCATCAGGCGCAGGCCAA (SEQ ID NO:114), and the reverse primer was:

5′ CAAGTCATTCATCATGAGCAGGA 3′ (SEQ ID NO:115).

Ion31 mRNA was detected in tissues including testis, retina, lung, brain, fetal brain, muscle, kidney, and small intestine. This indicates that compounds modulating the activity of ion31 may be useful in the treatment of diseases including but not limited to Alzheimer's disease, Parkinson's disease, schizophrenia, depression, anxiety, migraine, epilepsy, obesity, bipolar and other mood disorders, inflammatory bowel disease, diarrhea or constipation, asthma, arthritis, leukemias and lymphomas, neurodegeneration, retinal degeneration, restenosis, cardiac arrythmia, diabetes, hair growth, high blood pressure, pain, and nausea due to chemotherapy or anti-emesis.

Ion52

The forward primer used was to detect expression of ion-52 was:

TGCCTGTCCCTGATGGTGGG (SEQ ID NO:116), and the reverse primer was:

GAGCAGCAGGGAGTGGAGC (SEQ ID NO:117).

Ion52 mRNA was detected in tissues including brain, fetal brain, heart, kidney, lung, small intestine, muscle, spleen, peripheral blood leukocytes, testis, and retina. This indicates that compounds modulating the activity of ion52 may be useful in the treatment of diseases including but not limited to Alzheimer's disease, Parkinson's disease, schizophrenia, depression, anxiety, migraine, epilepsy, obesity, bipolar and other mood disorders, inflammatory bowel disease, diarrhea or constipation, asthma, arthritis, leukemias and lymphomas, neurodegeneration, retinal degeneration, restenosis, cardiac arrythmia, diabetes, hair growth, high blood pressure, pain, and nausea due to chemotherapy or anti-emesis.

Tissue specific expression of cDNAs encoding other ion-x may be accomplished using similar methods.

As those skilled in the art will appreciate, numerous changes and modifications may be made to the preferred embodiments of the invention without departing from the spirit of the invention. It is intended that all such variations fall within the scope of the invention. The entire disclosure of each publication cited herein is hereby incorporated by reference.

What is claimed is:

1. An isolated nucleic acid molecule comprising a nucleotide sequence that encodes a polypeptide comprising an amino acid sequence homologous to a sequence selected from the group consisting of SEQ ID NO:52 to SEQ ID NO:102 and SEQ ID NOS:109 and 110, said nucleic acid molecule encoding at least a portion of ion-x.

2. The isolated nucleic acid molecule of claim 1 comprising a sequence that encodes a polypeptide comprising a sequence selected from the group consisting of SEQ ID NO:52 to SEQ ID NO:102 and SEQ ID NOS:109 and 110.

3. The isolated nucleic acid molecule of claim 1 comprising a sequence homologous to a sequence selected from the group consisting of SEQ ID NO:1 to SEQ ID NO:51 and SEQ ID NOS:107 and 108.

4. The isolated nucleic acid molecule of claim 1 comprising a sequence selected from the group consisting of SEQ ID NO:1 to SEQ ID NO:51 and SEQ ID NOS:107 and 108.

5. The isolated nucleic acid molecule of claim 1 wherein said nucleic acid molecule is DNA.

6. The isolated nucleic acid molecule of claim 1 wherein said nucleic acid molecule is RNA.

7. An expression vector comprising a nucleic acid molecule of any one of claims 1 to 4.

8. The expression vector of claim 7 wherein said nucleic acid molecule comprises a sequence selected from the group consisting of SEQ ID NO:1 to SEQ ID NO:51 and SEQ ID NOS:107 and 108.

9. The expression vector of claim 7 wherein said vector is a plasmid.

10. The expression vector of claim 7 wherein said vector is a viral particle.

11. The expression vector of claim 10 wherein said vector is selected from the group consisting of adenoviruses, baculoviruses, parvoviruses, herpesviruses, poxviruses, adeno-associated viruses, Semliki Forest viruses, vaccinia viruses, and retroviruses.

12. The expression vector of claim 7 wherein said nucleic acid molecule is operably connected to a promoter selected from the group consisting of simian virus 40, mouse mammary tumor virus, long terminal repeat of human immunodeficiency virus, maloney virus, cytomegalovirus immediate early promoter, Epstein Barr virus, rous sarcoma virus, human actin, human myosin, human hemoglobin, human muscle creatine, and human metalothionein.

13. A host cell transformed with an expression vector of claim 8.

14. The transformed host cell of claim 13 wherein said cell is a bacterial cell.

15. The transformed host cell of claim 14 wherein said bacterial cell is E. coli.

16. The transformed host cell of claim 13 wherein said cell is yeast.

17. The transformed host cell of claim 16 wherein said yeast is S. cerevisiae.

18. The transformed host cell of claim 13 wherein said cell is an insect cell.

19. The transformed host cell of claim 18 wherein said insect cell is S. frugiperda.

20. The transformed host cell of claim 13 wherein said cell is a mammalian cell.

21. The transformed host cell of claim 20 wherein mammalian cell is selected from the group consisting of chinese hamster ovary cells, HeLa cells, African green monkey kidney cells, human HEK-293 cells, and murine 3T3 fibroblasts.

22. An isolated nucleic acid molecule comprising at least 10 nucleotides, said nucleic acid molecule comprising a nucleotide sequence complementary to a sequence selected from the group consisting of SEQ ID NO:1 to SEQ ID NO:51, and SEQ ID NOS:107 and 108.

23. The nucleic acid molecule of claim 22 wherein said molecule is an antisense oligonucleotide directed to a region of a sequence selected from the group consisting of SEQ ID NO:1 to SEQ ID NO:51, and SEQ ID NOS:107 and 108.

24. The nucleic acid molecule of claim 23 wherein said oligonucleotide is directed to a regulatory region of a sequence selected from the group consisting of SEQ ID NO:1 to SEQ ID NO:51, and SEQ ID NOS:107 and 108.

25. A composition comprising a nucleic acid molecule of any one of claims 1 to 4 or 22 and an acceptable carrier or diluent.

26. A composition comprising a recombinant expression vector of claim 7 and an acceptable carrier or diluent.

27. A method of producing a polypeptide that comprises a sequence selected from the group consisting of SEQ ID NO:52 to SEQ ID NO:102 and SEQ ID NOS:109 and 110, said method comprising the steps of:
a) introducing a recombinant expression vector of claim 7 into a compatible host cell;
b) growing said host cell under conditions for expression of said polypeptide; and
c) recovering said polypeptide.

28. The method of claim 27 wherein said host cell is lysed and said polypeptide is recovered from the lysate of said host cell.

29. The method of claim 27 wherein said polypeptide is recovered by purifying the culture medium without lysing said host cell.

30. An isolated polypeptide encoded by a nucleic acid molecule of claim 1.

31. The polypeptide of claim 30 wherein said polypeptide comprises a sequence selected from the group consisting of SEQ ID NO:52 to SEQ ID NO:102 and SEQ ID NOS:109 and 110.

32. The polypeptide of claim 30 wherein said polypeptide comprises an amino acid sequence homologous to a sequence selected from the group consisting of SEQ ID NO:52 to SEQ ID NO:102 and SEQ ID NOS:109 and 110.

33. The polypeptide of claim 30 wherein said sequence homologous to a sequence selected from the group consisting of SEQ ID NO:52 to SEQ ID NO:102 and SEQ ID NOS:109 and 110, comprises at least one conservative amino acid substitution compared to the sequence selected from the group consisting of SEQ ID NO:52 to SEQ ID NO:102 and SEQ ID NOS:109 and 110.

34. The polypeptide of claim 30 wherein said polypeptide comprises an allelic variant of a polypeptide with a sequence selected from the group consisting of SEQ ID NO:52 to SEQ ID NO:102 and SEQ ID NOS:109 and 110.

35. A composition comprising a polypeptide of claim 30 and an acceptable carrier or diluent.

36. An isolated antibody which binds to an epitope on a polypeptide of claim 30.

37. The antibody of claim 36 wherein said antibody is a monoclonal antibody.

38. A composition comprising an antibody of claim 36 and an acceptable carrier or diluent.

39. A method of inducing an immune response in a mammal against a polypeptide of claim 30 comprising administering to said mammal an amount of said polypeptide sufficient to induce said immune response.

40. A method for identifying a compound which binds ion-x comprising the steps of:
a) contacting ion-x with a compound; and
b) determining whether said compound binds ion-x.

41. The method of claim 40 wherein the ion-x comprises an amino acid sequence selected from the group consisting of SEQ ID NO:52 to SEQ ID NO:102 and SEQ ID NOS:109 and 110.

42. The method of claim 40 wherein binding of said compound to ion-x is determined by a protein binding assay.

43. The method of claim 40 wherein said protein binding assay is selected from the group consisting of a gel-shift assay, Western blot, radiolabeled competition assay, phage-based expression cloning, co-fractionation by chromatography, co-precipitation, cross linking, interaction trap/two-hybrid analysis, southwestern analysis, and ELISA.

44. A compound identified by the method of claim 40.

45. A method for identifying a compound which binds a nucleic acid molecule encoding ion-x comprising the steps of:
a) contacting said nucleic acid molecule encoding ion-x with a compound; and
b) determining whether said compound binds said nucleic acid molecule.

46. The method of claim 45 wherein binding is determined by a gel-shift assay.

47. A compound identified by the method of claim 45.

48. A method for identifying a compound which modulates the activity of ion-x comprising the steps of:
a) contacting ion-x with a compound; and
b) determining whether ion-x activity has been modulated.

49. The method of claim 48 wherein the ion-x comprises an amino acid sequence selected from the group consisting of: SEQ ID NO:52 to SEQ ID NO:102 and SEQ ID NOS:109 and 110.

50. The method of claim 48 wherein said activity is neuropeptide binding.

51. The method of claim 48 wherein said activity is neuropeptide signaling.

52. A compound identified by the method of claim 48.

53. A method of identifying an animal homolog of ion-x comprising the steps:
a) comparing the nucleic acid sequences of the animal with a sequence selected from the group consisting of SEQ ID NO:1 to SEQ ID NO:51, and SEQ ID NOS:107 and 108; and
b) identifying nucleic acid sequences of the animal that are homologous to said sequence selected from the group consisting of SEQ ID NO:1 to SEQ ID NO:51, and SEQ ID NOS:107 and 108.

54. The method of claim 53 wherein comparing the nucleic acid sequences of the animal with a sequence selected from the group consisting of SEQ ID NO:1 to SEQ ID NO:51, and SEQ ID NOS:107 and 108, is performed by DNA hybridization.

55. The method of claim 53 wherein comparing the nucleic acid sequences of the animal with a sequence selected from the group consisting of SEQ ID NO:1 to SEQ ID NO:51, and SEQ ID NOS:107 and 108, is performed by computer homology search.

56. A method of screening a human subject to diagnose a disorder affecting the brain or genetic predisposition therefor, comprising the steps of:
(a) assaying nucleic acid of a human subject to determine a presence or an absence of a mutation altering an amino acid sequence, expression, or biological activity of at least one ion channel that is expressed in the brain, wherein the ion channel comprises an amino acid sequence selected from the group consisting of SEQ ID NO:52 to SEQ ID NO:102 and SEQ ID NOS:109 and 110, and allelic variants thereof, and wherein the nucleic acid corresponds to a gene encoding the ion channel; and
(b) diagnosing the disorder or predisposition from the presence or absence of said mutation, wherein the presence of a mutation altering the amino acid sequence, expression, or biological activity of the ion channel correlates with an increased risk of developing the disorder.

57. A method according to claim 56, wherein the assaying step comprises at least one procedure selected from the group consisting of:
a) comparing nucleotide sequences from the human subject and reference sequences and determining a difference of at least a nucleotide of at least one codon between the nucleotide sequences from the human subject that encodes an ion-x allele and an ion-x reference sequence;
(b) performing a hybridization assay to determine whether nucleic acid from the human subject has a nucleotide sequence identical to or different from one or more reference sequences;
(c) performing a polynucleotide migration assay to determine whether nucleic acid from the human subject has a nucleotide sequence identical to or different from one or more reference sequences; and
(d) performing a restriction endonuclease digestion to determine whether nucleic acid from the human subject has a nucleotide sequence identical to or different from one or more reference sequences.

58. A method of screening for an ion-x mental disorder genotype in a human patient, comprising the steps of:
(a) providing a biological sample comprising nucleic acid from said patient, said nucleic acid including sequences corresponding to alleles of ion-x; and
(b) detecting the presence of one or more mutations in the ion-x alleles;
wherein the presence of a mutation in an ion-x allele is indicative of a mental disorder genotype.

59. The method according to claim 58 wherein said biological sample is a cell sample.

60. The method according to claim 58 wherein said nucleic acid is DNA.

61. The method according to claim 58 wherein said nucleic acid is RNA.

62. A kit for screening a human subject to diagnose a mental disorder or a genetic predisposition therefor, comprising, in association:
(a) an oligonucleotide useful as a probe for identifying polymorphisms in a human ion-x gene, the oligonucleotide comprising 6-50 nucleotides in a sequence that is identical or complementary to a sequence of a wild type human ion-x coding sequence, except for one sequence difference selected from the group consisting of a nucleotide addition, a nucleotide deletion, or nucleotide substitution; and
(b) a media packaged with the oligonucleotide, said media containing information for identifying polymorphisms that correlate with a mental disorder or a genetic predisposition therefor, the polymorphisms being identifiable using the oligonucleotide as a probe.

63. A method of identifying an ion channel allelic variant that correlates with a mental disorder, comprising steps of:
(a) providing a biological sample comprising nucleic acid from a human patient diagnosed with a mental disorder, or from the patient's genetic progenitors or progeny;
(b) detecting in the nucleic acid the presence of one or more mutations in an ion channel that is expressed in the brain, wherein the ion channel comprises an amino acid sequence selected from the group consisting of SEQ ID NO:52 to SEQ ID NO:102 and SEQ ID NOS:109 and 110, and allelic variants thereof, and wherein the nucleic acid includes sequence corresponding to the gene or genes encoding the ion channel;
wherein the one or more mutations detected indicates an allelic variant that correlates with a mental disorder.

64. A purified and isolated polynucleotide comprising a nucleotide sequence encoding ion-x allelic variant identified according to claim 63.

65. A host cell transformed or transfected with a polynucleotide according to claim 64 or with a vector comprising the polynucleotide.

66. A purified polynucleotide comprising a nucleotide sequence encoding ion-x of a human with a mental disorder;
wherein said polynucleotide hybridizes to the complement of a sequence selected from the group consisting of SEQ ID NO:52 to SEQ ID NO:102 and SEQ ID NOS:109 and 110 under the following hybridization conditions:
(a) hybridization for 16 hours at 42° C. in a hybridization solution comprising 50% formamide, 1% SDS, 1 M NaCl, 10% dextran sulfate and
(b) washing 2 times for 30 minutes at 60° C. in a wash solution comprising 0.1×SSC and 1% SDS; and
wherein the polynucleotide that encodes ion-x amino acid sequence of the human differs from the sequence selected from the group consisting of SEQ ID NO:52 to SEQ ID NO:102 and SEQ ID NOS:109 and 110 by at least one residue.

67. A vector comprising a polynucleotide according to claim 66.

68. A host cell that has been transformed or transfected with a polynucleotide according to claim 66 and that expresses the ion-x protein encoded by the polynucleotide.

69. A method for identifying a modulator of biological activity of ion-x comprising the steps of:
a) contacting a cell according to claim 68 in the presence and in the absence of a putative modulator compound;
b) measuring ion-x biological activity in the cell;
wherein decreased or increased ion-x biological activity in the presence versus absence of the putative modulator is indicative of a modulator of biological activity.

70. A method to identify compounds useful for the treatment of a disorder, said method comprising the steps of:
(a) contacting a composition comprising ion-x with a compound suspected of binding ion-x;
(b) detecting binding between ion-x and the compound suspected of binding ion-x;
wherein compounds identified as binding ion-x are candidate compounds useful for the treatment of a disorder.

71. A method for identifying a compound useful as a modulator of binding between ion-x and a binding partner of ion-x comprising the steps of:
(a) contacting the binding partner and a composition comprising ion-x in the presence and in the absence of a putative modulator compound;
(b) detecting binding between the binding partner and ion-x;
wherein decreased or increased binding between the binding partner and ion-x in the presence of the putative modulator, as compared to binding in the absence of the putative modulator is indicative a modulator compound useful for the treatment of a disorder.

72. A method according to claim 70 or 71 wherein the composition comprises a cell expressing ion-x on its surface.

73. A method according to claim 72 wherein the composition comprises a cell transformed or transfected with a polynucleotide that encodes ion-x.

74. A chimeric receptor comprising at least 5 amino acid residues, said receptor comprising at least a portion of a sequence selected from the group consisting of SEQ ID NO:52 to SEQ ID NO:102, and SEQ ID NOS:109 and 110.

75. An isolated nucleic acid molecule comprising a nucleotide sequence that encodes a polypeptide comprising an amino acid sequence homologous to a sequence selected from the group consisting of SEQ ID NO:52 to SEQ ID NO:102 and SEQ ID NOS:109 and 110, said nucleic acid molecule encoding at least a portion of ion-x.

76. An isolated nucleic acid molecule comprising a nucleotide sequence that encodes a polypeptide comprising an amino acid sequence homologous to a sequence selected from the group consisting of SEQ ID NO:105 and SEQ ID NO:106.

77. An isolated nucleic acid molecule comprising a nucleotide sequence homologous to a sequence selected from the group consisting of SEQ ID NO:103 and SEQ ID NO:104.

78 An isolated nucleic acid molecule comprising a nucleotide sequence selected from the group consisting of SEQ ID NO:103 and SEQ ID NO:104.

79. The isolated nucleic acid molecule of claim 76 wherein said nucleic acid molecule is DNA.

80. The isolated nucleic acid molecule of claim 76 wherein said nucleic acid molecule is RNA.

81. An expression vector comprising a nucleic acid molecule of any one of claims 76 to 78.

82. The expression vector of claim 81 wherein said nucleic acid molecule comprises a sequence selected from the group consisting of SEQ ID NO:103 and SEQ ID NO:104.

83 A host cell transformed with an expression vector of claim 81.

84 An isolated nucleic acid molecule comprising at least 10 nucleotides, said nucleic acid molecule comprising a nucleotide sequence complementary to a sequence selected from the group consisting of SEQ ID NO:103 and SEQ ID NO:104.

85. The nucleic acid molecule of claim 84 wherein said molecule is an antisense oligonucleotide directed to a region of a sequence selected from the group consisting of SEQ ID NO:103 and SEQ ID NO:104.

86. The nucleic acid molecule of claim 85 wherein said oligonucleotide is directed to a regulatory region of a sequence selected from the group consisting of SEQ ID NO:103 and SEQ ID NO:104.

87. A composition comprising a recombinant expression vector of claim 81 and an acceptable carrier or diluent.

88. A method of producing a polypeptide that comprises a sequence selected from the group consisting of SEQ ID NO:105 and SEQ ID NOS:106, said method comprising the steps of:
a) introducing a recombinant expression vector of claim 81 into a compatible host cell;
b) growing said host cell under conditions for expression of said polypeptide; and
c) recovering said polypeptide.

89. An isolated polypeptide encoded by a nucleic acid molecule of claim 76.

90. The polypeptide of claim 89 wherein said polypeptide comprises a sequence selected from the group consisting of SEQ ID NO:105 and SEQ ID NOS:106.

91. The polypeptide of claim 89 wherein said polypeptide comprises an amino acid sequence homologous to a sequence selected from the group consisting of SEQ ID NO:105 and SEQ ID NOS:106.

92. The polypeptide of claim 89 wherein said sequence homologous to a sequence selected from the group consisting of SEQ ID NO:105 and SEQ ID NOS:106, comprises at least one conservative amino acid substitution compared to the sequence selected from the group consisting of SEQ ID NO:105 and SEQ ID NOS:106.

93. The polypeptide of claim 89 wherein said polypeptide comprises an allelic variant of a polypeptide with a sequence selected from the group consisting of SEQ ID NO:105 and SEQ ID NOS:106.

94. A composition comprising a polypeptide of claim 89 and an acceptable carrier or diluent.

95. An isolated antibody which binds to an epitope on a polypeptide of claim 83.

96. The antibody of claim 95 wherein said antibody is a monoclonal antibody.

97. A method of inducing an immune response in a mammal against a polypeptide of claim 89 comprising administering to said mammal an amount of said polypeptide sufficient to induce said immune response.

98. A method for identifying a compound which binds an ion channel encoded by a sequence selected from the group consisting of SEQ ID NO:103 and SEQ ID NO:104 comprising the steps of:
a) contacting said ion channel with a compound; and
c) determining whether said compound binds said ion channel.

99. The method of claim 98 wherein the ion channel comprises an amino acid sequence selected from the group consisting of SEQ ID NO:105 and SEQ ID NO:106.

100. A compound identified by the method of claim 98.

101. A method for identifying a compound which binds a nucleic acid molecule having a sequence selected from the group consisting of SEQ ID NO:103 and SEQ ID NO:104 comprising the steps of:
a) contacting said nucleic acid molecule with a compound; and
b) determining whether said compound binds said nucleic acid molecule.

102. A compound identified by the method of claim 101.

103. A method for identifying a compound which modulates the activity of an ion channel encoded by a sequence selected from the group consisting of SEQ ID NO:103 and SEQ ID NO:104 comprising the steps of:
a) contacting said ion channel with a compound; and
b) determining whether ion channel activity has been modulated.

104. The method of claim 103 wherein the ion channel comprises an amino acid sequence selected from the group consisting of SEQ ID NO:105 and SEQ ID NO:106.

105. A compound identified by the method of claim 103.

106. A method of screening a human subject to diagnose a disorder affecting the brain or genetic predisposition therefor, comprising the steps of:
(a) assaying nucleic acid of a human subject to determine a presence or an absence of a mutation altering an amino acid sequence, expression, or biological activity of at least one ion channel that is expressed in the brain, wherein the ion channel comprises an amino acid sequence selected from the group consisting of SEQ ID NO:105 and SEQ ID NO:106, and allelic variants thereof, and wherein the nucleic acid corresponds to a gene encoding the ion channel; and
(b) diagnosing the disorder or predisposition from the presence or absence of said mutation, wherein the presence of a mutation altering the amino acid sequence, expression, or biological activity of the ion channel correlates with an increased risk of developing the disorder.

107. A kit for screening a human subject to diagnose a mental disorder or a genetic predisposition therefor, comprising, in association:
(a) an oligonucleotide useful as a probe for identifying polymorphisms in a human ion channel gene, the oligonucleotide comprising 6-50 nucleotides in a sequence that is identical or complementary to a sequence selected from the group consisting of SEQ ID NO:103 and SEQ ID NO:104, except for one sequence difference selected from the group consisting of a nucleotide addition, a nucleotide deletion, or nucleotide substitution; and
(b) a media packaged with the oligonucleotide, said media containing information for identifying polymorphisms that correlate with a mental disorder or a genetic predisposition therefor, the polymorphisms being identifiable using the oligonucleotide as a probe.

108. A method of identifying an ion channel allelic variant that correlates with a mental disorder, comprising steps of:
(a) providing a biological sample comprising nucleic acid from a human patient diagnosed with a mental disorder, or from the patient's genetic progenitors or progeny;
(b) detecting in the nucleic acid the presence of one or more mutations in an ion channel that is expressed in the brain, wherein the ion channel comprises an amino acid sequence selected from the group consisting of SEQ ID NO:105 and SEQ ID NO:106, and allelic variants thereof, and wherein the nucleic acid includes sequence corresponding to the gene or genes encoding the ion channel;
wherein the one or more mutations detected indicates an allelic variant that correlates with a mental disorder.

109. A purified and isolated polynucleotide comprising a nucleotide sequence encoding ion-x allelic variant identified according to claim 108.

110. A host cell transformed or transfected with a polynucleotide according to claim 109 or with a vector comprising the polynucleotide.

111. A method for identifying a modulator of biological activity of an ion channel encoded by a sequence selected from the group consisting of SEQ ID NO:103 and SEQ ID NO:104 comprising the steps of:
a) contacting a cell according to claim 110 in the presence and in the absence of a putative modulator compound;
b) measuring ion channel biological activity in the cell;
wherein decreased or increased ion channel biological activity in the presence versus absence of the putative modulator is indicative of a modulator of biological activity.

112. A method to identify compounds useful for the treatment of a disorder, said method comprising the steps of:
(a) contacting a composition comprising an ion channel encoded by a sequence selected from the group consisting of SEQ ID NO:103 and SEQ ID NO:104 with a compound suspected of binding said ion channel;
(b) detecting binding between said ion channel and the compound suspected of binding said ion channel;
wherein compounds identified as binding said ion channel are candidate compounds useful for the treatment of a disorder.

113. A method for identifying a compound useful as a modulator of binding between an ion channel encoded by a sequence selected from the group consisting of SEQ ID NO:103 and SEQ ID NO:104 and a binding partner of said ion channel comprising the steps of:
(a) contacting the binding partner and a composition comprising said ion channel in the presence and in the absence of a putative modulator compound;
(b) detecting binding between the binding partner and said ion channel;
wherein decreased or increased binding between the binding partner and said ion channel in the presence of the putative modulator, as compared to binding in the absence of the putative modulator is indicative a modulator compound useful for the treatment of a disorder.

114. A method according to claim 112 or 113 wherein the composition comprises a cell expressing said ion channel on its surface.

115. A method according to claim 114 wherein the composition comprises a cell transformed or transfected with a polynucleotide that encodes said ion channel.

116. A chimeric receptor comprising at least 5 amino acid residues, said receptor comprising at least a portion of a sequence selected from the group consisting of SEQ ID NO:105 and SEQ ID NOS:106.


SEQUENCE LISTING (117 sequences)

SEQ ID NO: 1 - DNA, 574 bp, Homo sapiens
ccctcctccctggccccgggtgccctttctcctcctgaagtgggaggagccatactgatgaggggggtgccactggcaggggagcaagtcattcatcatgagcaggaagacgttgtagcccagcagaagtgttatcttgaatggggcacgattctcgctctctgctggcaggtagaagctgagggcatcaatggcaaccagaaagctactgggcaccagcaggtttatgatgtagaggcttggcctgcgcctgatggccacctggagagagaccggagaagatggcaaataaatagatgtcagagggctcaatttgtatatctgacccctaatctttgccaatgtgctgtgaggctgctggggacgatctttttaagtaacacttttgcatataattgtgctcgcctacataggggcctctgatttgttgtctaatttttattcatttttaacctactaggaacacaatgactgtagaattttaggtgcaagtgggccctttaagtcattctgagcagtaggggtgagctgatccattctgagcagcagggcttattacagtccagccattcct

SEQ ID NO: 2 - DNA, 463 bp, Homo sapiens
agaagaaaacagtgactggtcccaagtaagtctgaaacccaacaggaggaaacaacatgaaatgttacggcttgagaataatcattggctcaatgttctgccctccaggcccactaaggtgatagtgccaccttcaggacacaatgtggtagcagccctgccctgtggctttgggtggccttgcccctgaggcagccatgtgcttgtgccttgctcctgcctccacagtagctctgtgcctgggtcatgcatctgaggctctcctggaaaccaccactggtgactctactggtctggactcatgggcctagtgggggccctctgtagtggcccttccccaacagtgattctccgtctcagccccatggctctcttggggcatccttttaattctgggggaaggcagccatgcccccacatctttctactcaagggcccgtgatgggatgggtagctgtgatga

SEQ ID NO: 3 - DNA, 377 bp, Homo sapiens
tatactatacataaaacaattagagaacaactaagtgctaaattaagttttctggcaatggtttctgattatatatttgtttgatttttaaggtatacatgcatgtagtttcagagttagaaggcaaagtagttctataaaccttgtaacaaaaatagcaattcttgaggcctgcatcatcttaggttcagcttttcagaggcaacactttaaattgtttcagctggtaatgttctaggactgtacctccatatctctataacacagatgtatggtttttttttattaggcattatccatggacttttcattatgaaagatgaagatttctgccctaaccccacacccctactccccaccacacacaattgtcttct

SEQ ID NO: 4 - DNA, 588 bp, Homo sapiens
ccggccttcccgtgccctcacagtcctcctcctcagctgtttcagctaaagtcccaggattaatgcttattggctggcttgggcctgaactgaactccctgaactgaggctagcaggatgaaatgctctaatcagccagatgtgagtcattcaccccctcctggagcctggggctggggacctgtgggtgtcaaccctgccaagtgacctggacagaacacagaggagcagaaactccccagagggaaactgaggaggtgggggtggagggacacagagccagcagggccaccggaaggaggcccttgcatttctgcacatccacccagccaggaggagacagctaggcccaggggttgcggcagtgcgtgcaaggcgttttcttgcaggagaggctggtgtttacaggggacaggaaatgtgggtgaactcagccgttttctttgcgggggcagaatgtacaggctgatacagtgaccgcagaagctgctgggtccccctgtggggttttgtggcagtgaagacccccgtctgcccccctgcacagctccttgggcttccaaatattttgtctgtgctgacagcttt

SEQ ID NO: 5 - DNA, 186 bp, Homo sapiens
cctttcatcaattcttgaaaattcttaggctttatgtttcaaatattgcctcttctctttttcttttacttttgggaaactcccattatgtatatgatggactttcttattctgtctttcctatctttttccatattttccactttttatattggtctcctttttcttagaatttctcaaatctct

SEQ ID NO: 6 - DNA, 269 bp, Homo sapiens
tctgtgtgtttacccaggggactgccgcatggcccatgccgagcagaaactgatggacgaccttctgaacaaaacctgttacaacaacctggatccgcccagccaccagctcctcacagctcatctccatccagacggcgctctccctggcccagtgcatcagcgtggtaggtgcagagggtacctgtggctcaggctcaggtgaagaggcagctcatgcccaagccctaagcagtcaatgtccagaggaatgaaatgactagagttga

SEQ ID NO: 7 - DNA, 637 bp, Homo sapiens
cccttgtgattcagacatctgccctgggacccacagtaggttccgagggagacgttcagcctgggctggcctggggatagcctaaagtgggggtgccatgggaggggctgagtgcttggcagcttagaagggtcctggggaaaagcttccagggcagcgtggcaaccaggttatgtggtagggagaggggatcactacacccccacagctaagggcaagtctagagaggggtaagagagaggaggggcccagataggcagtacttgttatagagtacgatgtctggccgccacacaagactgctggggatgcggatggcatccaggccaccataggcattggggtcccatcgtaggtaggcatctgtccactcctgccgtatccacagatacagggtcagcacctggttccgttcatcctagtgggggcagggaatggcagagatgtggacatgtatatgcatatcctgccctgtctgtgcacactcccctgcagggctctggtcagcacccacaaacctgacttgtccataccgtccagttcccacccagacctgaccttgccatgtgaccttagtgggctcttctcttttctgcccgtttcctcagcaggaatatggggtgagaatccctgctta

SEQ ID NO: 8 - DNA, 640 bp, Homo sapiens
cctcttagtttgtaatcagccaccctatttttttttttttccaaaagcaaattgttctttgcaagaacaattctattgacttcaaattactcttgctataggtcctttcgaagtatcgtcatgacatgtacacacagacttgagggaaaaaagtgcttttctgaaaaagtaatgattgaaatttttattttaaatgattccttagattgaattcactttagattaacagattttcctgcccaattgattttctggcatccatgcagtgatccagcagagataaaatgggggttcatttagtccatggctccaaggaaaagtgagagcctggcaaagagagccagcaaagcttctttcttgcctgctcggttggagcaggacaactggagccggtcagctgctgaccagatgctgccttcaattaatattccaaccctcaaagacatttatcgcttactctcgaaagcagagcagctgagtaataaagggaaccactaaagctgtttttttttcaagagcatttataatggctaaattgcttgaaataaattagcacgaaaaataaacatagtttgtacagtatctgtaaaacaaatttccaatcttgggaaaatagagcgacaaagtgggagcttgcatt

SEQ ID NO: 9 - DNA, 573 bp, Homo sapiens
caagtgcaggccaatatattggtgtggtcctaactccaagtggttttaggccatatttaatatctgtctgccaaaaggctatcaaggggtacttttctggtgacaccttgttaaatcaagaatggggggataggctgtggttattagggtcacaaatgggagtgggaggatcaaggtttaaagaagaatggaaagggtgggagaggccgacaggacaagctcaccgtcactcacctctgtccctcactgccctgatgcaggtatgggacaatcctttcattaattggaacccaaaagagtgtgttggcatcaataaactcacagtattagctgaaaacctgtggctcccagacatcttcatcgtggaatcgtgcgtatgcaggctggggaagccagcgtgaaacctcatctgccgagaacagcctagggtcagcacagggcatggggccaccgaaagattcagacaggcacacagtctcaacgaactgacttccacacatcactacgagtagaagaggcgagagagtgacattaaagaaagagcccagggccaggcgcggtggctcacgcctg

SEQ ID NO: 10 - DNA, 716 bp, Homo sapiens
caattatatacctgtgtctttaatccctgagagcagaatgatgaatatttgagccccagtatatcatatatacatgtaattaatttttaaaaggtagttcaatattcaaatttattgcaaagtggccaagaacagtgcaagtgttgacgacttataaatagaactacattgactatttacattaggttcttgaggattgaaataacattcttctgtttttcctaataaatgacaggcttatatacatagacttgagttaaaaattgaccaatattaactgccatgagcccgtggtgaacaaattattgctgtcatctcaaacacaataattaatagattaattactaggatttaccaaaatggctttttgaagatctatttttaatgttcttttctgttaaaagcagcttacacaagtttcctaaatctatactgccactaatgatagtaccacagcatcttagtataaaatttctggagtttgaatgtttgccccctccaaaattcacgttgaaatttaattgtcattgtaaaagtattaagatatgagacctttaagaggtgattaggccacccagtattatgggtggaattaatgccattatgaaagaatgaatttggttccctttttcctctgtgtcctttggccatgtaatgagacaacaagaaagcccttgtcagatgtcaccattctttatattggactttccagcctt

SEQ ID NO: 11 - DNA, 630 bp, Homo sapiens
cttcttattcttggactttataaatatttgaaccatcacatgtataagttcaggccatatgtaaataagacattgtacatacttgattggtttattattgcctattgctttctccctatcaattcccccaaaatcagtgttatgcagatttactgtattaaactacaattccattccttcatcctttatagccatataaattatatttctgagagtagctaatatatgctgtgattccttaaagtcaatataccacagtctgatccaatctaggcagaaagatatagtgggtcaaatttggaatttaaaacatagggcttcttcaggtttatttaagcttgctaaaaaatcaaagcctaccaagctagttagtctttctgtgtcacacttgctaccaatggaagttctcccttttcagaagtaatagaggtccacacagttgtctggaagaaaattgatcttgcaagtacatcatgtctattcaacaccaaatttactaggttcaacatggagcattcaatcagagtgtgtgtctataagaaccaagctcacgttcatgtgattattctggttgggccaatgagttgcttggggctctgtaggaaagatttacagcaaagtagtaaggct

SEQ ID NO: 12 - DNA, 619 bp, Homo sapiens
atcagctgaaggatcaaagtcacaattactagctgtgagtgtgccaagctaaccatttagcacccatgccacaagcatgctctgtgctactcagcatcatgtacacattctcagaagtgacacaagttgacatcagaagtgttttgtatttcagatttagggatttttcattatagttatcagttgagcatctcaaatcctgaaaatccaaaacactccaatgagcatttcctttgagtgtcacattggtactcaaagaatttcagattttggagcattttggattttcaggtttggcatgtacattagtccacgttcacactgttacaaagacatacccaagactgggtaatttataaagaaaagaggtttaaatgactcacagttccacatggctggtgaggccacaggaaacttacaatcatggcgaaaagcacctcttcacagggcagcaggagacagaagggtgaggagcaaagggggaggagccccttataaaaccatcagatctcctgagaactccctcgttatcacaagaacagcatgggggaaatcacccccatgatcccatctcctaggattctactggatccagcactgtccaatagatttttttttt

SEQ ID NO: 13 - DNA, 448 bp, Homo sapiens
gaccatttaggtggctatggtcataatcatgaaagcttggacacagtggtggtggtgcaggtgatgaggtttggagcaaaggatgacgtgatctgactgaggcttaataggatcattctggtttctggggatgagaaagtaaaatttgtagatattttgaagcattttctgttggcctgaatggcaggagtatgtgtggaaaaggaagaaggaatccatagacttgctatttgagtttagaaaaggttttggcctcatcaaggtatactcggtcactgggcgtgtgaaaaaagatggccgagggagaattcctagaaggggaaaatagggagggaggacatgggaggataacagactcctaaatacatgtggttgagttcattggttgtgcatatggaaattacccctacctcaaaccatcacacaaatgatgaatttaagatatcag

SEQ ID NO: 14 - DNA, 547 bp, Homo sapiens
actcctgaaatcctagcccggaccctgagccattcaactcaagcagcccctgagactaacatagggagctgcctggagacttcccacagtattcattctgagaggaagctcacacagggtcctagacagctcctaaatcctaagcagctacaggaaggcaccattttgagaacacagcccctatcatactgtattctgctggagggcccaatagcccctgtatcttcacatccctggagccccattgacattctccacctttattcaccaccgcagctggctctgctgccaaggccaaaatgcaagccattgtcagtaacccagctgcctccagtagcagggccactgtgcatttaaaggcatcccaaaaaaaggctatctcacttatagcagccacctgaggccaaaatgtgtgctccccagccaccttactgttgccactgaaagcaaccctgccctccctagcagcagggtcctggcacagctgctgctgctcccacccaggcattctgccaatggcctgggatcactacattccgggctacca

SEQ ID NO: 15 - DNA, 700 bp, Homo sapiens
ctgggcaagctttaaagtttgggattttccactgtcctttccggtgcgagcatttattgaattttgcagtagtctccataatttactgaggagctacaggaggagaaacagaaaacagttaggatatgccatgctttccaagaggaactggcaactgcagtgaggatgcatttaaacaaaccagtgtgaggatagatctctctacgttatgcagatccactccatttctaaaagcaagttgaacagcaaatttcagttgatgggaacctatatttgattattttaaaataggaaaacagtgattacatttataacagtgtaaaattggtaatgtattatttataattattataatcatgtgtttccaatccaccaaaagaatatgtaccaatttggccaactatcactaaaatactcttaactctatagtaaatcaacaaggttttattcaagctaattacaaccccccccctttttttttttttagcactttgcaaactttaggactgtgcttgtgtgtggtatacacattgaaataaacagggtaatttattgtattctaacaatggctccttctctcctcctccctatggaggaatccccggcaaggaggagtgaaagggtctatgagtgctgcaaagagccctaccccgatgtcacctttcacagtgaccatgcgccgcaggacgctctactatgg

SEQ ID NO: 16 - DNA, 567 bp, Homo sapiens
cagcgcatcgtcaggtccccccgcgcccccgctgctcaccgatgagcggcacgctctcggccggtggcatgctctcggccagcagcaactgggaagacggtgagcgccagcagcacggtgacgcccagcgacaccttctcgcctgagtcggcaggcaggtggaaggcgagcggcgcaagcagcgagatgagcacgcagggcagcagcaggttgcacacgtagggcggcggcgcggcggcgcagcagcagcgtgaaggtgacgtcggggtagggctcggagcagcagccgtaggtgagcacgcgccgccgcgccggcatgcccagcacgcgccactccacgttctccacgaagtccgccaggctggctgcagcgccgcgcggccgcacatccagttggtgcccgccgtgagtccaggagccgaacgtcaggccgcagtgctgggcgtcgaacgggaaggctgctacatccacgcggcacgagctgcgcgtgatggccggcgcgttcccagcgcacggcgccatcgtggcgcaggaccacgttggtgctggcggaacctggaggctgcg

SEQ ID NO: 17 - DNA, 507 bp, Homo sapiens
cctcccctagcacttgacctttattaactcaggtaagcatcaccacaaacctaggaagtaggtcctctgggtatcccatttgtacaaaaagggattcgtatcttgccccagctcatgcccgtcgttatttgagagcgggactgtcctggattgtgtatgagtgcagcctccagcagtgacgggagcaattagagagcagtagcttctgatgacccacgtgtaggaatgaaggatggggagaactcggcccttacctccttcctgcttccatccatggggcttggagggtctggagagcttcatggtgggcttatttccatttgtgcagaggtggctgggaagctcaggaaccacaggcttttgttttgagtcaattggctttctctctctcttgcagggaagtactactggccactatgaccatggtcacattctcaacagcactcaccatccttatcatgaacctgcattactgtggtcccagtgtccgcccagtgccagcctggg

SEQ ID NO: 18 - DNA, 446 bp, Homo sapiens
agggccggctggctctcaagctgttccgtgacctctttgccaactacacaagtgccctgagacctgtggcagacacagaccagactctgaatgtgaccctggaggtgacactgtcccagatcatcgacatggtgcgttgtggtggtggtacagctgtggagtcttacctgtcacagtgtcaagaaatgaaggggtgagagactgggattattctccatggaatttcttttctgtaaatgttaatattaacaaaggtagcagttacaaactgttgggtactgactgttgggtactgagtattgggtgcctacctcgtgcccaatattttgttcacctgaacttactgaatccctgctaagcaggggattctcaccccatattcctgctgaggaaacgggcagaaaagagaagagcccactaaggtcacatggcaaggtcaggtctgg

SEQ ID NO: 19 - protein, 588 aa, Homo sapiens
CAGTGGGATTTAGAATCCCTGGGTGAAAGTCTGGACTCTTGTGGCTTATTTGGGCCCCTCTAGCATTTGTGGAGAGGCAGGCAGACTCCAGGTCCTTGAAAAGGGGAGGGTGGAGGAGAAATTTGTCAGCCTGGCGCCAGAAGATAGTACCAGTTCACTCCATGGCCTTTACCTCATGTGTCCCTGCAGGCAGGCCAGGGAGGAACTAGAGCCACAGCTAGAGCAAGAGAAGGCAGACACCAGGAGGACACTCATAAGGACAGGGCCCCAGCCCTGGGAGTGGAGGGTGTGAGCAGAGGCCCTGGGACTAGGGCCTGGGATGGACAACCCTCCTTACTGACCCTCCAGAGTGCCTGGGAGCTGAGGGCCGGCTGGCTCTCAAGCTGTTCCGTGACCTCTTTGCCAACTACACAAGTGCCCTGAGACCTGTGGCAGACACAGACCAGACTCTTGAATGTGACCCTGGGAGGTGACACTGTCCCAGATCATTCGACATGGTGCGTTGTGGTGGGTGGTACAGCTGTGGAGTCTTACCTGTCACAGTGTCAAGAAATGAAAGGGGTGAGAGACTGGGATTATTCTCCATGG

SEQ ID NO: 20 - DNA, 525 bp, Homo sapiens
ttagtgacgcccattatccaaatcttctaactattcaaaaagggaatcctacaaaaataaataatgcagtattgttttattgagttatacctatatgccccacatactccaccaaagatttattattgatctatccagtctcacccatttctctatttttctatttgtctaataaagcagtcctcatttgttcctttgtctatctgccatccgtccgtccttccttccttctttccacagacttccttctacatccctgcctctgtcttccccatcatcagtacatgacatccctatctacccattgtttagacatcatccctacactcactgattctacattttaattatttctcaaattcatttacctggtgatttttctccataagcaccctaatcctgacctatgattcatctctatactgagagtctcttcatattgttttcatactatttattacaacaataattataattagtaactgtgtttaatgtctgtgtaccactaaactataccacagctcc

SEQ ID NO: 21 - DNA, 633 bp, Homo sapiens
tttacaataagcaaaggtgacagcaacccaagtgtccactgacaggatgaacgggtaaacaaaacatggtatatacatacaatgggaatattatttagccttaaaaaggaaggaaattccgacacatgctacaatattatgttaaatcagcaagtcacaaaagaacaaatactgtatgatttcatttatattaagtacttagggtagccaaattcatagacacacaaggtagcatggtggttgccaggagctgggggcagggggaaacgggagttatcgtttaatagataggaagtttcagtttgggaagatggaaaaagttatggagatgtatggtggtgacatttgcacaacaatataaatatacgtaatgccactaagctgtatacttaaggatggttaaaatagtaagtttaatgttatatatatttaaccacagtttttaaaaatccaactagagttcattgattctttaagtacttctgtactttctgaaataaaaagatgttcaagcccttcttatattttccttgccctactcctgctgctagccatttcttcaagaattcttagttccttttagtagactcatatttagaaaccaagatctggacactagacatgctcattgct

SEQ ID NO: 22 - DNA, 505 bp, Homo sapiens
cttctgcctcttttttacatatttctatttttaaagtttgtgagtcaaagaagttttcacatatcctaaatgcttattggaatatgtataattacatttggaatgttgatgcatacacttctgtttttttgtttttcttagaggaaaaggtgtattttcctccattgatttgtgtaaattttttttcaaaagcttaataagtattttattttgtttctgttcatttttatggcattaggacaatttaataatattcccagtgtaagaaaaacctcttctttcagtatagcaaaatccaaataattgaaaagattttatttgttttcatgtggagaaagaggtgagtcctccgattttatgaatctctttagtgcagtaggacattaaatttgctccccttttctacttcttgccatcactaaccaattgccaaatgacacatcttcctgttttgtttccccagaagctatctgcatttttaagagcatctgtatttgtatctagc

SEQ ID NO: 23 - DNA, 636 bp, Homo sapiens
cccacaagtgtcaaaggaaaaacggaataagaattcattcaataaaacaggccttaaagatgaatttttttaaaaaaaggtagaataatgttaacatggaaagtgaaatagagagacaaaattgagaactaggcaaaacattacagagttaccaagttaaccataaagggaaaggaatgtagtaatggcaaagagaaaatccttgagataattactctgaattcagaaaaaaaaaaaaggagacaaggaataatcacagagttgatgaaaaagatggaaggcagagatgatacaacataggaataattggtttcctttaattagggacccatactaatggaacagaaataagtttacagaaaacttttccctaaaggaaggaagaaataaactatatattgaaatgacgtgtggtatataagaaaaaaactgattgataaagaagaatttacatggaaacctcacttcaaataaaatctgaagaccttcaattgcctcaaagcccaaggtgacacatatgtccattgcctctgtgacttcatctcatatttattcttggaagaactcactcttcactggccatgcttatcttccttgctgtcactcaatatgtcagtgacaataatgcccatggtc

SEQ ID NO: 24 - DNA, 578 bp, Homo sapiens
atagtcaaaccaaaactgtatatgtactttaactcccatggtttcccattcattaagcagaaattgaatgtggaaagggccaaatattctatttttctccacctaccttcctttttcagggtgattttctttgagtttggagaatgggttctggaaactgtaaaaggcagaaaataatatttattttactagtgctgttctgtccttcattggttcccctagctaagattgactgtcattgatatttatgaagttggcatccaaatgctgactccattgtgcaaaaaacagagagttttaagagaacttgtaggatagaaattcactttagtttggactctctaaattctctcctcttaactcttgcctgcaatagtacaccacaattttcccccttcatcaggtgacctctttgcataaaatatttaaaagaagggcttatgcttagcaagagtccacgtggcctactttacatacaaaaaactcaaagattcttattttgtcaattctcttttccttcaaaaaaaaaataatgagaggaaaagaaatctggcacctcattggcagagatcacctgc

SEQ ID NO: 25 - DNA, 727 bp, Homo sapiens
attatgacagttgatcctcataacaacctctggagctacatactgggtgctgttgttattctcactttacagatgagtaaactgaaggtaagaaaagttgagtgccccgcccagggttgcaaagcgaggaagtggtggagctgggattgggtgtgccacagtctctttctttgggcagactgaacatgcctaggctcctaatgattctgctatcttccttcctttccctgagccccgggctgtgcaacctgtggccagctttcctgacggggtacatctcaaccctaccccatccctgaaagaaggggcaacacgcaacacccattcactccctcccaaatgctggcactgtgctgggggctgggctgtgatggtgacggtccctgccctcgcaaaggatactgtgtatgggcactgcgctgtgatgtgttggctgtcataggcacacgcaggagggagacagggctgaggaagtggagagagtgagacaggcaaagggaagcgggaagagtgttccaggtagagggagagtctgagcagaggcccagagactgaagagacaggcacatctgagaagctgaagggagtccagtgggtacgtgcatcaggacgcatgctgggaggtccctggggtggggttatgaaaggtgccaaagagactgaatggccacacagagcacactgaagccactacagttgcatattcagaaatgcctgagttcctgga

SEQ ID NO: 26 - DNA, 721 bp, Homo sapiens
caaatacattcagaaaactctgtacttaattcacctctttaggactcataatacatattagcacagtcaagacactgagaaagtcctgcagtaaataaatgtggtttatgttatttaatccagtgttttaatattagggcacttttgctaaattactgtgtggtaacgaataacctcaaaatcccagtggcttataaccacaaaggttgatttgttgctcatatttcgtgtcagctgtgctttggctctgctccagatgtcttcttcatttcagatgtaggctaaaggtgcagccttttttcaggaatatgccattcttatgataaagggaaaagagcaaaagccatgccagacaatgtctcctaaagtgtcttgcccaaatgtgtcatgtaccgtgtctctcacattccattgtccaaagcaaatcacatggacaaggccaatgtcactaaaatggaaagtcacagagcctcccacagtgcagtgctgccagtcacatggaaatgcactgtatgtatataatcctcttagaggaaacgaacaataatgtaataatgaaatctgccacaaaatacacttatttttacaccaaatctttttttaatttaattaccatatgattcagcaattttactcttaagtatatattcaaaagaactgtagacaagcattcaaatgaaaacttgtaatgaatgttcatagcagcactattcatagtagt

SEQ ID NO: 27 - DNA, 680 bp, Homo sapiens
atggaatgaatgaatgaatgcattgaaagcctacttacctaaaatctcctatatattcaaatgattaatcaaagatctttcattcaacaaaatgaactgagtgcatttagaaggcattgtggggtgaaggagatgtggccccttcctctctggagcttagagtctgtctccaccattgaatctgaaaagctagccaaatacatgagtaaaaaaattaaaatccaaatcttttaccaatataacatcggatgacatggctgtaatgatcaaataattacctgattctttccgattcggttttaaatgttaaacattcagtgatggttaacatactcgctgatgtgaaagggtgggggctgactcattactggggctaggacaagggcaaatcgtggctcagaactgtcattcagagcctcttgtttgtcctctgtagtcagctcagtcacagtaaggtatgtggtttctctcaacatgtcattcttgttttatgtactcaaatgcttccttctcattgtcaacatctgctctgaactttaagtcaggcccacttgtttgtagaatagctcattgacataaagcaaataacacatcccagccagtcaaatccaagaaactcagctttaaaaacacatttgtattaaagaatttcactgcaaatccattcattatgtttacct

SEQ ID NO: 28 - protein, 331 aa, Homo sapiens
CTCTCTTATGCTCTCCAGCAAAATAACTTCAGTGACTTTATCAGAAATGGGGTTTTAGACAGGATGTTTCTTTGGTTAGATTTGGTATCATGTGTCTTAGGTATTTATATCTTTATCCCTTAACCATACACATACTTTACTTGGGGTAACCTTAGTAAATAAGATCTTCAATTAAGCTTAGAACTTTGTAGGATATTAGAAAGCCAGAGTCCATATCTGTTTGTGGGGACAACTCAGACATCCCATCTTCCATTGACTATATTTTTGAGTGACTTTTTCGTAATTAGACTCTCTACCTTCAAATTCAGCTTCTGTGGGATCATTGATTAAA

SEQ ID NO: 29 - DNA, 610 bp, Homo sapiens
agctgagcagagtctatgcaggcccattggctgcctagccagtggtgatcccgctcccaccctcatttcttctttgttaagaaaaccatgacctcattaaatattggacacctataaacctcagggaccttggtgcagcctccccgccacgtattggtgagtctaagtcaactctggtcatttcattcctctggacattgattgcttgaggcttgggcatgagctgcctcttcatctgagcctgagccacaggtgccctctgcacttaccacactgatgcactgcgccagggagagctctgtctcgatggagatgagctgtgaggagctggtggctgggcagatcaggttgttgtaacgggttttgttcagaaggtcgtccatcagcttctgctcagcatgagccatgcggcagtcccctgggtaaacacacagacatgctgggcccttgtgcagctgtcccacactgcagatgacagctacaaagcaggagccaagagggccaggggagcacaggcaccccgggggccggctgaagcagtgaaggtgctggcggaccaggctctccctggggacttcaaatgacattcatgacagagctcagctacttt

SEQ ID NO: 30 - DNA, 614 bp, Homo sapiens
tctgcaggcccattggctgcctagccagtggtgatctcgctcccaccctcatttcttctttgttaacaaaaccatgacctcattaaatactggacacctataaacctcatggaccctcctccagcctccccaccgtgtaccggtgagtctaagtcaactctagtcatttcattcctctggacattgactgcttagggcttgggcatgagctgcctcttcacctgagcctgagccacaggtaccctctgcacctaccacgctgatgcactgggccagggagagcgccgtctggatggagatgagctgtgaggagctggtggctgggcggatcaggttgttgtaacaggttttgttcagaaggtcgtccatcagtttctgctcggcatgggccatgcggcagtcccctgggtaaacacacagacatgctgggcccttgtgcagctgtctcccactgcagctgacagctatgaagcaggagctgagagggccagggagcacagacaccctgagagctggctgaagcagtgaaggtgctggccggcctggctttccctggggacttcaaatgacattcacgacagagctcagctacctcctccccatgccatacctct

SEQ ID NO: 31 - DNA, 198 bp, Homo sapiens
ctgtgaggagctggtggctgggcggatcaggttgttgtaacaggttttgttcaggaagtcgtccatcagtttctgctcggcatgggccatgcggcagtcccctgggtaaacacacagacatgctgggcccttgtgcagctgtctcccactgcagcttgacagctatgaaagcaggagctgagagggccagggagcaca

SEQ ID NO: 32 - DNA, 540 bp, Homo sapiens
agctccatctcgatggagatgagctgtgaggagctggcggctgggcgggatcaggttgtggtaacgggttttgttcagaaggtcgtccatcagcttctgctcggcagggccatgcggcagaaccctgcgtaaacacacaggacctgcttggtccttgtgcagctgtcccccactgcagctgacagctatgaagcaggagctgagagggccagggagcacagacaccctgagagctggctgaagcagtgaaggggctggccggcctggctctccctggggacttcaaatgacattcatgacagagctcagctacctcctcccatgccatacctcttcctcctcctcctccctcaatcaatgaacagcatcccacgctctacacatctgatacaaaactgggtatctcttcctgacccctcccttggttcatataagtggccaccaagtcctgtctgtcctcccatctccacggctacagccatgtccctgcctcccccgccctgcccaccttctattctctccacctgcaccctgcccctg

SEQ ID NO: 33 - DNA, 334 bp, Homo sapiens
agacacccagttttgtatcagatgtgtagagcgtgggatgctgttcattgatcgaaggaggaggaggaggaagaggtgtggcatgggcggaagtagctgagctctgtcatgaatgtcatttgaagtccccagggagagcctggtccgccagcaccttcactgcttcagccggcccccggggtgcctgtgctccctggccctcttggctcctgctttgtagctgtcatctgcagtgtgggacagctgcacaagggcccagcatgtctgtgtgtttacccaggggactgccgcatggctcatgctgagcagaagctgatggacgaccttctgaaca

SEQ ID NO: 34 - DNA, 680 bp, Homo sapiens
tcttatttttccaatgtagtttctagaaccgttagcacagaaagttataaacattgtataattattcatctaaatgaattgtaataataactacaaaaaattatgtctactggctgtaacataacttagtaattattctgtttgtatgtacttaggtagcttccagaggtttatggctaaatgatctctaataattattcttattttcaaatttaaatgtcaattgctgaatatatacatacaataaaggctttataactatgtgtattagtttgctaggaatgtcataacaaaataccatagactatgtggtttaaacagcagaaatgcattttctcacagcttcaaaaaggctctaagtctggtatcaaggtgttagcaaatttggtttttcctaaggtctatcttcttttctttcagatggctgccttcttcctgtgtcctcacatgggcttttctctgtgcatatgcatcctgtgtctatgtccaaattttctttttaaaataatgacccagtcatactgaatgaaggtccactcatatgatttcatctaagcttaattaccactttagaggccctatttctaaatatggtcatattctgtggaactgagaattagctcttcaacatatgaattttgggggacaaaattcagcatatatttcctgatacatagagc

SEQ ID NO: 35 - DNA, 619 bp, Homo sapiens
ttcaatctgcaatgtccttgcactgaccagggctccatttcctttatcagaggctatgatggaaatgatgtgggagttcacctggctgagacggaatgactctgtgcatgggctggaaaccctgtggcttgcttagtacaccatacaatggtatttcaccttggacaccagattgcagcaggagacaggtaactcatgtgacaattttttttttttaatttttaccattgttttcgtagatattcctaggccagttctaagagtttgtttcttgggagattagtgctggaggccagaagtctgagatcaaggttggttttttctgaggcctctctccttggcttggaaacagccgttttctcggtgtcttcacatggtcttttgctctgtacctgtccaaatttccttttcttataaggacatcacttgtataagataagggttttcccctcattttaacttaattacctctttaaaggccctatctccaaacacagttacattccgaggtactgcaggtcagggcttcagcacatgaattttgggcaaggatggagagggttggaaacaatacaattcaccccgtaacaccagatctgactcctctcactagcctcct

SEQ ID NO: 36 - DNA, 605 bp, Homo sapiens
attgcctgctctggaagcatgcaaagtggaccaaattcagtccaaaggtctgggagtaaatttagctctgccacttacttgccttgtgaccttggacaatgatcatctataaaggagtgatgagaaatagtactacttctttgttatatgttgtgtgtgtgtgttttgcgtgtgcgcgcatgtgtgggtgcgcgtatttaaaaagctaagaaatgcaaaagggtcaaaagcgctaagcctgggctcaagaggtgctcagggaaagctgattgtcagtcaaaaagtcaaacctgcacgtttcctaccaccacttgctggtagcggtagcgggcaatgactcttcggggtctcctgtgtcgcctaggctggcgccgaggtcctcgactgtagaaaagatagttgatgtagacatactccagcaaggacaggaacacaaagaacaagcacacgaggatatagatatcaatggccttgatacaggaaatgttggggagcttatcccgcagatgtgagtcgatggtggtcaggatgagcattgaagttaagcctgtaagcaacacagtacagacttagtctcctctgatggctaacgttcttggcaacct

SEQ ID NO: 37 - DNA, 667 bp, Homo sapiens
aaacattcaaactgtatcagaggccaaggcagttccaggctgagtgaacagcagtgtcaaatactgctcaggtcagagctggtgtggccagtgaactgggaaatttaacatcagagggggcaatcttgactttcctcaaagcattctcagtggagtggtaggagtaggagtgaggtccagaagatttggggatgagtgagtggctgagatgggaaaacagcaagtgtagaaaactcatacaagtttggttgtgaagtgacagaaagagtagctagagaagtggaaggattttccttagctggtagagatccagggatgctccattgcttatgaggggacaggaaagaggggagggttgaagatatgggatgaatgacagggaagaaagcattcccaaacacagaggaggtcccccaaaatggatcctgatacaggtaagtggaaaggtttgtggcagaatgttgagaaaccatccattcaatggcttctgtttagtctctgatatgaaagacaaagtcacctgcccagatggatgaaaagatagtgggatagaaaactggaaaaaaaacaaaaaagggaaaaaggtttgaaatagcctttgagaagcatgaagagagagctggaggcttgctgaactctgctgagagccagtggaagctggagactg

SEQ ID NO: 38 - DNA, 518 bp, Homo sapiens
cttttaaacacagttgggacactaccattaaagaggaatcttcatcactaaaagtaaggtaattttgttagaaaatgcaaatcctaacacaaaaaatcggatcaaaggtaaatcacaaataatgtttgaggtacaaagaatctaccactgtgggaaaattcaggccataataaaccactctttacacaggggatccaatgggagacatttgaaaaacagaaatacacttttcttggtgagcaatgttaggtactccagtttcatcttaactttgtctttggttatgggtctcaagcgtccctatttctgtaaacaaacacataaatattcaaaagagtatctctaagtaagttgaggtttataaaatagaaatttttctttttaacataccgaggctttattttttagctttctgtctttagtagcagtctttcctttttggttgctggtaaaataatgcaaggttccatattccatcaagggctgcaaaaacaaaaatgaaacaaacagaaacaaag

SEQ ID NO: 39 - DNA, 617 bp, Homo sapiens
ctctatgtagccccaactaaacatgtctgtgggctagattagccctttggccagctggccaccagttgaccatttctgtagacaagattctcagaaaggcaaccacagcctcaacttttacaggattattttctacctaaagaggcatgtgcataaatggcaggatgcccagcacacctcattttactgtgtttcactttattgtacttcacaaatattgcattttttaacaaatggaaggtttctggcaaccctgtgtcaagcaaatctatcagtgccatttgtccaacagcatgcgctcccttcctgtctctgggtcacattttggtaatttttgcgacatttcacagtttctcattattattatatctgttatggtgatctgtgatcagtgatctttgatattcctattctaattgtttcagggagccacaaactgtgcccatataagatggaaaacttccaataaatgctgtgtgtgttctgactgctcatcactgattggctgttccctcatctctcttcttctcctagggcctccctattccctgagagacatcaatactgaaattaggccaatcaataaccctacaatggcctctatgtgttcaagtgaa

SEQ ID NO: 40 - DNA, 670 bp, Homo sapiens
tccagctcagaaactaccagccttcatcaacatgctgagcttaggggcatggatatgtggagagcaggagcctcagtggtgcccttgtgtccccagtcctggctggacactcgcctggcctggaacactagtgcacacccgcggcacgccatcacgctgccctgggagtctctctggacaccaaggctcaccatcctggaggcgtaagtgagacagttcctgccccaggaatctgccatgcatagccctccttttccccatctacaacctagaggctgtctgagtgaatatgaccctcctggcggtccccgccggactagcagtgcaccttcactgcctcgaattcccctcccactgccagaactctgaaagcagctggggttggggttgggatgccagggtctccccccggccccgtccaagaaggggctggggctctggctgtggtgcctttccccacaggctctgggtggactggagggaccagagcccccaggctcgagtagaccaggacggccacgtgaagctcaacctggccctcaccacggagaccaactgcaactttgagctcctccacttcccccgggaccacagcaactgcagcctcagcttctacgctctcagcaacacgggtgctgacagggcaggggctgcagggttgaggagggga

SEQ ID NO: 41 - DNA, 652 bp, Homo sapiens
aggccatggcaacctgagcctctggccttgctgcaaggggccgagccactgcagtcgccatggctgtggagggcagttgctctggggaggacagaagactgatgtgctcggacctctgggattgcagagctgctgcgaatgtttgagtctgtcaccctagagaggggccctgaggctaccgctgagcacagagatgggctgccactcgagtggggggcgcagtgggagagcaggtgctgcccgcctaagcctggggtagactgctctgaacacagatctgggagttcgccttctgtctgcctttgccccttccccttgccccgcaccctgcccctgcaccacagacctgggagttcccctcccccaccttcctcctcccctcctcaaccctgcagcccctgccctgtcagcacccgtgttgctgagagcgtagaagctgaggctgcagttgctgtggtcccgggggaagtggaggagctcaaagttgcagttggtctccgtggtgagggccaggttgagcttcacgtggccgtcctggtctactcgagcctgggggctctggtccctccagtccacccagagcctgtggggaaaggcaccacagccagagccccagccccttcttggacggggccggggggagaccctggca

SEQ ID NO: 42 - DNA, 680 bp, Homo sapiens
aattatagaaaatccaaatatcctggctggggtgagagtctgtaagctagccagagaaaacagctaaggctaagaaaataaaatataggagaaaattctagaaaatccagatatcctggctggggtgagagtctgtaagctagccagagaaaagagctgaggcgaagacaataaaatataggagaaaattctagaaaaatgaaaattggtttattgtcccagatctgtacccttctccccctctgattgttcacttgattttagatggtgaatgacaaatattggtgaagaaaatcattccatgaaacactggtaaccatttgtccgaaacgccttcatggcagcactgccgtggctcagtacattgcacctgcacttccaaagtgaaggtgactgttacctgaaacccatgtgcctggcacacatgaccagccttggacacaagaggcctttgatcagaaactgggaggcactcccacattcccacaatgaaattccgtgggtgcctgtaccctgagttcatccaacacatggttactgatcatgtagggtgtaccaggctatgtcagacgttagagacaccatgaagagcaaacagttagcttatggggagtgcctaacgcacacctgccatttacatctttgtcctcatgattcttcccactgaaccaatggcactg

SEQ ID NO: 43 - DNA, 559 bp, Homo sapiens
atgtctctttgtttaattagttttgggtggctcaatttttaggactattgttcctgtttttctttcctcagttttaattgccaatttaagctctggacaaaatctgaaaatttacaactggaattttacaagaaggcctcgtattataaagtttgttgcttggtttgtgagacttgggttgtggacagtttgaataaggttttcatagaaaagcatcagtgaaagaaagaaaataaaatatattttaaagtaactttcctccttccaataaaacttctaaaagtcaatacatatgactttttcaaaaacataaaaaaaaatgccagatatagggctcttcacccaaagattaaaataagttttttttaaaacaaacaaacaaacaaacaaaaagaacatatggctgaaataaaagtgcctcttggtagaatatgcaatgaaagtgtaggttgggtccagagaaacagttgtgtgcagacatcaattctcaggagacaatgaggagtgaagcaaacaagattgaatggcggaaagttgaagggtgatactgttgaaatag

SEQ ID NO: 44 - DNA, 648 bp, Homo sapiens
ctccttcatagattactcttttcattacccttgtgccatataacatcttagctgtgtgagaccagggagaaaggtgttggtcacctacccttggcagtaggaagtctttcagatctgatattaattgtgtattcaaatgtcaaggtatcctagtacagaaaatatcagtgggttattctgataaggaaaatactatttgctaattttagaaaagagaatatgctaaaagttacacctcagagggaatatcatttgatatggtgaacaggaaacccaagaagttgtgaattccattcaaaagatgaaactgcttagaagataatgtaaggttctcacccaacatgagcactgcactcaaggccatttctaggatgaaagggtgggatgattatctattattccagccatgaattatttctgtggcctccagaagatgcaactgaattgtagctatgtgtccagaatcggttccttctggtgggttcttggtctcgctgacttcaagaatgaagccgtggaccctcacggtgagtgttgcagttcttaaagatggtgtgtctggagtttgttccttcagatattcagatgtgtcccggagtttctttcttctggtgggttttgtggtcttactgacttcaggagtgaagc

SEQ ID NO: 45 - DNA, 585 bp, Homo sapiens
agcagtggcatgataggttcattccttggagtctattgtgtgtgtttggggccccgtaaaatattagaaagcgatggaaattttagggctccgtataatattgtatttacataaccactcagctctcaactactctcaaagagtacctactgaagatcatgtcttcaacttgctaaggctgatctgggtattagccaactctctgagttgaaggaaacagatgtaaccaggtcatctcatgaaatggagctctattgttcagtagatgaggtagtaagtggagcagacactgctgtttgccttctccctggctaacagaggactgacattgactggattaaaggatagagctaccctgtacttcaggcggctgcattcctccctgccggcaccagtgattgatttaggaatgggtagagggtgcaattctgaccaatgagacgtgggagaagcttgctggggagttggtggggtattttccttttgctttaaaaggggcaaaggaaaggtacattccctttttttttcctttttcatctctggatgtcattgcctggaacttttgcaggcttctgataccatgag

SEQ ID NO: 46 - DNA, 642 bp, Homo sapiens
cccagcagaacataaggttgtggctgggacatgaatgcaccccagggagcactgaactgctctgagctgccgactagggccataggctagctatgtgggcccatattgaggtaggggctgagcagtcccagcggcaccgcccaggctgcctgctctggggtccctgcaaaagccgccgctgagcccacggacttccgggtcgtaagcacgtggggcctgaacatctgcttggctgggtcagctgctatgacaatgcccgggcgatcgtgccctccagcgctgcctgcatgccgaggaggaagcgagtccccacgtgaataatcgggctccgccggctcacagcggatgtcagaaggtcaggtcgctctgctcctttcgcctccgtttttcttcctcatggaaactttcttcagctgcagaaaaagctggtccttttctttctgcctggcccacagcttctcctgcaaagtcaaaatttgttccttcggtctcctctggtgacattctctcttccatctcctttctcttccttcgtctgctcttcctccatcttctcgccatcaccttatccgccgcctcctcttccctctcgccccgcagcctgcgctcccgccgggggcgctccggacacactgtctgcgc

SEQ ID NO: 47 - DNA, 657 bp, Homo sapiens
ctttttctgcagctgaagaagtttccatgaggaagaaaaacggaggcgaaaggagcagagcgacctgaccttctgacatccgctgtgagccggcggagcccgattattcacgtggggactcgcttcctcctcggcatgcaggcagcgctggagggcacgatcgcccgggcattgtcatagcagctgacccagccaagcagatgttcaggccccacgtgcttacgacccggaagtccgtgggctcagcggcggcttttgcagggaccccagagcaggcagcctgggcggtgccgctgggactgctcagcccctacctcaatatgggcccacatagctagcctatggccctagtcggcagctcagagcagttcagtgctccctggggtgcattcatgtcccagccacaaccttatgttctgctgggccactttcagcacacccagacagggttcctcttctggtgctgctctgtctttgaaaccgcagatagaccatgctaaccagcacacaggtttccctggtccatcctccctgacccccatgcatgcccaggctctgcatccaggccctagactccttgcctaatcccagctccccgcgaagatgcagccagcaggaacgtctaggttttgcagctaccaaccaaccaggccctca

SEQ ID NO: 48 - DNA, 446 bp, Homo sapiens
tctgaagctgccgtgtatgaacatacatctacacatacacacacacacacacacacacacacacacacacacacacacacacacacacacaccccccgtagtgataaaactatgtaaatgatatttccataattaatacgtttatattatgttacttttaatggatgaatatgtatcgaagccccatttcatttacatacacgtgtatgtatatccttcctcccttccttcattcattatttattaataattttcgtttatttattttcttttcttttggggccggcccgcctggtcttctgtctctgcgctctggtgacctcagcctcccaaatagctgggactacaggggatctcttaagcccgggagggagaggttaacgtgggctgtgatcgcacacttccactccagcttacgtgggctgcggtggggtggggtggggtgg

SEQ ID NO: 49 - DNA, 554 bp, Homo sapiens
tctatagctcccaccctattcacagaagcctggtggatatcttctgaccgtagcactttatagacaacccagtagaagatattgaagatgaggaaagtgaaagggaagacagcccgggagatggtgtcaattctcttggctcagtccacgtagagtttccgcgtggtttctccttcccttagaagaggggctggaggttggggactataaatgccagaaccttccattggacctccatctcttgcctgcaggcagtggcccaagccatagccacggaaatagaaacgactttcttggatgatatcttcctcctggaattacaaggaagaaacggcagaatttgaggtcaaagctcaaaggcagagggatagagaacagaccacccatcaatatctcatagggaatgttatgcagacaaggtgccttgggtacacaggcccattgcatgctttttatggtcacaacactactcatgagatagatgtatgctaagcagctctacgtgctatatatagtgtatgtcatgattccatggcagataggctctaagctac

SEQ ID NO: 50 - DNA, 469 bp, Homo sapiens
tcattaatttattactaagcactagtggaaatctaactttatttacccccatcaacttggcttgtgttaccagaacaagaaggcaaccaaacatgaaatgctttgggaaatgacccactagactgaacgtccaaatcacttttgctgttacatactgtatgacagcggttctcaaacctctgtgtgcagaacacccctgagaacttgttaaaataacggttcctgagccccagcccagagcgtatggttcagtagtttgggggtgaggttggagaatttgcatttgtagtaagttcccaggtgatactgctgctgccactggtcctggactacactttgaggagcctgctgaacacagcacctcagcctctacttgaaggacaaactagcttcttactggattcagtggcaagattaagcccactggttctcaaacacaatccccttgggaacaccagtgctctaccac

SEQ ID NO: 51 - DNA, 445 bp, Homo sapiens
tcagcattttgttgccaagttttctgagacctctggccattaagccttcactgggggtgtggtctgtctctgaagtctactcccattgcaaatggattttgacggtaatggtcaacacgcctgggcaaagaatgggtcatgcccattcttactggaaagatttggaacatttccctgtaaattgtatattatttggatttatttctctaactgaatggacgtttttctatatgttgccaaatcttccagtaatgcttctcattcagtgtaattaaggagattaaaagtgacagcatttttcttgttgaattaatgatgggtttttacattttcacttttcaaaaaatataatcaccactgtgttttgcagaaacaatagtatgataaaatcaaggagaaatacaactagagaagaggcaaaaaaatctcaatattatgattataa

SEQ ID NO: 52 - protein, 60 aa, Homo sapiens
VAIRRRPSLYIINLLVPSSFLVAIDALSFYLPAESENRAPFKITLLLGYNVFLLMMNDLL

SEQ ID NO: 53 - protein, 26 aa, Homo sapiens [sequence omitted]

SEQ ID NO: 54 - protein, 22 aa, Homo sapiens
RTVPPYLYNTDVWFFFIRHYPW

SEQ ID NO: 55 - protein, 33 aa, Homo sapiens
GGRRGSSLPQNPTGGPSSFCGHCISLYILPPQR

SEQ ID NO: 56 - protein, 35 aa, Homo sapiens [sequence omitted]

SEQ ID NO: 57 - protein, 37 aa, Homo sapiens
GDCRMAHAEQKLMDDLLNKTCYNNLDPPSHQLLTAHL

SEQ ID NO: 58 - protein, 52 aa, Homo sapiens
DERNQVLTLYLWIRQEWTDAYLRWDPNAYGGLDAIRIPSSLVWRPDIVLYNK

SEQ ID NO: 59 - protein, 27 aa, Homo sapiens
HFVALFSQDWKFVLQILYKLCLFFVLI

SEQ ID NO: 60 - protein, 40 aa, Homo sapiens
LMQVWDNPFINWNPKECVGINKLTVLAENLWLPDIFIVES

SEQ ID NO: 61 - protein, 37 aa, Homo sapiens
REPNSFFHNGINSTHNTGWPNHLLKVSYLNTFTMTIK

SEQ ID NO: 62 - protein, 52 aa, Homo sapiens
TLIECSMLNLVNLVLNRHDVLARSIFFQTTVWTSITSEKGELPLVASVTQKD

SEQ ID NO: 63 - protein, 42 aa, Homo sapiens
CISDLGIFHYSYQLSISNPENPKHSNEHFLVSHWYSKNFRFW

SEQ ID NO: 64 - protein, 57 aa, Homo sapiens
SSHVLPPYFPLLGILPRPSFFTRPVTEYTLMRPKPFLNSNSKSMDSFFLFHTYSCHS

SEQ ID NO: 65 - protein, 97 aa, Homo sapiens
PETNIGSCLETSHSIHSERKLTQGPRQLLNPKQLQEGTILRTQPLSYCILLEGPIAPVSSHPWSPIDILHLYSPPQLALLPRPKCKPLSVTQLPPVA

SEQ ID NO: 66 - protein, 21 aa, Homo sapiens
PARRSERVYECCKEPYPDVTF

SEQ ID NO: 67 - protein, 85 aa, Homo sapiens
NAPAITRSSCRVDVAAFPFDAQHCGLTFGSWTHGGHQLDVRPRGAAASLADFVENVEWRVLGMPARRRVLTYGCCSEPYPDVTFT

SEQ ID NO: 68 - protein, 42 aa, Homo sapiens
SLSLAGKYYMATMTMVTFSTALTILIMNLHYCGPSVRPVPAW

SEQ ID NO: 69 - protein, 43 aa, Homo sapiens
GRLALKLFRDLFANYTSALRPVADTDQTLNVTLEVTLSQIIDM

SEQ ID NO: 70 - protein, 31 aa, Homo sapiens
AEGRLALKLFRDLFANYTSALRPVADTDQTL

SEQ ID NO: 71 - protein, 43 aa, Homo sapiens
QSHPFLYFSICLIKQSSFVPLSICHPSVLPSFFPQTSFYIPAS

SEQ ID NO: 72 - protein, 69 aa, Homo sapiens [sequence omitted]

SEQ ID NO: 73 - protein, 42 aa, Homo sapiens
FSHILNAYWNMYNYIWNVDAYTSVFLFFLEEKVYFPPLICVN

SEQ ID NO: 74 - protein, 43 aa, Homo sapiens
ETNYSYVVSSLPSIFFINSVIIPCLLFFFSEFRVIISRIFSLP

SEQ ID NO: 75 - protein, 22 aa, Homo sapiens
FFEFGEWVLETVKGRKYLFYCC

SEQ ID NO: 76 - protein, 58 aa, Homo sapiens
EKLSAPPRVAKRGSGGAGIGCATVSFFGQTEHAAPNDSAIFLPFPEPRAVQPVASFPD

SEQ ID NO: 77 - protein, 58 aa, Homo sapiens [sequence omitted]

SEQ ID NO: 78 - protein, 37 aa, Homo sapiens
WICSEILYKCVFKAEFLGFDWLGCVICFMSMSYSTNK

SEQ ID NO: 79 - protein, 23 aa, Homo sapiens
VLDRMFLWLDLVSCVLGIYIFIP

SEQ ID NO: 80 - protein, 54 aa, Homo sapiens
GDCRMAHAEQKLMDDLLNKTRYNNLICPATSSSQLISIETELSLAQCISVVSAE

SEQ ID NO: 81 - protein, 50 aa, Homo sapiens
GDCRMAHAEQKLMDDLLNKTCYNNLIRPATSSSQLISIQTALSLAQCISV

SEQ ID NO: 82 - protein, 34 aa, Homo sapiens
GDCRMAHAEQKLMDDFLNKTCYNNLIRPATSSSQ

SEQ ID NO: 83 - protein, 30 aa, Homo sapiens
AEQKLMDDLLNKTRYHNLIPPSRQLLTAHL

SEQ ID NO: 84 - protein, 18 aa, Homo sapiens
GDCRMAHAEQKLMDDLLN

SEQ ID NO: 85 - protein, 43 aa, Homo sapiens [sequence omitted]

SEQ ID NO: 86 - protein, 39 aa, Homo sapiens
EFTWLRRNDSVHGLETLWLAYTIQWYFTLDTRLQQETGN

SEQ ID NO: 87 - protein, 54 aa, Homo sapiens
  UNSURE (33)..(43): Xaa is any amino acid
GLTSMLILTTIDSHLRDKLPNISCIKAIDIYIXXXXXXXXXXXLEYVYINYLFY

SEQ ID NO: 88 - protein, 42 aa, Homo sapiens
LSFISETKQKPLNGWFLNILPQTFPLTCIRIHFGGPPLCLGM

SEQ ID NO: 89 - protein, 43 aa, Homo sapiens
LFLFVSFLFLQPLMEYGTLHYFTSNQKGKTATKDRKLKNKASV

SEQ ID NO: 90 - protein, 94 aa, Homo sapiens
LASWPPVDHFCRQDSQKGNHSLNFYRIIFYLKRHVHKWQDAQHTSFYCVSLYCTSQILHFLTNGRFLATLCQANLSVPFVQQHALPSCLWVTFW

SEQ ID NO: 91 - protein, 44 aa, Homo sapiens
RVDQDGHVKLNLALTTETNCNFELLHFPRDHSNCSLSFYALSNT

SEQ ID NO: 92 - protein, 44 aa, Homo sapiens; identical to SEQ ID NO: 91

SEQ ID NO: 93 - protein, 59 aa, Homo sapiens
LEFSPIFYCLRLSSFLWLAYRLSPQPGYLDFLEFSPIFYFLSLSCFLWLAYRLSPQPGY

SEQ ID NO: 94 - protein, 38 aa, Homo sapiens
FNFPPFNLVCFTPHCLLRIDVCTQLFLWTQPTLSLHIL

SEQ ID NO: 95 - protein, 46 aa, Homo sapiens
ASRRCNIVAMCPESVPSGGFLVSLTSRMKPWTLTVSVAVLKDGVSG

SEQ ID NO: 96 - protein, 43 aa, Homo sapiens
GAILTNETWEKLAGELVGYFPFALKGAKERYIPFFFPFSSLDV

SEQ ID NO: 97 - protein, 164 aa, Homo sapiens
KRECHQRRPKEQILTLQEKLWARQKEKDQLFLQLKKVSMRKKNGGERSRATPSDIRCEPAEPDYSRGDSLPPRHAGSAGGHDRPGIVIAADPAKQMFRPHVLTTRKSVGSAAAFAGTPEQAAWAVPLGLLSPYLNMGPHSPMALVGSSEQFSAPWGAFMSQPQP

SEQ ID NO: 98 - protein, 104 aa, Homo sapiens
GSAGGHDRPGIVIAADPAKQMFRPHVLTTRKSVGSAAAFAGTPEQAAWAVPLGLLSPYLNMGPHSPMALVGSSEQFSAPWGAFMSQPQPYVLLGHFQHTQTGFL

SEQ ID NO: 99 - protein, 62 aa, Homo sapiens [sequence omitted]

SEQ ID NO: 100 - protein, 94 aa, Homo sapiens
QEEDIIQESRFYFRGYGLGHCLQARDGGPMEGSGIYSPQPPAPLLREGETTRKLYVDAKRIDTISRAVFPFTFLIFNIFYWVVYKVLRSEDIHQ

SEQ ID NO: 101 - protein, 43 aa, Homo sapiens
ENRCHTVCNSKSDLDVQSSGSFPKAFHVWLPSCSGNTSQVDGG

SEQ ID NO: 102 - protein, 71 aa, Homo sapiens [sequence omitted]

SEQ ID NO: 103 - DNA, 1779 bp, Homo sapiens
tggtaccggtccggaattcccgggatcacgccctgccttggggcccctctcatatagggagcacagggttgctctccttcatctcacacattcgatgtccactacaggaaggggcgttactttcaccatcaattgctcagggtttggccagcacggggcggatcccactgctctgaattcagtgtttaatagaaagcccttccgtccggtcaccaacatcagcgtccccacccaagtcaacatctccttcgcgatgtctgccatcctagatgtgaatgaacagctgcacctcttgtcatcattcctgtggctggaaatggtttgggataacccatttatcagctggaacccagaggaatgtgagggcatcacgaagatgagtatggcagccaagaacctgtggctcccagacattttcatcattgaactcatggatgtggataagaccccaaaaggcctcacagcatatgtaagtaatgaaggtcgcatcaggtataagaaacccatgaaggtggacagtatctgtaacctggacatcttctacttccccttcgaccagcagaactgcacactcaccttcagctcattcctctacacagtggacagcatgttgctggacatggagaaagaagtgtgggaaataacagacgcatcccggaacatccttcagacccatggagaatgggagctcctgggcctcagcaaggccaccgcaaagttgtccaggggaggcaacctgtatgatcagatcgtgttctatgtggccatcaggcgcaggcccagcctctatgtcataaaccttctcgtgcccagtggctttctggttgccatcgatgccctcagcttctacctgccagtgaaaagtgggaatcgtgtcccattcaagataacgctcctgctgggctacaacgtcttcctgctcatgatgagtgacttgctccccaccagtggcacccccctcatcggtgtctacttcgccctgtgcctgtccctgatggtgggcagcctgctggagaccatcttcatcacccacctgctgcacgtggccaccacccagcccccacccctgcctcggtggctccactccctgctgctccactgcaacagcccggggagatgctgtcccactgcgccccagaaggaaaataagggcccgggtctcacccccacccacctgcccggtgtgaaggagccagaggtatcagcagggcagatgccgggccctgcggaggcagagctgacagggggctcagaatggacaagggcccagcgggaacacgaggcccagaagcagcactcagtggagctgtggttgcagttcagccacgcgatggacgccatgctcttccgcctctacctgctcttcatggcctcctctatcatcaccgtcatatgcctctggaacacctaggcaggtgctcacctgccaacttcagtctggagcttctcttgcctccagggactggccaggtctcccccctttcctgagtaccaactatcatatccccaaagatgactgagtctctgctgtattccatgtatcccaatccggtcctgctgatcaattccaatcccagacatttctccctgttcctgcattttgttggcttccttcagtcctaccatatggttctaggtccctcttacgtcatctgcatagcagactatacctcttctgcccgctgacttgcccaataaataattctgcagagaaaaaaaaaaaaaaaaaaaaaaaaaaaaaaaaaaaaaaaaaaaaaaaaaaaaaagggcggccgctct

SEQ ID NO: 104 - DNA, 999 bp, Homo sapiens
ggaattcccgggatggtcaccaacatcagcgtccccacccaagtcaacatctccttcgcgatgtctgccatcctagatgtgaatgaacagctgcacctcttgtcatcattcctgtggctggaaatggtttgggataacccatttatcagctggaacccagaggaatgtgagggcatcacgaagatgagtatggcagccaagaacctgtggctcccagacattttcatcattgaactcatggatgtggataagaccccaaaaggcctcacagcatatgtaagtaatgaaggtcgcatcaggtataagaaacccatgaaggtggacagtatctgtaacctggacatcttctacttccccttcgaccagcagaactgcacactcaccttcagctcattcctctacacagtggacagcatgttgctggacatggagaaagaagtgtgggaaataacagacgcatcccggaacatccttcagacccatggagaatgggagctcctgggcctcagcaaggccaccgcaaagttgtccaggggaggcaacctgtatgatcagatcgtgttctatgtggccatcaggcgcaggcccagcctctatgtcataaaccttctcgtgcccagtggctttctggttgccatcgatgccctcagcttctacctgccagtgaaaagtgggaatcgtgtcccattcaagataacgctcctgctgggctacaacgtcttcctgctcatgatgagtgacttgctccccaccagtggcacccccctcatcggtgtctacttcgccctgtgcctgtccctgatggtgggcagcctgctggagaccatcttcatcacccacctgctgcacgtggccaccacccagcccccacccctgcctcggtggctccactccctgctgctccactgcaacagcccggggagatgctgtcccactgcgccccagaaggaaaataagggcccgggtctcacccccacccacctgcccggtgaggtgtga

SEQ ID NO: 105 - protein, 586 aa, Homo sapiens
GTGPEFPGSRPALGPLSYREHRVALLHLTHSMSTTGRGVTFTINCSGFGQHGADPTALNSVFNRKPFRPVTNISVPTQVNISFAMSAILDVNEQLHLLSSFLWLEMVWDNPFISWNPEECEGITKMSMAAKNLWLPDIFIIELMDVDKTPKGLTAYVSNEGRIRYKKPMKVDSICNLDIFYFPFDQQNCTLTFSSFLYTVDSMLLDMEKEVWEITDASRNILQTHGEWELLGLSKATAKLSRGGNLYDQIVFYVAIRRRPSLYVINLLVPSGFLVAIDALSFYLPVKSGNRVPFKITLLLGYNVFLLMMSDLLPTSGTPLIGVYFALCLSLMVGSLLETIFITHLLHVATTQPPPLPRWLHSLLLHCNSPGRCCPTAPQKENKGPGLTPTHLPGVKEPEVSAGQMPGPAEAELTGGSEWTRAQREHEAQKQHSVELWLQFSHAMDAMLFRLYLLFMASSIITVICLWNTAGAHLPTSVWSFSCLQGLARSPPFPEYQLSYPQRLSLCCIPCIPIRSCSIPIPDISPCSCILLASFSPTIWFVPLTSSAQTIPLLPADLPNKFCREKKKKKKKKKKKKKKKKKRAAA

SEQ ID NO: 106 - protein, 332 aa, Homo sapiens
GIPGMVTNISVPTQVNISFAMSAILDVNEQLHLLSSFLWLEMVWDNPFISWNPEECEGITKMSMAAKNLWLPDIFIIELMDVDKTPKGLTAYVSNEGRIRYKKPMKVDSICNLDIFYFPFDQQNCTLTFSSFLYTVDSMLLDMEKEVWEITDASRNILQTHGEWELLGLSKATAKLSRGGNLYDQIVFYVAIRRRPSLYVINLLVPSGFLVAIDALSFYLPVKSGNRVPFKITLLLGYNVFLLMMSDLLPTSGTPLIGVYFALCLSLMVGSLLETIFITHLLHVATTQPPPLPRWLHSLLLHCNSPGRCCPTAPQKENKGPGLTPTHLPGEV

SEQ ID NO: 107 - DNA, 485 bp, Homo sapiens
ctggaaaggtccatcgcgtggctgaactgcaaccacagctccactgagtgctgcttctgggcctcgtgttcccgctgggcccttgtccattctgagccccctgtcagctctgcctccgcagggcccggcatctgccctgctgatacctctggctccttcacacctacagaaagacagagactcagccatgggctgcaaatgtcacctgtggagggagggagacagggaaggaggcaggagcagagaagtggaggtgggggaagaggaatgtgacttccctcaccgggcaggtgggtggggggtgagacccgggcccttattttccttctggggcgcagtgggacagcatctccccgggctgttgcagtggagcagcagggagtggagccaccgaggcaggggtgggggctgggtggtggccacgtgcagcaggtgggtgatgaagatggtctccagcaggctgcccaccatcagggacaggcaca

SEQ ID NO: 108 - DNA, 584 bp, Homo sapiens
cccagcactttgggaggccaaggtgggtggatcacttcagttcaggagtttgagaccagcctgggcaacatggtgaaacctcatctcttaaaaaaaaaaaaaaaaaaaaaattagccaggcctggtggtgcgcctgtagtcccagctacttgggaggctgaggctgagacaggaggatcatttgagcccaggacatggaagttgcagtgagctgagagcatgccactctactccagcctgggtgacagagcaagatcctgtctcaaaaaaaaaaaaaaaaaaaaaggagagagagaaactgcggcccctgcctcttgcgttatctctcctccagcatggatgtggataaaaccccaaaaggcctcacagcatatgtaagtaatgaaggtcgcatcaggtataaaaaacccatgaagggggacagtatctgtaacctggacatcttctacttccccttcgaccagcaaaactgcacactcaccttcagctcattcctctacacaggtaagttgcagtgaggtctcagggatggggtgaatgagagcaaccaacaaatttaaagaaactatgagtaaatggtgacc

SEQ ID NO: 109 - protein, 38 aa, Homo sapiens
CLSLMVGSLLETIFITHLLHVATTQPPPLPRWLHSLLL

SEQ ID NO: 110 - protein, 60 aa, Homo sapiens
LSSSMDVDKTPKGLTAYVSNEGRIRYKKPMKGDSICNLDIFYFPFDQQNCTLTFSSFLYT

SEQ ID NO: 111 - DNA, 30 bp, Artificial
  Probe/Primer
tgccagtgaaaagtgggaatcgtgtcccat

SEQ ID NO: 112 - DNA, 22 bp, Artificial
  Probe/Primer
cccagcctctatgtcataaacc

SEQ ID NO: 113 - DNA, 20 bp, Artificial
  Probe/Primer
tcatgagcaggaagacgttg

SEQ ID NO: 114 - DNA, 19 bp, Artificial
  Probe/Primer
gccatcaggcgcaggccaa

SEQ ID NO: 115 - DNA, 23 bp, Artificial
  Probe/Primer
caagtcattcatcatgagcagga

SEQ ID NO: 116 - DNA, 20 bp, Artificial
  Probe/Primer
tgcctgtccctgatggtggg

SEQ ID NO: 117 - DNA, 19 bp, Artificial
  Probe/Primer
gagcagcagggagtggagc